(19) United States
(12) Patent Application Publication
(10) Pub. No.: US 2010/0190788 A1
(43) Pub. Date: Jul. 29, 2010

(54) Amide derivatives as kinase inhitors

(76) Inventors: Olivier Defert, Le Havre (FR); Philippe Van Rompaey, Melle (BE); Petra Blom, Destelbergen (BE); Dirk Leysen, Lokeren (BE); Gert De Wilde, Zele (BE); Thomas Brown, Essex (GB)

Correspondence Address: WOLF GREENFIELD & SACKS, P.C., 600 ATLANTIC AVENUE, BOSTON, MA, 02210-2206, US

(21) Appl. No.: 11/988,614

(22) PCT Filed: Jul. 11, 2006
(86) PCT No.: PCT/EP2006/006765

§ 371 Date: Jun. 22, 2009

Related U.S. Application Data
(60) Provisional application No. 60/698,117, filed on Jul. 11, 2005.

(30) Foreign Application Priority Data
Dec. 21, 2005 (EP) PCT/EP2005/013770

(51) Int. Cl. A61K 31/5377; C07D 213/74; A61K 31/4409; A61K 31/444; C07D 401/12; C07D 405/12; A61K 31/443; C07D 409/12; A61K 31/4439; C07D 413/12; A61K 31/496; C07D 471/04; A61K 31/437; C07D 473/34; A61K 31/52; A61P 27/06; A61P 15/10; A61P 29/00; A61P 11/06; A61P 19/10; A61P 13/12; A61P 9/10; A61P 9/12; A61P 7/02; A61P 25/28; A61P 35/00; A61P 35/02; A61P 17/06; A61P 1/04; C12N 9/99

(52) U.S. Cl. 514/235.5; 546/309; 514/352; 514/332; 546/265; 546/283.4; 514/336; 546/280.4; 514/341; 546/275.4; 546/275.1; 546/194; 514/318; 544/131; 544/360; 514/253.01; 546/113; 514/300; 514/303; 546/119; 544/277; 514/263.4; 435/184

(57) ABSTRACT

The present invention relates to new AGC kinase inhibitors, in particular to compounds of Formula (I) or (II) or a stereoisomer, tautomer, racemic, metabolite, pro- or predrug, salt, hydrate, or solvate thereof, wherein Ar¹, Ar², R¹, R³, p and n have the meaning defined in the claims. In particular, the present invention relates to more specifically ROCK inhibitors, compositions, in particular pharmaceuticals, comprising such inhibitors, and to uses of such inhibitors in the treatment and prophylaxis of disease.

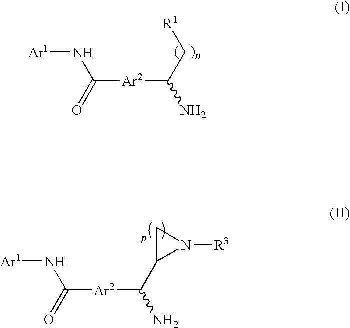

### FIELD OF THE INVENTION

The present invention relates to new kinase inhibitors, more specifically AGC kinases inhibitors, compositions, in particular pharmaceuticals, comprising such inhibitors, and to uses of such inhibitors in the treatment and prophylaxis of disease.

### BACKGROUND OF THE INVENTION

AGC-family protein kinases are named after their family members: protein kinase A (PKA), protein kinase G (PKG), and protein kinase C(PKC).

One AGC-kinase family of interest is Rho-associated coiled-coil forming protein serine/threonine kinase (ROCK), which is believed to be an effector of Ras-related small GTPase Rho. The Rho family consists of at least 10 members of small GTP binding proteins, including RhoA, B, C, D, E, F, G, Rac1, Rac2, Cdc42 and TC10. Two isoforms of ROCK are known: α (ROCKII) and β (ROCKI). ROCKI shows highest expression levels in non-neuronal tissues, such as heart, lung and skeletal muscles; whereas ROCKII is preferentially expressed in brain (hippocampus, cortex and cerebellum).

The Rho/Rho-kinase mediated pathway plays an important role in the signal transduction pathway of many agonists such as angiotensin II, 5-HT, NA, thrombin, endothelin-1, urotensin II, platelet-derived growth factor and ATP/ADP. Activation of ROCK leads to phosphorylation of various proteins: MLCP, MLC, LIMKs, CRMP2 and others. One of the main substrates is the myosin light chain MLC. Activation of MLC, together with the ROCK-induced inactivation of the MLCPhosphatase, leads to stimulation of actin-myosin interactions and subsequent cell contraction and stress fiber formation. ROCK also induces activation of LIMs resulting in an increase of actin filaments. Finally, ROCK activates the ERM protein complex and other proteins involved in cytoskeletal regulation.

In conclusion, ROCKs play an important role in various cellular functions: such as smooth muscle contraction, actin cytoskeleton organization, platelet activation, downregulation of myosin phosphatase cell adhesion, -migration, -proliferation and -survival, thrombin-induced responses of aortic smooth muscle cells, hypertrophy of cardiomyocytes, bronchial smooth muscle contraction, smooth muscle contraction and cytoskeletal reorganization of non-muscle cells, activation of volume-regulated anion channels, neurite retraction, neutrophil chemotaxis, wound healing and cell transformation and gene expression.

More specifically, ROCK has been implicated in various diseases and disorders including hypertension, cerebral vasospasm, coronary vasospasm, bronchial asthma, preterm labor, erectile dysfunction, glaucoma, vascular smooth muscle cell proliferation, myocardial hypertrophy, malignoma, ischemia/reperfusion-induced injury, endothelial dysfunction, Crohn's Disease and colitis, neurite outgrowth, Raynaud's Disease, angina, Alzheimer's disease, benign prostatic hyperplasia and atherosclerosis.

Accordingly, the development of inhibitors of ROCK would be useful as therapeutic agents for the treatment of disorders implicated in the ROCK pathway. Accordingly, there is a great need to develop inhibitors of ROCK that are useful in treating various diseases or conditions associated with ROCK activation, particularly given the inadequate treatments currently available for the majority of these disorders.

### SUMMARY OF THE INVENTION

We have surprisingly found that the compounds described herein act as inhibitors of AGC-kinases and in particular of ROCK.

These compounds and pharmaceutically acceptable compositions thereof are useful for treating or lessening the severity of a variety of disorders, including allergic disorders such as asthma, cardiovascular diseases, vascular diseases, eye diseases, renal diseases, erectile dysfunction, inflammatory diseases, proliferative disorders, neurological disorders and diseases of the central nervous system (CNS), osteoporosis, renal diseases and AIDS.

Viewed from a first aspect, the invention provides a compound of Formula I or II or a stereoisomer, tautomer, racemic, metabolite, pro- or predrug, salt, hydrate, or solvate thereof,

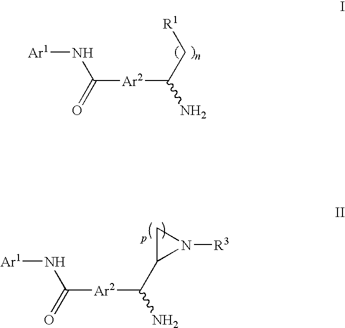

wherein:
Ar¹ is an aromatic 6-membered first ring containing carbon atoms and at least one nitrogen atom, said first ring being optionally fused to a saturated, unsaturated or aromatic 4-, 5-, 6-, or 7-membered second ring containing carbon atoms and optionally at least one nitrogen atom, said first or said second rings being independently substituted with one or more substituents independently selected from the group comprising hydrogen, halogen, alkyl, cycloalkyl, alkenyl, alkynyl, aralkyl, heteroarylalkyl, cycloalkylalkyl, acyl, aryl or heteroaryl wherein said substituents are optionally substituted by one or more further substituents selected from the group comprising halo, hydroxyl, oxo, nitro, amido, carboxy, amino, cyano, haloalkoxy, and haloalkyl;
Ar² is an aromatic 5- or 6-membered third ring containing carbon atoms and optionally one or two heteroatoms, said third ring optionally fused to an aromatic 6-membered fourth ring containing carbon atoms and optionally at least one heteroatom atom, wherein said third ring is optionally substituted with one or more substituents selected from the group comprising halogen, alkenyl, alkyl, alkynyl, acylamino, alkoxy, arylamino, nitro, haloalkoxy, aryl or heteroaryl, wherein said substituents are optionally substituted by one or more further substituents selected from the group comprising halo, hydroxyl, oxo, nitro, amido, carboxy, amino, cyano, haloalkoxy, and haloalkyl;
n is an integer selected from 0 or 1; and
p is an integer selected from 2, 3, 4 or 5; preferably 3 or 4, more preferably 3 and
R¹ is selected from the Formula:

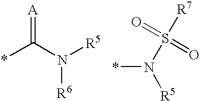

### R³ is of Formula

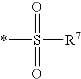

A is an oxygen or sulfur atom;
R⁵ is selected from hydrogen, alkyl, cycloalkyl, cycloalkylalkyl, aryl or aralkyl,
R⁶ and R⁷ are each independently selected from hydrogen, or a group selected from alkoxy, alkyl, alkylamino, alkylaminoalkyl, alkylcarbonyl, alkylcarbonylamino, amino, aralkyl, aryl, carbonylamino, cycloalkyl, formylamino, heteroaryl, heteroarylalkyl, heterocyclyl, or fused to the cycloalkyl, aryl, heterocyclyl or heteroaryl group may be one or more cycloalkyl, aryl, heterocyclyl or heteroaryl, or R⁵ and R⁶ together with the carbon atom to which they are attached form a heterocyclyl ring,
each group or said heterocyclyl ring being optionally substituted by one or more substituent selected from halo, alkenylaminooxy, alkoxy, alkyl, alkylamino, alkylaminosulfonyl, alkylcarbonyl, alkylcarbonylamino, alkyloxyaminoalkenyl, alkyloxycarbonyl, alkylsulfonyl, alkylsulfonylamino, alkylthio, amino, aralkyl, aryl, arylalkenylaminooxy, arylamino, arylaminosulfonyl, arylcarbonyl, arylcarbonylamino, aryloxy, cyano, cycloalkyl, haloalkoxy, haloalkyl, haloaryl, heteroaryl, heteroarylalkenylaminooxy, heteroarylalkyl, heteroarylcarbonylamino, heterocyclyl, hydroxyalkyl, nitro, oxo, sulfonyl, or fused to the cycloalkyl, aryl, heterocyclyl substituent or heteroaryl may be one or more cycloalkyl, aryl, heterocyclyl or heteroaryl,
   each of said substituent being optionally substituted by one or more further substituent selected from halo, alkoxy, alkyl, alkylamino, alkylcarbonyl, alkylheteroaryl, alkylsuphonyl, aralkyl, aryl, arylamino, aryloxy, cyano, haloalkoxy, haloalkyl, heteroaryl, heteroarylalkyl, heteroarylcarbonyl, heterocyclyl, hydroxyl, nitro, oxo, or sulfonyl,
      wherein when n is 1, R⁵ and R⁶ together with the carbon atom to which they are attached form a heterocyclyl or heteroaryl ring optionally substituted with
      one or more substituent selected from halo, alkenylaminooxy, alkoxy, alkyl, alkylamino, alkylaminosulfonyl, alkylcarbonyl, alkylcarbonylamino, alkyloxyaminoalkenyl, alkyloxycarbonyl, alkylsulfonyl, alkylsulfonylamino, alkylthio, amino, aralkyl, aryl, arylalkenylaminooxy, arylamino, arylaminosulfonyl, arylcarbonyl, arylcarbonylamino, aryloxy, cyano, cycloalkyl, haloalkoxy, haloalkyl, haloaryl, heteroaryl, heteroarylcarbonyl, heteroarylalkenylaminooxy, heteroarylalkyl, heteroarylcarbonylamino, heterocyclyl, hydroxyalkyl, nitro, oxo, sulfonyl, or fused to the cycloalkyl, aryl, heterocyclyl or heteroaryl substituent may be one or more cycloalkyl, aryl, heterocyclyl or heteroaryl,
or optionally fused with one or more aryl, heteroaryl, cycloalkyl, or heterocyclyl ring,
   each of said substituent or fused ring being optionally substituted by one or more further substituent selected from halo, alkoxy, alkyl, alkylamino, alkylcarbonyl, alkyloxycarbonyl, alkylheteroaryl, alkylsuphonyl, aralkyl, aryl, arylamino, aryloxy, cyano, haloalkoxy, haloalkyl, heteroaryl, heteroarylalkyl, heteroarylcarbonyl, heterocyclyl, hydroxyl, nitro, oxo, or sulfonyl.

Viewed from a further aspect, the invention provides a pharmaceutical and/or veterinary composition comprising a compound of the invention.

Viewed from a still further aspect, the invention provides a compound of the invention for use in human or veterinary medicine.

Viewed from a still further aspect, the invention provides the use of a compound of the invention in the preparation of a medicament for the prevention and/or treatment of at least one disease and/or disorder selected from the group comprising eye diseases; erectile dysfunction; cardiovascular diseases; vascular diseases; proliferative diseases; inflammatory diseases; neurological diseases and disease of the central nervous system (CNS); bronchial asthma; osteoporosis; renal diseases; and AIDS.

Viewed from a still further aspect, the invention provides the use of a compound of the invention in the preparation of a medicament for the prevention and/or treatment of eyes diseases including macular degeneration, retinopathy and glaucoma, and/or for preventing, treating and/or alleviating complications and/or symptoms associated therewith.

Viewed from a still further aspect, the invention provides the use of a compound of the invention in the preparation of a medicament for the prevention and/or treatment of inflammatory diseases, such as contact dermatitis, psoriasis, rheumatoid arthritis, inflammatory bowel disease, Crohn's disease, ulcerative colitis, and/or for preventing, treating and/or alleviating complications and/or symptoms and/or inflammatory responses associated therewith.

Viewed from a still further aspect, the invention provides the use of a compound of the invention in the preparation of a medicament for the prevention and/or treatment of cardiovascular and vascular diseases including but not limited to acute stroke, congestive heart failure, cardiovascular ischemia, heart disease, cardiac remodeling, angina, coronary vasospasm, cerebral vasospasm, restenosis, hypertension, (pulmonary) hypertension, arteriosclerosis, thrombosis (including deep thrombosis), pulmonary vasoconstriction, and platelet related diseases, and/or for preventing, treating and/or alleviating complications and/or symptoms associated therewith and/or alleviating complications and/or symptoms associated therewith.

Viewed from a still further aspect, the invention provides the use of a compound of the invention in the preparation of a medicament for the prevention, treatment and/or management of neurological and CNS disorders including but not limited to stroke, multiple sclerosis, brain or spinal cord injury, inflammatory and demyelinating diseases such as Alzheimer's disease, MS and neuropathic pain, and/or for preventing, treating and/or alleviating complications and/or symptoms associated therewith.

Viewed from a still further aspect, the invention provides the use of a compound of the invention in the preparation of a medicament for the prevention and/or treatment of proliferative diseases such as including but not limited to cancer of the brain (gliomas), breast, colon, intestine, skin, head and neck, kidney, lung, liver, ovarian, pancreatic, prostate or thyroid; leukemia; sarcoma; lymphoma; melanoma; and/or for preventing, treating and/or alleviating complications and/or symptoms and/or inflammatory responses associated therewith.

Viewed from a still further aspect, the invention provides the use of a compound of the invention in the preparation of a medicament for the prevention and/or treatment of erectile dysfunction, bronchial asthma, osteoporosis, renal diseases and AIDS, and/or for preventing, treating and/or alleviating complications and/or symptoms associated therewith.

Viewed from a still further aspect, the invention provides the use of a compound of the invention, or a composition comprising such a compound, for inhibiting the activity of at least one kinase, in vitro or in vivo.

Viewed from a still further aspect, the invention provides the use of a compound of the invention, or a composition comprising such a compound, for inhibiting the activity of at least one ROCK kinase, for example ROCKII and/or ROCKI isoforms.

### DETAILED DESCRIPTION OF THE INVENTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Unless a context dictates otherwise, asterisks are used herein to indicate the point at which a mono- or bivalent radical depicted is connected to the structure to which it relates and of which the radical forms part.

Undefined (racemic) asymmetric centers that may be present in the compounds of Formula I or II are interchangeably indicated by drawing a wavy bonds or a straight bond in order to visualize the undefined steric character of the bond, for example

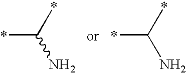

are used for the carbon bearing the amine of compounds of Formula I or II.

In an embodiment, the present invention provides a compound of Formula I or II:

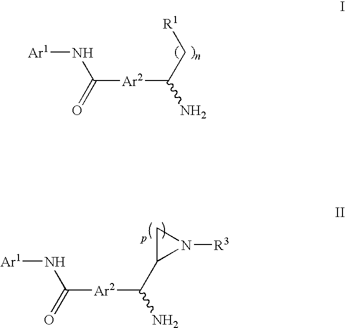

wherein:
Ar¹ is an aromatic 6-membered first ring containing carbon atoms and at least one nitrogen atom, said first ring being optionally fused to a saturated, unsaturated or aromatic 4-, 5-, 6-, or 7-membered second ring containing carbon atoms and optionally at least one nitrogen atom, said first or said second rings being independently substituted with one or more substituents (for example 1, 2, 3 or 4) independently selected from the group comprising hydrogen, halogen, alkyl, cycloalkyl, alkenyl, alkynyl, aralkyl, heteroarylalkyl, cycloalkylalkyl, acyl, aryl or heteroaryl wherein said substituents are optionally substituted by one or more further substituents selected from the group comprising halo, hydroxyl, oxo, nitro, amido, carboxy, amino, cyano, haloalkoxy, and haloalkyl;
Ar² is an aromatic 5- or 6-membered third ring containing carbon atoms and optionally one or two heteroatoms, said third ring optionally fused to an aromatic 6-membered fourth ring containing carbon atoms and optionally one or two heteroatoms, wherein said third ring is optionally substituted with one or more substituents (for example 1, 2, 3 or 4) selected from the group comprising halogen, alkenyl, alkyl, alkynyl, acylamino, alkoxy, arylamino, nitro, haloalkoxy, aryl or heteroaryl, optionally substituted by one or more substituents;
n is an integer selected from 0 or 1 and
p is an integer selected from 2, 3, 4 or 5, preferably 3 or 4, more preferably 3,
   R¹ is selected from the Formula:

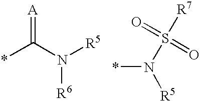

R³ is of Formula

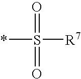

wherein
A is an oxygen or sulfur atom,
R⁵, R⁶ and R⁷ are each independently selected from the group comprising:
(A) hydrogen;
(B) alkyl, alkenyl or alkynyl, optionally substituted with:
(i) a homocyclic, heterocyclic, aryl or heteroaryl ring, to which may be fused one or more homo or heterocyclic, aryl or heteroaryl rings, and which said ring or said one or more optional rings may be optionally substituted with one or more substituents independently selected from a first group comprising alkyl, hydroxyalkyl, haloalkyl, alkenyl, alkynyl, and homocyclic, heterocyclic, aryl or heteroaryl rings, wherein any substituents from this first group may be attached though an oxygen, sulfur or nitrogen atom or though one carbon atom; or independently selected from a second group comprising halo, oxo, nitro, amido, carboxy, hydroxyl, amino, cyano and haloalkoxy; or
(ii) a substituent selected from the second group defined in part (i); or
(iii) a substituent selected from the first group as defined in part (i) wherein said substituent is attached though an oxygen, sulfur or nitrogen atom or though one carbon atom, and wherein said a homocyclic, heterocyclic, aryl or heteroaryl rings are as defined in part (i);
(C) homocyclic and heterocyclic rings optionally substituted with:
(iv) a homocyclic, heterocyclic, aryl or heteroaryl ring as defined in part (i); or
(v) a substituent selected from the second group as defined in part (i); or
(vi) a substituent selected from the first group as defined in part (i) wherein said alkyl, hydroxyalkyl, haloalkyl, alkenyl, alkynyl, and homocyclic, heterocyclic, aryl or heteroaryl rings may, if present, be attached though an oxygen, sulfur or nitrogen atom or though one carbon atom, and wherein said homocyclic, heterocyclic, aryl or heteroaryl rings are as defined in part (i); and
(vii) where the homocyclic or heterocyclic rings comprise 4 or more ring atoms, fused to the homocyclic and heterocyclic rings may be one or more homo or heterocyclic, aryl or heteroaryl rings, and said rings, if present, may be optionally substituted with one or more substituents independently selected from the first or a second groups as defined in part (i) wherein said substituents in said second group may, if present, be attached though an oxygen, sulfur or nitrogen atom or though one carbon atom; and
(D) an aryl or heteroaryl ring optionally substituted with:
(viii) a homocyclic, heterocyclic, aryl or heteroaryl ring as defined in part (i); or
(ix) a substituent selected from the second group as defined in part (i); or
(x) a substituent selected from the first group as defined in part (i) wherein said alkyl, hydroxyalkyl, haloalkyl, alkenyl, alkynyl, and homocyclic, heterocyclic, aryl or heteroaryl rings may, if present, be attached though an oxygen, sulfur or nitrogen atom or though one carbon atom, and wherein said homocyclic, heterocyclic, aryl or heteroaryl rings are as defined in part (i); and
(xi) fused to the aryl or heteroaryl ring may be one or more homo or heterocyclic, aryl or heteroaryl rings, and said rings, if present, may be optionally substituted with one or more substituents independently selected from the first or a second groups as defined in part (i) wherein said substituents in said second group may, if present, be attached though an oxygen, sulfur or nitrogen atom or though one carbon atom;
or R⁵ and R⁶ may together with the common nitrogen atom to which they are attached form a heterocyclic ring, or when n is 1, R⁵ and R⁶ may together with the common nitrogen atom to which they are attached form a heterocyclic ring, said ring being optionally substituted with:
(xii) an alkyl, alkenyl or alkynyl as defined in part (B); a homocyclic or heterocyclic ring as defined in part (C); or an aryl or heteroaryl ring as defined in part (D); and
(xiii) where the said optionally substituted heterocyclic ring formed from R⁵ and R⁶ and the common nitrogen atom to which they are attached comprises 4 or more ring atoms, fused to it may be one or more homo or heterocyclic, aryl or heteroaryl rings, and said rings, if present, may be optionally substituted with one or more substituents independently selected from an alkyl, alkenyl or alkynyl as defined in part (B); a homocyclic or heterocyclic ring as defined in part (C); or an aryl or heteroaryl ring as defined in part (D),
or a stereoisomer, tautomer, racemic, metabolite, pro- or predrug, salt, hydrate, or solvate thereof.

When describing the compounds of the invention, the terms used are to be construed in accordance with the following definitions, unless a context dictates otherwise:

The term “alkyl” by itself or as part of another substituent refers to a hydrocarbyl radical of Formula CₙH₂ₙ₊₁ wherein n is a number greater than or equal to 1. Generally, alkyl groups of this invention comprise from 1 to 20 carbon atoms, more preferably from 1 to 10 carbon atoms, still more preferably 1 to 8 carbon atoms, in particular 1 to 6 carbon atoms, preferably 1 to 4 carbon atoms. Alkyl groups may be linear or branched and may be substituted as indicated herein. When a subscript is used herein following a carbon atom, the subscript refers to the number of carbon atoms that the named group may contain. Thus, for example, C₁₋₄alkyl means an alkyl of one to four carbon atoms. Examples of alkyl groups are methyl, ethyl, n-propyl, i-propyl, butyl and its isomers (e.g. n-butyl, i-butyl and t-butyl); pentyl and its isomers, hexyl and its isomers, heptyl and its isomers, octyl and its isomers, nonyl and its isomers; decyl and its isomers. C₁-C₆ alkyl includes all linear, branched or cyclic alkyl groups with between 1 and 6 carbon atoms, and thus includes methyl, ethyl, n-propyl, i-propyl, butyl and its isomers (e.g. n-butyl, i-butyl and t-butyl); pentyl and its isomers, hexyl and its isomers, cyclopentyl, 2-, 3- or 4-methylcyclopentyl, cyclopentylmethylene, and cyclohexyl.

The term “optionally substituted alkyl” refers to an alkyl group optionally substituted with one or more substituents (for example 1 to 4 substituents, or example 1, 2, 3 or 4 substituents or 1 to 2 substituents) at any available point of attachment. Non-limiting examples of such substituents include halogen, hydroxyl, carbonyl, nitro, amino, oximes, imines, azido, hydrazino, cyano, alkyl, aryl, heteroaryl, cycloalkyl, acyl, alkylamino, alkoxy, thio, alkylthio, carboxylic acid, acylamino, alkyl esters, carbamates, thioamides, urea, sulphonamides and the like.

When the term “alkyl” is used as a suffix following another term, as in “hydroxyalkyl,” this is intended to refer to an alkyl group, as defined above, being substituted with one or two (preferably one) substituent(s) selected from the other, specifically-named group, also as defined herein. The term “hydroxyalkyl” refers to a —R^{a}—OH group wherein R^{a} is alkylene as defined herein. For example, “hydroxyalkyl” includes 2-hydroxyethyl, 1-(hydroxymethyl)-2-methylpropyl, 3,4-dihydroxybutyl, and so forth. “Alkoxyalkyl” refers to an alkyl group substituted with one to two of OR′, wherein R′ is alkoxy as defined below. For example, “aralkyl” or “(aryl)alkyl” refers to a substituted alkyl group as defined above wherein at least one of the alkyl substituents is an aryl as defined below, such as benzyl. For example, “heteroarylalkyl” refers to a substituted alkyl group as defined above wherein at least one of the alkyl substituents is a heteroaryl as defined below, such as pyridinyl.

The term “cycloalkyl group” as used herein is a cyclic alkyl group, that is to say, a monovalent, hydrocarbyl group having 1, 2 or 3 cyclic structure. Cycloalkyl includes all saturated or partially saturated (containing 1 or 2 double bonds) hydrocarbon groups containing 1 to 3 rings, including monocyclic, bicyclic or polycyclic alkyl groups. Cycloalkyl groups may comprise 3 or more carbon atoms in the ring and generally, according to this invention comprise from 3 to 10, more preferably from 3 to 8 carbon atoms still more preferably from 3 to 6 carbon atoms. The further rings of multi-ring cycloalkyls may be either fused, bridged and/or joined through one or more spiro atoms. Cycloalkyl groups may also be considered to be a subset of homocyclic rings discussed hereinafter. Examples of cycloalkyl groups, are cyclopropyl, cyclobutyl, cyclopentyl, cyclohexyl, cycloheptyl, cyclooctyl, cyclononyl and cyclodecyl with cyclopropyl being particularly preferred. An “optionally substituted cycloalkyl” refers to a cycloalkyl having optionally one or more substituents (for example 1 to 3 substituents, or 1 to 2 substituents), selected from those defined above for substituted alkyl. When the suffix “ene” is used in conjunction with a cyclic group, this is intended to mean the cyclic group as defined herein having two single bonds as points of attachment to other groups.

Where alkyl groups as defined are divalent, i.e., with two single bonds for attachment to two other groups, they are termed “alkylene” groups. Non-limiting examples of alkylene groups includes methylene, ethylene, methylmethylene, trimethylene, propylene, tetramethylene, ethylethylene, 1,2-dimethylethylene, pentamethylene and hexamethylene. Similarly, where alkenyl groups as defined above and alkynyl groups as defined above, respectively, are divalent radicals having single bonds for attachment to two other groups, they are termed “alkenylene” and “alkynylene” respectively.

Generally, alkylene groups of this invention preferably comprise the same number of carbon atoms as their alkyl counterparts. “Cycloalkylene” herein refers to a saturated homocyclic hydrocarbyl biradical of Formula CₙH₂ₙ₋₂. Cycloalkylene groups of this invention preferably comprise the same number of carbon atoms as their cycloalkyl radical counterparts. Where an alkylene or cycloalkylene biradical is present, connectivity to the molecular structure of which it forms part may be through a common carbon atom or different carbon atom, preferably a common carbon atom. To illustrate this applying the asterisk nomenclature of this invention, a C₃ alkylene group may be for example *—CH₂CH₂CH₂—*, *—CH(—CH₂CH₃)—* or *—CH₂CH(—CH₃)—*. Likewise a C₃ cycloalkylene group may be

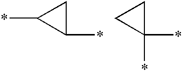

Where a cycloalkylene group is present, this is preferably a C₃-C₆ cycloalkylene group, more preferably a C₃ cycloalkylene (i.e. cyclopropylene group) wherein its connectivity to the structure of which it forms part is through a common carbon atom. Cycloalkylene and alkylene biradicals in compounds of the invention may be, but preferably are not, substituted.

The term “alkenyl” as used herein refers to an unsaturated hydrocarbyl group, which may be linear, branched or cyclic, comprising one or more carbon-carbon double bonds. Alkenyl groups thus comprise two or more carbon atoms, preferably between 2 and 20 carbon atoms, more preferably between 2 and 10 carbon atoms, still more preferably between 2 and 8 carbon atoms, for example, between 2 and 6 carbon atoms. Similarly to cycloalkyl groups, cycloalkenyl groups may be considered to be a subset of homocyclic rings discussed hereinafter. Examples of alkenyl groups are ethenyl, 2-propenyl, 2-butenyl, 3-butenyl, 2-pentenyl and its isomers, 2-hexenyl and its isomers, 2-heptenyl and its isomers, 2-octenyl and its isomers, 2,4-pentadienyl and the like. An optionally substituted alkenyl refers to an alkenyl having optionally one or more substituents (for example 1, 2 or 3 substituents, or 1 to 2 substituents), selected from those defined above for substituted alkyl. Similarly to cycloalkyl groups, cycloalkenyl groups may be considered to be a subset of homocyclic rings discussed hereinafter.

The term “alkynyl” as used herein, similarly to alkenyl, refers to a class of monovalent unsaturated hydrocarbyl groups, wherein the unsaturation arises from the presence of one or more carbon-carbon triple bonds. Alkynyl groups typically, and preferably, have the same number of carbon atoms as described above in relation to alkenyl groups. Examples alkynyl groups are ethynyl, 2-propynyl, 2-butynyl, 3-butynyl, 2-pentynyl and its isomers, 2-hexynyl and its isomers, 2-heptynyl and its isomers, 2-octynyl and its isomers and the like. An optionally substituted alkynyl refers to an alkynyl having optionally one or more substituents (for example 1 to 4 substituents, or 1 to 2 substituents), selected from those defined above for substituted alkyl. Similarly to cycloalkyl groups, cycloalkynyl groups may be considered to be a subset of homocyclic rings discussed hereinafter.

The term “homocyclic ring” as used herein is a ring wherein the ring atoms comprise only carbon atoms. Examples of homocyclic rings thus include cycloalkyl, cycloalkenyl and cycloalkynyl, with cycloalkyl and cycloalkenyl being preferred. Where a ring carbon atom is replaced with a heteroatom, preferably nitrogen, oxygen of sulfur, the heteroatom-containing ring resultant from such a replacement is referred to herein as a heterocyclic ring. More than one carbon atom in a ring may be replaced so forming heterocyclic ring having a plurality of heteroatoms.

The terms “heterocyclyl” or “heterocyclo” as used herein by itself or as part of another group refer to non-aromatic, fully saturated or partially unsaturated cyclic groups (for example, 3 to 13 member monocyclic, 7 to 17 member bicyclic, or 10 to 20 member tricyclic ring systems, or containing a total of 3 to 10 ring atoms) which have at least one heteroatom in at least one carbon atom-containing ring. Each ring of the heterocyclic group containing a heteroatom may have 1, 2, 3 or 4 heteroatoms selected from nitrogen atoms, oxygen atoms and/or sulfur atoms, where the nitrogen and sulfur heteroatoms may optionally be oxidized and the nitrogen heteroatoms may optionally be quaternized. The heterocyclic group may be attached at any heteroatom or carbon atom of the ring or ring system, where valence allows. The rings of multi-ring heterocycles may be fused, bridged and/or joined through one or more spiro atoms. An optionally substituted heterocyclic refers to a heterocyclic having optionally one or more substituents (for example 1 to 4 substituents, or for example 1, 2, 3 or 4), selected from those defined above for substituted aryl.

Exemplary heterocyclic groups include piperidinyl, azetidinyl, imidazolinyl, imidazolidinyl, isoxazolinyl, oxazolidinyl, isoxazolidinyl, thiazolidinyl, isothiazolidinyl, piperidyl, succinimidyl, 3H-indolyl, indolinyl, isoindolinyl, chromenyl, isochromanyl, xanthenyl, 2H-pyrrolyl, 1-pyrrolinyl, 2-pyrrolinyl, 3-pyrrolinyl, pyrrolidinyl, 4H-quinolizinyl, 4aH-carbazolyl, 2-oxopiperazinyl, piperazinyl, homopiperazinyl, 2-pyrazolinyl, 3-pyrazolinyl, pyranyl, dihydro-2H-pyranyl, 4H-pyranyl, 3,4-dihydro-2H-pyranyl, triazinyl, cinnolinyl, phthalazinyl, oxetanyl, thietanyl, 3-dioxolanyl, 1,4-dioxanyl, 2,5-dioximidazolidinyl, 2,2,4-piperidonyl, 2-oxopiperidinyl, 2-oxopyrrolodinyl, 2-oxoazepinyl, indolinyl, tetrahydropyranyl, tetrahydrofuranyl, tetrehydrothienyl, tetrahydroquinolinyl, tetrahydroisoquinolinyl, thiomorpholinyl, thiomorpholinyl sulfoxide, thiomorpholinyl sulfone, 1,3-dioxolanyl, 1,4-oxathianyl, 1,4-dithianyl, 1,3,5-trioxanyl, 6H-1,2,5-thiadiazinyl, 2H-1,5,2-dithiazinyl, 2H-oxocinyl, 1H-pyrrolizinyl, tetrahydro-1,1-dioxothienyl, N-formylpiperazinyl, and morpholinyl.

The term “aryl” as used herein refers to a polyunsaturated, aromatic hydrocarbyl group having a single ring (i.e. phenyl) or multiple aromatic rings fused together (e.g. naphthalene or anthracene) or linked covalently, typically containing 5 to 8 atoms; wherein at least one ring is aromatic. The aromatic ring may optionally include one to three additional rings (either cycloalkyl, heterocyclyl or heteroaryl) fused thereto. Aryl is also intended to include the partially hydrogenated derivatives of the carbocyclic systems enumerated herein. Non-limiting examples of aryl comprise phenyl, biphenylyl, biphenylenyl, 5- or 6-tetralinyl, 1-, 2-, 3-, 4-, 5-, 6-, 7- or 8-azulenyl, 1- or 2-naphthyl, 1-, 2- or 3-indenyl, 1-, 2- or 9-anthryl, 1- 2-, 3-, 4- or 5-acenaphtylenyl, 3-, 4- or 5-acenaphtenyl, 1-, 2-, 3-, 4- or 10-phenanthryl, 1- or 2-pentalenyl, 1, 2-, 3- or 4-fluorenyl, 4- or 5-indanyl, 5-, 6-, 7- or 8-tetrahydronaphthyl, 1,2,3,4-tetrahydronaphthyl, 1,4-dihydronaphthyl, dibenzo[a,d]cylcoheptenyl, 1-, 2-, 3-, 4- or 5-pyrenyl.

The aryl ring can optionally be substituted by one or more aromatic substituents. An “optionally substituted aryl” refers to an aryl having optionally one or more substituents (for example 1 to 5 substituents, or 1 to 2 substituents) at any available point of attachment. Non-limiting examples of such substituents are selected from halogen, hydroxyl, oxo, nitro, amino, hydrazine, aminocarbonyl, azido, cyano, alkyl, cycloalkyl, alkenyl, alkynyl, cycloalkylalkyl, alkylamino, alkoxy, —SO₂—NH₂, aryl, heteroaryl, aralkyl, haloalkyl, haloalkoxy, alkyloxycarbonyl, alkylaminocarbonyl, heteroarylalkyl, alkylsulfonamide, heterocyclyl, alkylcarbonylaminoalkyl, aryloxy, alkylcarbonyl, acyl, arylcarbonyl, aminocarbonyl, alkylsulfoxide, —SO₂R¹⁵, alkylthio, carboxy, and the like, wherein R¹⁵ is alkyl or cycloalkyl.

The term “arylene” as used herein is intended to include divalent carbocyclic aromatic ring systems such as phenylene, biphenylylene, naphthylene, anthracenylene, phenanthrenylene, fluorenylene, indenylene, pentalenylene, azulenylene and the like. Arylene is also intended to include the partially hydrogenated derivatives of the carbocyclic systems enumerated above. Non-limiting examples of such partially hydrogenated derivatives are 1,2,3,4-tetrahydronaphthylene, 1,4-dihydronaphthylene and the like.

Where a carbon atom in an aryl group is replaced with a heteroatom, the resultant ring is referred to herein as a heteroaryl ring.

The term “heteroaryl” as used herein by itself or as part of another group refers but is not limited to 5 to 12 carbon-atom aromatic rings or ring systems containing 1 to 3 rings which are fused together or linked covalently, typically containing 5 to 8 atoms; at least one of which is aromatic in which one or more carbon atoms in one or more of these rings can be replaced by oxygen, nitrogen or sulfur atoms where the nitrogen and sulfur heteroatoms may optionally be oxidized and the nitrogen heteroatoms may optionally be quaternized. Such rings may be fused to an aryl, cycloalkyl, heteroaryl or heterocyclyl ring. Non-limiting examples of heteroaryl can be 2- or 3-furyl, 2- or 3-thienyl, 1-, 2- or 3-pyrrolyl, 1-, 2-, 4- or 5-imidazolyl, 1-, 3-, 4- or 5-pyrazolyl, 3-, 4- or 5-isoxazolyl, 2-, 4- or 5-oxazolyl, 3-, 4- or 5-isothiazolyl, 2-, 4- or 5-thiazolyl, 1,2,3-triazol-1-, -2-, -4- or -5-yl, 1,2,4-triazol-1-, -3-, -4- or -5-yl, 1,2,3-oxadiazol-4- or -5-yl, 1,2,4-oxadiazol-3- or -5-yl, 1,2,5-oxadiazolyl, 1,3,4-oxadiazolyl, 1,2,3-thiadiazol-4- or -5-yl, 1,2,4-thiadiazol-3- or -5-yl, 1,2,5-thiadiazol-3- or -4-yl, 1,3,4-thiadiazolyl, 1- or 5-tetrazolyl, 2-, 3- or 4-pyridyl, 3- or 4-pyridazinyl, 2-, 4-, 5- or 6-pyrimidinyl, 2-, 3-, 4-, 5- 6-2H-thiopyranyl, 2-, 3- or 4-4H-thiopyranyl, 2-, 3-, 4-, 5-, 6- or 7-benzofuryl, 1-, 3-, 4- or 5-isobenzofuryl, 2-, 3-, 4-, 5-, 6- or 7-benzothienyl, 1-, 3-, 4- or 5-isobenzothienyl, 1-, 2-, 3-, 4-, 5-, 6- or 7-indolyl, 2- or 3-pyrazinyl, 1,4-oxazin-2- or -3-yl, 1,4-dioxin-2- or -3-yl, 1,4-thiazin-2- or -3-yl, 1,2,3-triazinyl, 1,2,4-triazinyl, 1,3,5-triazin-2-, -4- or -6-yl, thieno[2,3-b]furan-2-, -3-, -4-, or -5-yl, 1-, 2-, 4- or 5-benzimidazolyl, 1-, 3-, 4-, 5-, 6- or 7-benzopyrazolyl, 3-, 4-, 5-, 6- or 7-benzisoxazolyl, 2-, 4-, 5-, 6- or 7-benzoxazolyl, 3-, 4-, 5-, 6- or 7-benzisothiazolyl, 2-, 4-, 5-, 6- or 7-benzothiazolyl, 1-, 2-thianthrenyl, 3-, 4- or 5-isobenzofuranyl, 1-, 2-, 3-, 4- or 9-xanthenyl, 1-, 2-, 3- or 4-phenoxathiinyl, 2-, 3-pyrazinyl, 1-, 2-, 3-, 4-, 5-, 6-, 7- or 8-indolizinyl, 2-, 3-, 4- or 5-isoindolyl, 1-, 2-, 3-, 4-, 5-, 6- or 7-indazolyl, 2-, 6-, 7- or 8-purinyl, 4-, 5- or 6-phthalazinyl, 2-, 3- or 4-naphthyridinyl, 2-, 5- or 6-quinoxalinyl, 2-, 4-, 5-, 6-, 7- or 8-quinazolinyl, 1-, 2-, 3- or 4-quinolizinyl, 2-, 3-, 4-, 5-, 6-, 7-, or 8-quinolinyl(quinolyl), 2-, 4-, 5-, 6-, 7- or 8-quinazolyl, 1-, 3-, 4-, 5-, 6-, 7- or 8-isoquinolinyl(isoquinolyl), 3-, 4-, 5-, 6-, 7- or 8-cinnolinyl, 2-, 4-, 6- or 7-pteridinyl, 1-, 2-, 3-, 4- or 9-carbazolyl, 1-, 2-, 3-, 4-, 5-, 6-, 7-, 8- or 9-carbolinyl, 1-, 2-, 3-, 4-, 5-, 6-, 7-, 8-, 9- or 10-phenanthridinyl, 1-, 2-, 3- or 4-acridinyl, 1-, 2-, 3-, 4-, 5-, 6-, 7-, 8- or 9-perimidinyl, 2-, 3-, 4-, 5-, 6-, 7-, 8-, 9- or 10-(1,7)phenanthrolinyl, 1- or 2-phenazinyl, 1-, 2-, 3-, 4-, or 10-phenothiazinyl, 3- or 4-furazanyl, 1-, 2-, 3-, 4-, or 10-phenoxazinyl, or additionally substituted derivatives thereof.

An “optionally substituted heteroaryl” refers to a heteroaryl having optionally one or more substituents (for example 1 to 4 substituents, or 1 to 2 substituents), selected from those defined above for substituted aryl.

The term “oxo” as used herein refers to the group ═O.

The term “alkoxy” as used herein refers to a radical having the Formula —OR wherein R is alkyl. Preferably, alkoxy is C₁-C₁₀ alkoxy or C₁-C₆ alkoxy. Where the oxygen atom in an alkoxy group is substituted with sulfur, the resultant radical is referred to as thioalkoxy. Haloalkoxy is an alkoxy group wherein one or more hydrogen atoms in the alkyl group are substituted with halo.

The term “aryloxy” as used herein denotes a group —O-aryl, wherein aryl is as defined above.

The term “aroyl” as used herein denotes a group —C(O)-aryl, wherein aryl is as defined above.

The term “cycloalkylalkyl” by itself or as part of another substituent refers to a group having one of the aforementioned cycloalkyl groups attached to one of the aforementioned alkyl chains. Examples of such cycloalkylalkyl radicals include cyclopropylmethyl, cyclobutylmethyl, cyclopentylmethyl, cyclohexylmethyl, 1-cyclopentylethyl, 1-cyclohexylethyl, 2-cyclopentylethyl, 2-cyclohexylethyl, cyclobutylpropyl, cyclopentylpropyl, 3-cyclopentylbutyl, cyclohexylbutyl and the like.

The term “heterocyclyl-alkyl” by itself or as part of another substituents refers to a group having one of the aforementioned heterocyclyl group attached to one of the aforementioned alkyl group, i.e., to a group —R^{b}-R^{c} wherein R^{b} is alkylene or alkylene substituted by alkyl group and R^{c} is a heterocyclyl group.

The term “acyl” by itself or as part of another substituent refers to an alkanoyl group having 2 to 6 carbon atoms or a phenylalkanoyl group whose alkanoyl moiety has 1 to 4 carbon atoms, i.e. a carbonyl group linked to a radical such as, but not limited to, alkyl, aryl, more particularly, the group —COR¹⁰, wherein R¹⁰ can be selected from alkyl, aryl, substituted alkyl, or substituted aryl, as defined herein. The term acyl therefore encompasses the group alkylcarbonyl (—COR¹⁰), wherein R¹⁰ is alkyl. Preferably, acyl is C₂-C₁₁ acyl or C₂-C₇ acyl. Where the oxygen atom is an acyl group is substituted with sulfur, the resultant radical is referred to as thioacyl. Said acyl can be exemplified by acetyl, propionyl, butyryl, valeryl and pivaloyl, benzoyl, phenylacetyl, phenylpropionyl and phenylbutylyl.

The term “amino” refers to the group —NH₂.

The term “alkylamino” by itself or as part of another substituent refers to a group consisting of an amino groups attached to one or two independently selected and optionally substituted alkyl groups, cycloalkyl groups, aralkyl or cycloalkylalkyl groups i.e., alkyl amino refers to —N(R⁸)(R⁹) wherein R⁸ and R⁹ are each independently selected from hydrogen, cycloalkyl, aralkyl, cycloalkylalky or alkyl. Non-limiting examples of alkylamino groups include methylamino (NHCH₃), ethylamino (NHCH₂CH₃), n-propylamino, isopropylamino, n-butylamino, isobutylamino, sec-butylamino, tert-butylamino, n-hexylamino, and the like.

The term “aminoalkyl” refers to the group —R^{b}—NR^{d}R^{e} wherein R^{b} is alkylene or substituted alkylene, R^{d} is hydrogen or alkyl or substituted alkyl as defined herein, and R^{e} is hydrogen or alkyl as defined herein.

The term “aminocarbonyl” refers to the group —(C═O)—NH₂.

The term “alkylaminocarbonyl” refers to a group —(C═O)—NR^{d}R^{e} wherein R^{d} is hydrogen or alkyl or substituted alkyl as defined herein, and R^{e} is alkyl or substituted alkyl as defined herein.

The term “alkylaminocarbonylamino” refers to a group —NH(C═O)—NR^{d}R^{e} or —NR′(C═O)—NR^{d}R^{e} wherein R^{d} is hydrogen or alkyl or substituted alkyl as defined herein, and R^{e} is alkyl or substituted alkyl as defined herein, wherein R′ is alkyl or substituted alkyl.

The term “carboxy” or “carboxyl” refers to the group —CO₂H. Thus, a carboxyalkyl is an alkyl group as defined above having at least one substituent that is —CO₂H.

The term “alkoxycarbonyl” refers to a carboxy group linked to an alkyl radical i.e. to form —C(═O)OR¹⁰, wherein R¹⁰ is as defined above for acyl.

The term “alkylcarbonyloxy” refers to a —O—C(═O)R¹¹ wherein R¹¹ is as defined above for acyl.

The term “alkylcarbonylamino” refers to an group of Formula —NH(C═O)R or —NR′(C═O)R, wherein R and R′ are each independently alkyl or substituted alkyl.

The term “alkylcarbonylaminoalkyl” refers to a group —R^{b}—NR^{d}—C(═O)—R^{e} wherein R^{b} is alkylene or substituted alkylene, R^{d} is hydrogen or alkyl as defined herein, and R^{e} is alkyl as defined herein.

The term “alkoxy” by itself or as part of another substituent refers to a group consisting of an oxygen atom attached to one optionally substituted straight or branched alkyl group, cycloalkyl group, aralkyl or cycloalkylalkyl group. Non-limiting examples of suitable alkoxy group include methoxy, ethoxy, n-propoxy, isopropoxy, n-butoxy, iso-butoxy, sec-butoxy, tert-butoxy, hexanoxy and the like.

The term “alkylthio” by itself or as part of another substituent refers to a group consisting of a sulfur atom attached to one optionally substituted alkyl group, cycloalkyl group, aralkyl or cycloalkylalkyl group. Non-limiting examples of alkylthio groups include methylthio (SCH₃), ethylthio (SCH₂CH₃), n-propylthio, isopropylthio, n-butylthio, isobutylthio, sec-butylthio, tert-butylthio, n-hexylthio, and the like.

The term “acylamino” by itself or as part of another substituent refers to a group consisting of an amino group attached to one or two independently selected acyl groups as described before. In case the two acyl groups of a dicarboxylic acid are attached to the amino group these represent imides such as phtalimides, maleimides and the like, and are encompassed in the meaning of the term acylamino.

The term “halo” or “halogen” as a group or part of a group is generic for fluoro, chloro, bromo or iodo.

The term “haloalkyl” alone or in combination, refers to an alkyl radical having the meaning as defined above wherein one or more hydrogens are replaced with a halogen as defined above. Non-limiting examples of such haloalkyl radicals include chloromethyl, 1-bromoethyl, fluoromethyl, difluoromethyl, trifluoromethyl, 1,1,1-trifluoroethyl and the like.

The term “haloalkoxy” alone or in combination refers to a group of Formula —O-alkyl wherein the alkyl group is substituted by 1, 2 or 3 halogen atoms. For example, “haloalkoxy” includes —OCF₃ and —OCHF₂.

The term “sulfonamide” alone or in combination refers to a group of Formula SO₂—NRR wherein each R independently is hydrogen or alkyl as defined herein.

The term “alkylsulfonylamino” alone or in combination refers to a group of Formula —NR^{d}—SO₂—R wherein R^{d} is hydrogen or alkyl as defined herein, and R independently is alkyl as defined herein.

Whenever the term “substituted” is used in the present invention, it is meant to indicate that one or more hydrogens on the atom indicated in the expression using “substituted” is replaced with a selection from the indicated group, provided that the indicated atom's normal valency is not exceeded, and that the substitution results in a chemically stable compound, i.e. a compound that is sufficiently robust to survive isolation to a useful degree of purity from a reaction mixture, and formulation into a therapeutic agent.

Where groups may be optionally substituted, such groups may be substituted with once or more, and preferably once or twice. Substituents may be selected from, for example, the group comprising halo, hydroxyl, oxo, nitro, amido, carboxy, amino, cyano haloalkoxy, and haloalkyl.

As used herein the terms such as “alkyl, aryl, or cycloalkyl, each being optionally substituted with” or “alkyl, aryl, or cycloalkyl, optionally substituted with” refers to optionally substituted alkyl, optionally substituted aryl and optionally substituted cycloalkyl.

Whenever used in the present invention the term “compounds of the invention” or a similar term is meant to include the compounds of general Formula I or II and any subgroup thereof. This term also refers to the compounds as depicted in Tables 1, 2, 3, 4, 5, 6 and 7 and their derivatives, N-oxides, salts, solvates, hydrates, stereoisomeric forms, racemic mixtures, tautomeric forms, optical isomers, analogues, pro-drugs, esters and metabolites, as well as their quaternized nitrogen analogues. The N-oxide forms of said compounds are meant to comprise compounds wherein one or several nitrogen atoms are oxidized to the so-called N-oxide.

As used in the specification and the appended claims, the singular forms “a”, “an,” and “the” include plural referents unless the context clearly dictates otherwise. By way of example, “a compound” means one compound or more than one compound.

The terms described above and others used in the specification are well understood to those in the art.

Preferred features of the compounds of this invention are now set forth.

In a particular embodiment, the compounds of Formula I or II are of Formula III, IV, V or VI

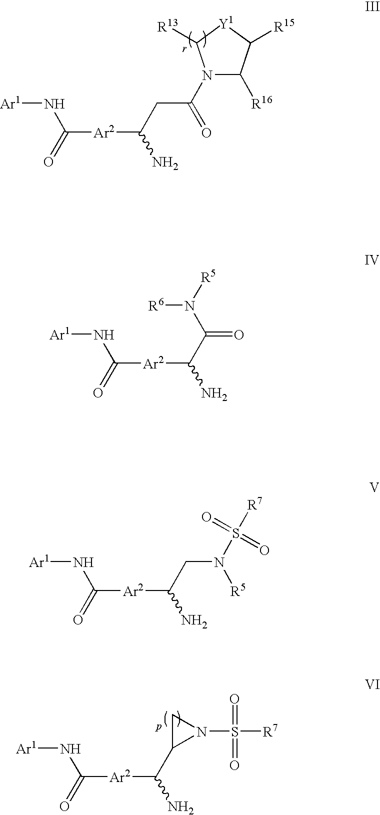

Y¹ is selected from —CH₂—, —CH(R¹⁴)—, —NH—, —O—, —S—, —C(═O)—,
p is selected from 2, 3 or 4, preferably 3 or 4, more preferably 3,
r is an integer selected from 0, 1, 2 or 3,
wherein R¹³ and R¹⁴ are each independently selected from hydrogen or alkyl,
or R¹³ and R¹⁴ form together with the carbon atoms to which they are attached form an aryl, a heteroaryl, a cycloalkyl or a heterocyclyl
or r is 2 and two R¹³ form together with the carbon atoms to which they are attached form an aryl, an heteroaryl, a cycloalkyl or a heterocyclyl,
wherein R¹⁵ and R¹⁶ together with the carbon atom to which they are attached form an aryl, a cycloalkyl, a heteroaryl a heterocyclyl, each optionally substituted with one or more substituent selected from halo, alkoxy, alkyl, alkylamino, alkylcarbonyl, alkylheteroaryl, alkylsuphonyl, aralkyl, aryl, arylamino, aryloxy, cyano, haloalkoxy, haloalkyl, heteroaryl, heteroarylalkyl, heteroarylcarbonyl, heterocyclyl, hydroxyl, nitro, oxo, or sulfonyl,
wherein Ar¹, Ar², R⁵, R⁶ and R⁷ have the same meaning as that defined above.

Ar¹ is, preferably, a 4-pyridyl ring which, may be optionally substituted, or comprises a 4-pyridyl ring as part of a bicyclic structure wherein such bicyclic structure is attached to the nitrogen atom of the amide moiety shown in Formula I or II through the (1) carbon atom in the 4-pyridyl ring.

Preferred structures for Ar¹ are of the Formula:

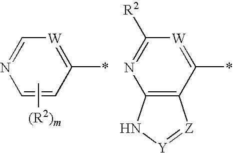

wherein
m is an integer selected from 0, 1, 2 or 3;

### W is C(R²) or N;

Y and Z are independently selected from the group comprising N and CR²;
R² is selected from hydrogen, halogen, or a group selected from alkyl, cycloalkyl, alkenyl, alkynyl, aryl or heteroaryl wherein each of said group is optionally substituted by one or more substituents selected from the group comprising halo, hydroxyl, amido, carboxy, amino, cyano, haloalkoxy, and haloalkyl.

In these preferred structures for Ar¹, the following features are preferred:

m is either 0 or 1, preferably 0; and
W is N or C(R²); particularly wherein the R² present in W is hydrogen.

In a particular embodiment, in these structures for Ar¹, the following features are preferred wherein Y is CH and Z is CH or wherein Y is CH and Z is N, or wherein Y is N and Z is CH.

Ar² is preferably of the Formula:

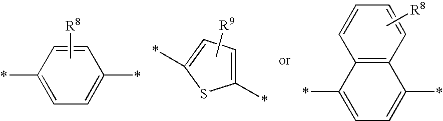

wherein
R⁸ is selected from the group comprising hydrogen and halogen, alkenyl, alkyl, alkynyl, acylamino, alkoxy, arylamino, nitro, haloalkoxy, aryl or heteroaryl, each of said group being optionally substituted by one or more substituents; and
R⁹ is selected from the group comprising hydrogen, halogen and alkyl.

Especially preferably, —Ar²— is either

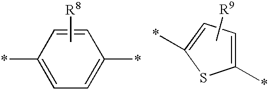

preferably wherein R⁸ and R⁹ are hydrogen.

Preferably, where A is present in R¹, A is oxygen or sulfur. In some embodiments, A is preferably sulfur. In other embodiments, A is preferably oxygen.

According to an embodiment of the present invention, preferred structures are compounds having one of the structural Formula

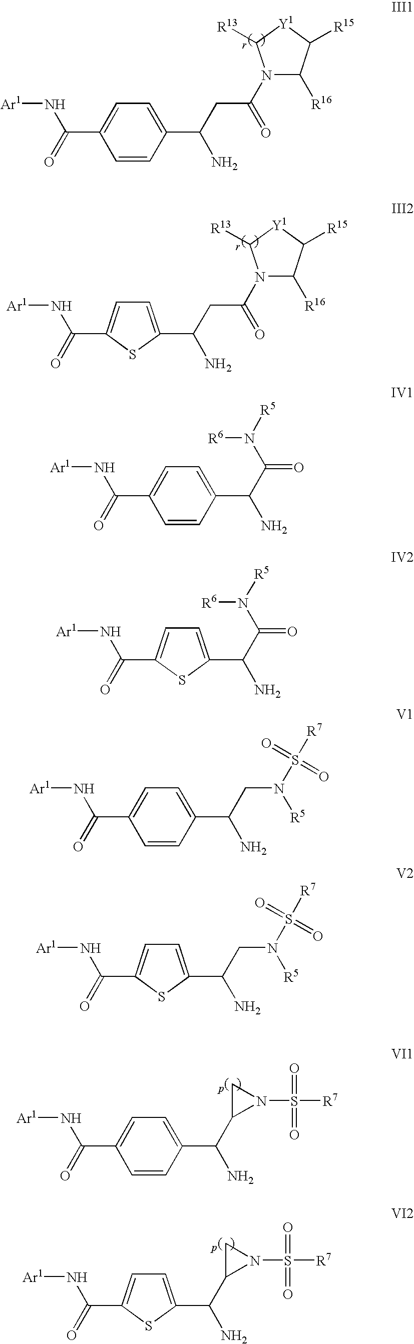

wherein Ar¹, R⁵, R⁶, R⁷, R¹³, R¹⁵, R¹⁶, r and p have the same meaning as that defined above.

According to a particular embodiment of the present invention, the compounds have one of the structural Formula

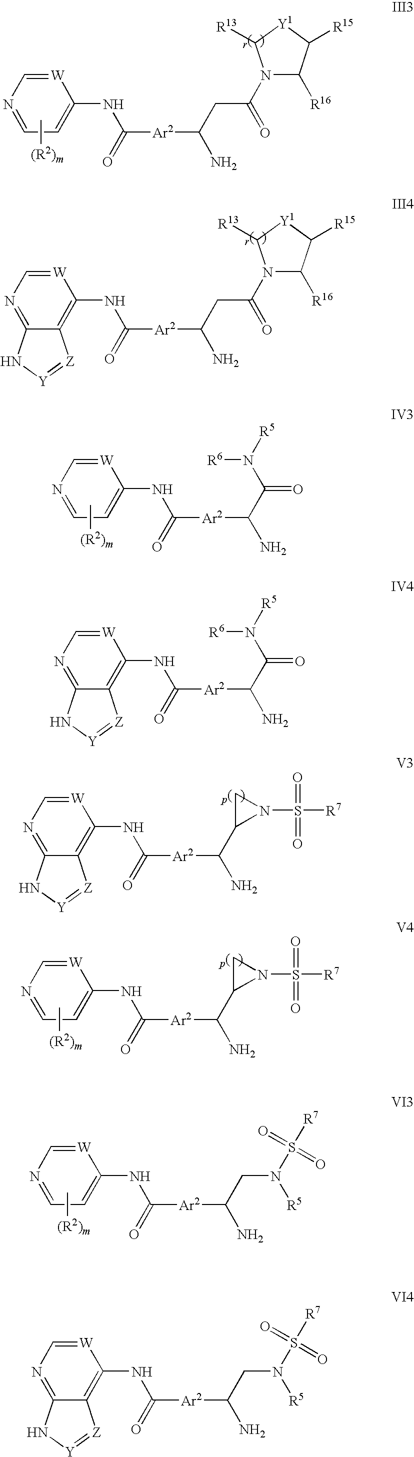

wherein Ar², W, Y, Z, Y¹, R², R⁵, R⁶, R⁷, R¹³, R¹⁵, R¹⁶, r, m and p have the same meaning as that defined above.

In an embodiment, Ar¹ is heteroaryl and R⁵, R⁶ are each independently selected from optionally substituted aryl, alkyl, heteroaryl, aralkyl, heteroarylalkyl, fused arylcycloalkyl, cycloalkylalkyl, cycloalkyl, heterocyclyl, heterocyclylalkyl, alkoxyalkyl, alkylsulphonylaminoalkyl, alkoxy, hydroxyalkyl, alkylaminoalkyl, arylalkylheterocyloalkyl, alkylamino wherein the substituent is independently one or more optionally substituted alkyl, aryl, heteroaryl, cycloalkyl, heterocyclyl, amino, amido, oxo, nitro, carboxy, cyano, haloalkoxy, hydroxyl, halo, alkoxy, hydroxyalkyl, alkoxyalkoxy, alkoxyalkyl, aminoalkyl or alkylamino,

or wherein R⁵ and R⁶ together with the N to which they are attached form a optionally substituted heterocyclyl, or heteroaryl to which may be fused one or more optionally substituted aryl, cycloalkyl, heteroaryl, heterocyclyl, wherein the substituent is independently one or more alkyl, aryl, aralkyl, heteroaryl, cycloalkyl, heterocyclyl, amino, amido, oxo, nitro, carboxy, cyano, haloalkoxy, hydroxyl, halo, alkoxy, hydroxyalkyl, alkoxyalkoxy, alkoxyalkyl, aminoalkyl or alkylamino; each substituent being optionally substituted by one or more alkyl, aryl, halo, alkoxy, haloalkoxy, heteroaryl, heteroarylalkyl, aralkyl, hydroxyl, hydroxyalkyl.

In another embodiment, Ar¹ is heteroaryl and R⁷ is selected from optionally substituted alkyl, heteroaryl, cycloalkyl, heterocyclyl, or aryl to which may be fused one or more optionally substituted aryl, cycloalkyl, heteroaryl, heterocyclyl, wherein the substituent is independently one or more, alkyl, aryl, heteroaryl, cycloalkyl, heterocyclyl, amino, amido, oxo, nitro, carboxy, cyano, haloalkoxy, hydroxyl, halo, alkoxy, hydroxyalkyl, alkoxyalkoxy, alkoxyalkyl, aminoalkyl or alkylamino. In a preferred embodiment, Ar¹ is pyridin-4-yl.

In an embodiment, the present invention provides compounds as defined above with Ar¹ being pyridin-4-yl, Ar² being phenylene and R⁵ being hydrogen and R⁶ being selected from phenylmethyl, 2-phenylmethyl, 1-phenylethyl, 2-phenylpropyl, 2(2-chlorophenyl)methyl, 2(3-chlorophenyl)methyl, 2(4-chlorophenyl)methyl, 2(4-methoxyphenyl)methyl, 2(2-fluorophenyl)methyl, 2(3-fluorophenyl)methyl, 2(4-fluorophenyl)methyl and R⁷ is selected from methyl, ethyl, propyl, phenylmethyl, phenyl, 4-methylphenyl, 1-phenylethyl, 2-phenylpropyl, 2(2-chlorophenyl)methyl, 2(3-chlorophenyl)methyl, 2(4-chlorophenyl)methyl, 2(4-methoxyphenyl)methyl, 2(2-methoxyphenyl)methyl, 2(2-fluorophenyl)methyl, 2(3-fluorophenyl)methyl and 2(4-fluorophenyl)methyl.

In an embodiment, the present invention provides compounds as defined above with Ar¹ being pyridin-4-yl, Ar² being phenylene and wherein R⁵ and R⁶ together with the N to which they are attached form a optionally substituted heterocyclyl, or heteroaryl to which may be fused one or more optionally substituted aryl, cycloalkyl, heteroaryl, heterocyclyl, wherein the substituent is independently one or more optionally substituted alkyl, aryl, heteroaryl, cycloalkyl, heterocyclyl, amino, amido, oxo, nitro, carboxy, cyano, haloalkoxy, hydroxyl, halo, alkoxy, hydroxyalkyl, alkoxyalkoxy, alkoxyalkyl, aminoalkyl or alkylamino.

According to a particular embodiment of the present invention, the compounds have one of the structural Formula

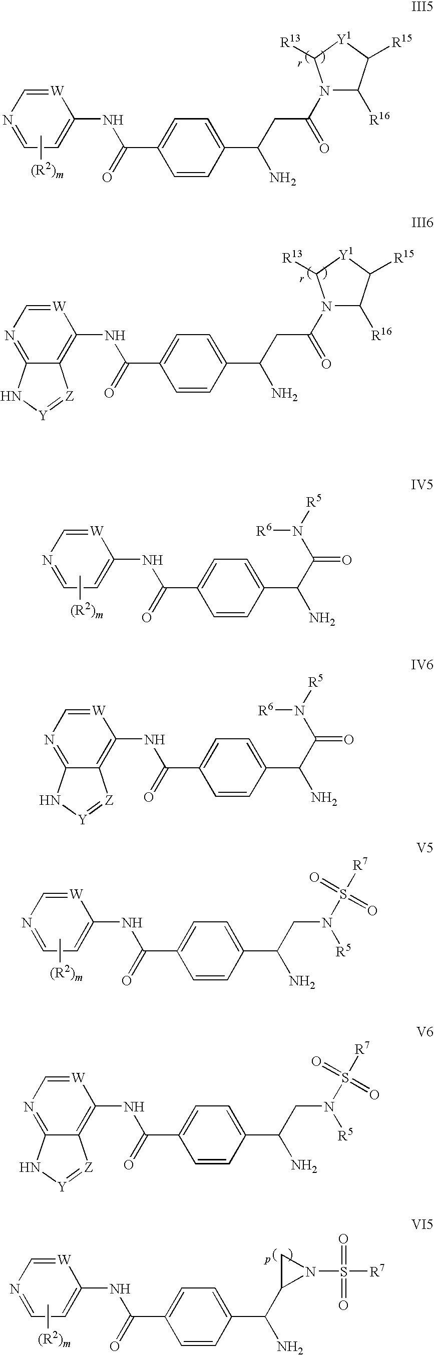

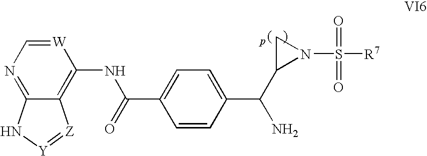

wherein W, Y, Z, Y¹, R², R⁵, R⁶, R⁷, R¹³, R¹⁵, R¹⁶, r, m and p have the same meaning as that defined above.

In an embodiment, the present invention provides compounds as defined above wherein R⁵ is selected from hydrogen, alkyl or cycloalkyl, and wherein W, Y, Z, Y¹, R², R⁶, R⁷, R¹³, R¹⁵, R¹⁶, r, m and p have the same meaning as that defined above.

In an embodiment, the present invention provides compounds as defined above wherein R⁶ is selected from alkyl, aryl, aralkyl, heteroaryl, heteroarylalkyl, cycloalkyl, cycloalkylalkyl, alkoxy, alkoxyalky, alkylsulphonylaminoalkyl, hydroxyalkyl, alkylaminoalkyl, heterocyclyl, heterocyclylalkyl, alkylamino, each being optionally substituted with one or more substituents selected from alkyl, alkoxy, halo, alkoxy, hydroxyl, hydroxyalkyl, aryl, heteroaryl or heterocyclyl, and wherein W, Y, Z, Y¹, R², R⁵, R⁷, R¹³, R¹⁵, R¹⁶, r, m and p have the same meaning as that defined above.

In an embodiment, the present invention provides compounds as defined above wherein R⁵ and R⁶ together with the N to which they are attached form a optionally substituted heterocyclyl or heteroaryl to which may be fused one or more optionally substituted aryl, cycloalkyl, heteroaryl, heterocyclyl, wherein the substituent is independently selected from one or more alkyl, aryl, heteroaryl, aralkyl, cycloalkyl, heterocyclyl, amino, amido, oxo, nitro, carboxy, cyano, haloalkoxy, hydroxyl, halo, alkoxy, hydroxyalkyl, alkoxyalkoxy, alkoxyalkyl, aminoalkyl or alkylamino; each substituent being optionally substituted by one or more alkyl, aryl, halo, alkoxy, haloalkoxy, heteroaryl, heteroarylalkyl, aralkyl, hydroxyl, hydroxyalkyl, and wherein W, Y, Z, Y¹, R², R⁷, R¹³, R¹⁵, R¹⁶, r, m and p have the same meaning as that defined above.

According to a particular embodiment of the present invention, the compounds have one of the structural Formula

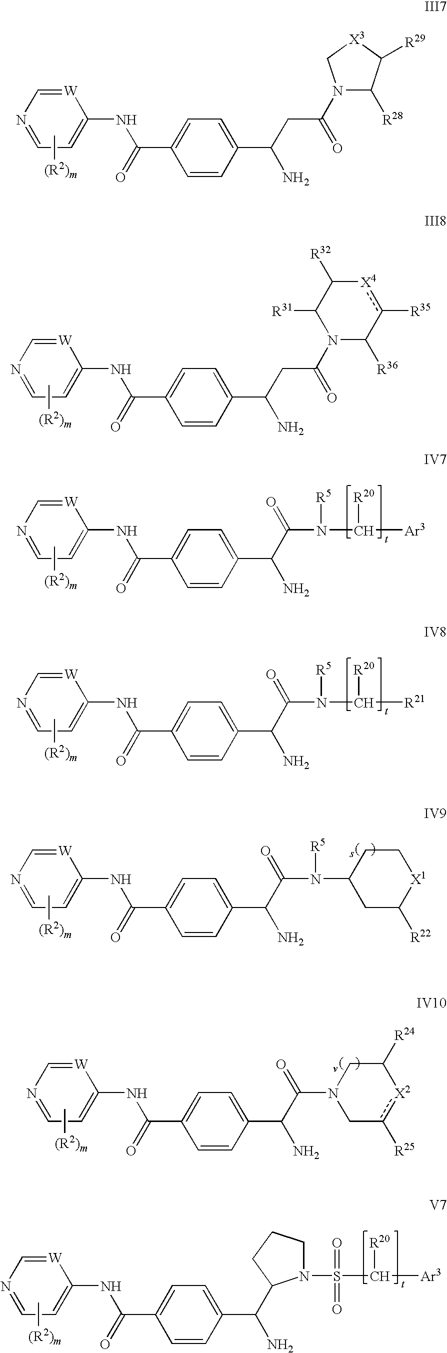

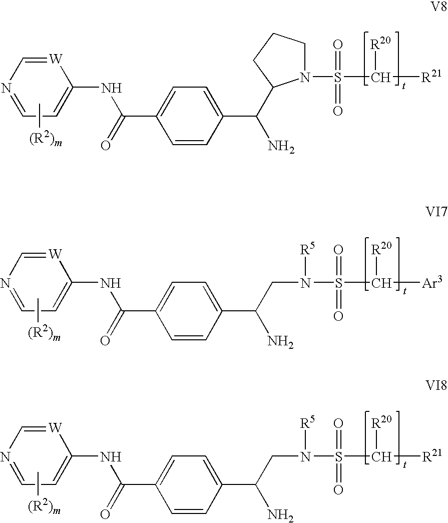

wherein
t is an integer selected from 0, 1, 2, 3, 4, 5, 6, 7 or 8,
s is an integer selected from 0 or 1,
v is an integer selected from 0 or 1,
R⁵ is selected from hydrogen, alkyl or cycloalkyl,
R²⁰ is selected from hydrogen or alkyl,
Ar³ is selected from aryl, heteroaryl, cycloalkyl, heterocyclyl, being each optionally substituted with one or more substituents selected from halo, alkoxy, alkyl, hydroxyalkyl, hydroxyl, aryl, aryloxy, aralkyl, heteroaryl, heteroarylalkyl,
R²¹ is selected from hydrogen, alkyl, cycloalkyl, alkoxy, alkylsulfonylamino, hydroxyl, alkylamino,
R²² is selected from hydrogen, alkyl, aryl,
X¹ is selected from CHR²³ or NR²³, wherein R²³ is selected from hydrogen, alkyl, aryl, aralkyl, heteroaryl, heteroarylalkyl, each being optionally substituted by one or more substituents selected from alkyl, aryl, aryloxy, aralkyl, halo, hydroxyl, alkoxy,
or wherein R²² and R²³ together with the carbon atoms to which they are attached form a optionally substituted aryl, heterocyclyl or heteroaryl, wherein the substituent is independently selected from one or more alkyl, aryl, heteroaryl, aralkyl, aryloxy, cycloalkyl, heterocyclyl, amino, amido, oxo, nitro, carboxy, cyano, haloalkoxy, hydroxyl, halo, alkoxy, hydroxyalkyl, alkoxyalkoxy, alkoxyalkyl, aminoalkyl or alkylamino;
X² is selected from O, NR²⁶ or C(R²⁶)R²⁷, wherein R²⁶ and R²⁷ are each independently hydrogen or are selected from the group consisting of alkyl, aryl, aralkyl, heteroaryl, hydroxyalkyl, alkoxy, hydroxyl, each group being optionally substituted by one or more alkyl, halo, aryl, aryloxy, aralkyl, alkoxy,
wherein R²⁴ and R²⁵ are each independently selected from hydrogen or are selected from the group consisting of alkyl, aryl, aralkyl, heteroaryl, hydroxyalkyl, alkoxy, hydroxyl, each group being optionally substituted by one or more alkyl, halo, aryl, aryloxy, aralkyl, alkoxy,
or wherein R²⁴ and R²⁶ together with the atoms to which they are attached form an optionally substituted aryl, heterocyclyl or heteroaryl, wherein the substituent is independently selected from one or more alkyl, aryl, heteroaryl, aralkyl, aryloxy, cycloalkyl, heterocyclyl, amino, amido, oxo, nitro, carboxy, cyano, haloalkoxy, hydroxyl, halo, alkoxy, hydroxyalkyl, alkoxyalkoxy, alkoxyalkyl, aminoalkyl or alkylamino;
X³ is selected from O, S, NR³⁰ or CHR³⁰ wherein R³⁰ is selected from hydrogen or a group selected from alkyl, hydroxyl, aryl, heteroaryl, hydroxyalkyl, heteroarylcarbonyl, aralkyl, each group being optionally substituted with one or more halo, alkyl, alkoxy, alkoxycarbonyl, aryl, heteroaryl,
wherein R²⁸ and R²⁹ are each independently selected from hydrogen or are selected from the group consisting of alkyl, hydroxyl, aryl, heteroaryl, hydroxyalkyl, heteroarylcarbonyl, aralkyl, each group being optionally substituted by one or more alkyl, halo, aryl, aryloxy, aralkyl, alkoxy,
or wherein R²⁹ and R³⁰ together with the atoms to which they are attached form an optionally substituted aryl, heterocyclyl or heteroaryl, wherein the substituent is independently selected from one or more alkyl, aryl, heteroaryl, aralkyl, aryloxy, cycloalkyl, heterocyclyl, amino, amido, oxo, nitro, carboxy, cyano, haloalkoxy, hydroxyl, halo, alkoxy, hydroxyalkyl, alkoxyalkoxy, alkoxyalkyl, aminoalkyl or alkylamino;
wherein X⁴ is selected from O, S, NR³³, C(R³³)R³⁴, wherein R³³ and R³⁴ are each independently hydrogen or selected from the group consisting of alkyl, hydroxyl, aryl, heteroaryl, hydroxyalkyl, heteroarylcarbonyl, aralkyl, each group being optionally substituted with one or more halo, alkyl, alkoxy, alkoxycarbonyl, aryl, heteroaryl,
wherein R³¹, R³², R³⁵ and R³⁶ are each independently selected from hydrogen or are selected from the group consisting of alkyl, hydroxyl, aryl, heteroaryl, hydroxyalkyl, heteroarylcarbonyl, aralkyl, each group being optionally substituted by one or more alkyl, halo, aryl, aryloxy, aralkyl, alkoxy,
or wherein R³¹ and R³² together with the atoms to which they are attached form an optionally substituted aryl, heterocyclyl or heteroaryl, wherein the substituent is independently selected from one or more alkyl, aryl, heteroaryl, aralkyl, aryloxy, cycloalkyl, heterocyclyl, amino, amido, oxo, nitro, carboxy, cyano, haloalkoxy, hydroxyl, halo, alkoxy, hydroxyalkyl, alkoxyalkoxy, alkoxyalkyl, aminoalkyl or alkylamino;
or wherein R³² and R³³ together with the atoms to which they are attached form an optionally substituted aryl, heterocyclyl or heteroaryl, wherein the substituent is independently selected from one or more alkyl, aryl, heteroaryl, aralkyl, aryloxy, cycloalkyl, heterocyclyl, amino, amido, oxo, nitro, carboxy, cyano, haloalkoxy, hydroxyl, halo, alkoxy, hydroxyalkyl, alkoxyalkoxy, alkoxyalkyl, aminoalkyl or alkylamino,
and wherein the dotted line represent an optional double bond.

In an embodiment, the present invention relates to any of the compounds described above wherein, Ar³ is selected from phenyl, biphenylyl, biphenylenyl, 5- or 6-tetralinyl, 1-, 2-, 3-, 4-, 5-, 6-, 7- or 8-azulenyl, 1- or 2-naphthyl, 1-, 2- or 3-indenyl, 1-, 2- or 9-anthryl, 1- 2-, 3-, 4- or 5-acenaphtylenyl, 3-, 4- or 5-acenaphtenyl, 1-, 2-, 3-, 4- or 10-phenanthryl, 1- or 2-pentalenyl, 1,2-, 3- or 4-fluorenyl, 4- or 5-indanyl, 5-, 6-, 7- or 8-tetrahydronaphthyl, 1,2,3,4-tetrahydronaphthyl, 1,4-dihydronaphthyl, dibenzo[a,d]cylcoheptenyl, 1-, 2-, 3-, 4- or 5-pyrenyl, 2- or 3-furyl, 2- or 3-thienyl, 1-, 2- or 3-pyrrolyl, 1-, 2-, 4- or 5-imidazolyl, 1-, 3-, 4- or 5-pyrazolyl, 3-, 4- or 5-isoxazolyl, 2-, 4- or 5-oxazolyl, 3-, 4- or 5-isothiazolyl, 2-, 4- or 5-thiazolyl, 1,2,3-triazol-1-, -2-, -4- or -5-yl, 1,2,4-triazol-1-, -3-, -4- or -5-yl, 1,2,3-oxadiazol-4- or -5-yl, 1,2,4-oxadiazol-3- or -5-yl, 1,2,5-oxadiazolyl, 1,3,4-oxadiazolyl, 1,2,3-thiadiazol-4- or -5-yl, 1,2,4-thiadiazol-3- or -5-yl, 1,2,5-thiadiazol-3- or -4-yl, 1,3,4-thiadiazolyl, 1- or 5-tetrazolyl, 2-, 3- or 4-pyridyl, 3- or 4-pyridazinyl, 2-, 4-, 5- or 6-pyrimidinyl, 2-, 3-, 4-, 5- 6-2H-thiopyranyl, 2-, 3- or 4-4H-thiopyranyl, 2-, 3-, 4-, 5-, 6- or 7-benzofuryl, 1-, 3-, 4- or 5-isobenzofuryl, 2-, 3-, 4-, 5-, 6- or 7-benzothienyl, 1-, 3-, 4- or 5-isobenzothienyl, 1-, 2-, 3-, 4-, 5-, 6- or 7-indolyl, 2- or 3-pyrazinyl, 1,4-oxazin-2- or -3-yl, 1,4-dioxin-2- or -3-yl, 1,4-thiazin-2- or -3-yl, 1,2,3-triazinyl, 1,2,4-triazinyl, 1,3,5-triazin-2-, -4- or -6-yl, thieno[2,3-b]furan-2-, -3-, -4-, or -5-yl, 1-, 2-, 4- or 5-benzimidazolyl, 1-, 3-, 4-, 5-, 6- or 7-benzopyrazolyl, 3-, 4-, 5-, 6- or 7-benzisoxazolyl, 2-, 4-, 5-, 6- or 7-benzoxazolyl, 3-, 4-, 5-, 6- or 7-benzisothiazolyl, 2-, 4-, 5-, 6- or 7-benzothiazolyl, 1-, 2-thianthrenyl, 3-, 4- or 5-isobenzofuranyl, 1-, 2-, 3-, 4- or 9-xanthenyl, 1-, 2-, 3- or 4-phenoxathiinyl, 2-, 3-pyrazinyl, 1-, 2-, 3-, 4-, 5-, 6-, 7- or 8-indolizinyl, 2-, 3-, 4- or 5-isoindolyl, 1-, 2-, 3-, 4-, 5-, 6- or 7-indazolyl, 2-, 6-, 7- or 8-purinyl, 4-, 5- or 6-phthalazinyl, 2-, 3- or 4-naphthyridinyl, 2-, 5- or 6-quinoxalinyl, 2-, 4-, 5-, 6-, 7- or 8-quinazolinyl, 1-, 2-, 3- or 4-quinolizinyl, 2-, 3-, 4-, 5-, 6-, 7-, or 8-quinolinyl(quinolyl), 2-, 4-, 5-, 6-, 7- or 8-quinazolyl, 1-, 3-, 4-, 5-, 6-, 7- or 8-isoquinolinyl(isoquinolyl), 3-, 4-, 5-, 6-, 7- or 8-cinnolinyl, 2-, 4-, 6- or 7-pteridinyl, 1-, 2-, 3-, 4- or 9-carbazolyl, 1-, 2-, 3-, 4-, 5-, 6-, 7-, 8- or 9-carbolinyl, 1-, 2-, 3-, 4-, 5-, 6-, 7-, 8-, 9- or 10-phenanthridinyl, 1-, 2-, 3- or 4-acridinyl, 1-, 2-, 3-, 4-, 5-, 6-, 7-, 8- or 9-perimidinyl, 2-, 3-, 4-, 5-, 6-, 7-, 8-, 9- or 10-(1,7)phenanthrolinyl, 1- or 2-phenazinyl, 1-, 2-, 3-, 4-, or 10-phenothiazinyl, 3- or 4-furazanyl, 1-, 2-, 3-, 4-, or 10-phenoxazinyl, cyclopropyl, cyclobutyl, cyclopentyl, cyclohexyl, cycloheptyl, cyclooctyl, cyclononyl, cyclodecyl, piperidinyl, azetidinyl, imidazolinyl, imidazolidinyl, isoxazolinyl, oxazolidinyl, isoxazolidinyl, thiazolidinyl, isothiazolidinyl, piperidyl, succinimidyl, 3H-indolyl, indolinyl, isoindolinyl, chromenyl, isochromanyl, xanthenyl, 2H-pyrrolyl, 1-pyrrolinyl, 2-pyrrolinyl, 3-pyrrolinyl, pyrrolidinyl, 4H-quinolizinyl, 4aH-carbazolyl, 2-oxopiperazinyl, piperazinyl, homopiperazinyl, 2-pyrazolinyl, 3-pyrazolinyl, pyranyl, dihydro-2H-pyranyl, 4H-pyranyl, 3,4-dihydro-2H-pyranyl, triazinyl, cinnolinyl, phthalazinyl, oxetanyl, thietanyl, 3-dioxolanyl, 1,4-dioxanyl, 2,5-dioximidazolidinyl, 2,2,4-piperidonyl, 2-oxopiperidinyl, 2-oxopyrrolodinyl, 2-oxoazepinyl, indolinyl, tetrahydropyranyl, tetrahydrofuranyl, tetrehydrothienyl, tetrahydroquinolinyl, tetrahydroisoquinolinyl, thiomorpholinyl, thiomorpholinyl sulfoxide, thiomorpholinyl sulfone, 1,3-dioxolanyl, 1,4-oxathianyl, 1,4-dithianyl, 1,3,5-trioxanyl, 6H-1,2,5-thiadiazinyl, 2H-1,5,2-dithiazinyl, 2H-oxocinyl, 1H-pyrrolizinyl, tetrahydro-1,1-dioxothienyl, N-formylpiperazinyl, morpholinyl, each being optionally substituted with one or more substituents selected from halo, alkoxy, haloalkyl, haloalkoxy, alkyl, hydroxyalkyl, hydroxyl, aryl, aryloxy, aralkyl, heteroaryl, or heteroarylalkyl,

It is clear to a person skilled in the art that the compounds of Formula I or II contain at least one asymmetric center and thus may exist as different stereoisomeric forms. This asymmetric center is indicated with an asterisk (*) in the figure below.

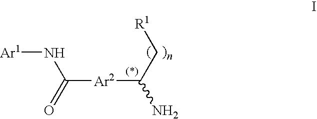

The absolute configuration of each asymmetric center that may be present in the compounds of Formula I or II may be indicated by the stereochemical descriptors R and S. when two chiral centers are present in the compound, in the configuration R,R for example the first letter refers to the configuration of the carbon bearing the amine group (*).

In a particular embodiment: for the compounds of Formula I or II preferred configuration has Formula Ia, when Ar² is phenylene of naphtlylene. Preferred configuration has Formula Ib, when Ar² is thienylene.

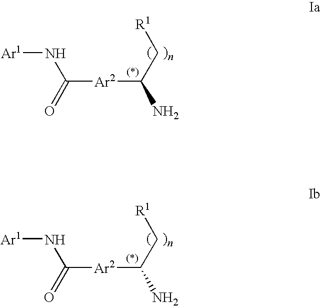

In a particular embodiment, for the compounds of Formula XVI, the carbon atom marked with the asterisk (*) preferably has the S configuration, i.e. compounds of Formula XVIa.

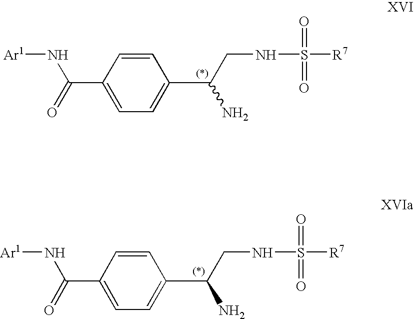

In a particular embodiment, for the compounds of Formula XIV, when R⁵ and/or R⁶ do not contain any chiral centers, the carbon atom marked with the asterisk (*) preferably has the S configuration, i.e. compounds of Formula XIVa.

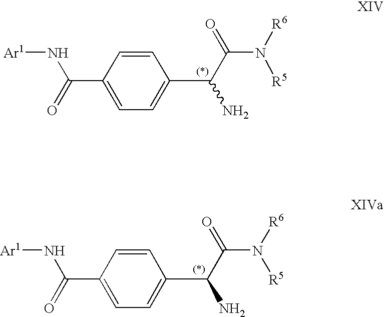

In compounds of Formula XIV, when R⁵ and/or R⁶ contain a chiral center, the carbon atom marked with the asterisk (*) preferably has the S configuration.

For example compound 83, which can have 4 diastereoisomers: 83a 4-{(S)-1-Amino-2-[(R)-2-(3-fluoro-phenyl)-pyrrolidin-1-yl]-2-oxo-ethyl}-N-pyridin-4-yl-benzamide; 83b 4-{(S)-1-Amino-2-[(S)-2-(3-fluoro-phenyl)-pyrrolidin-1-yl]-2-oxo-ethyl}-N-pyridin-4-yl-benzamide; 83c 4-{(R)-1-Amino-2-[(R)-2-(3-fluoro-phenyl)-pyrrolidin-1-yl]-2-oxo-ethyl}-N-pyridin-4-yl-benzamide; and 83d 4-{(R)-1-Amino-2-[(S)-2-(3-fluoro-phenyl)-pyrrolidin-1-yl]-2-oxo-ethyl}-N-pyridin-4-yl-benzamide.

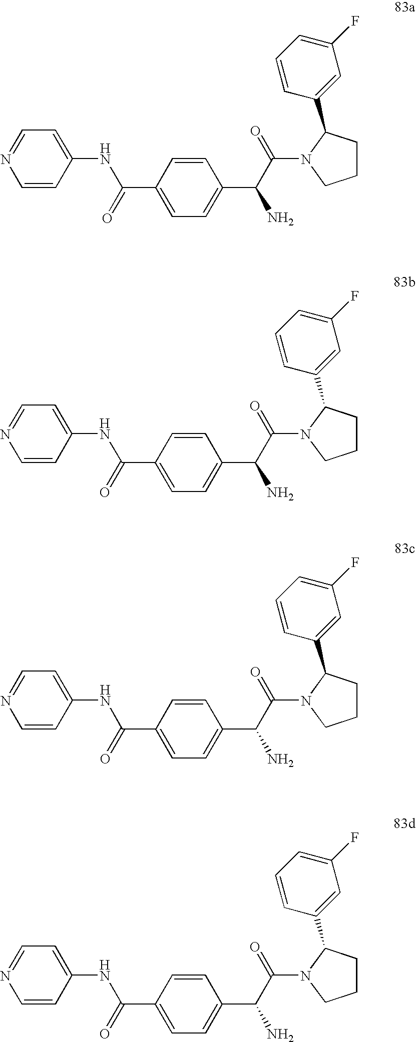

the carbon atom marked with the asterisk (*) (Compound 83e) preferably has the S configuration, i.e. preferred compounds are compounds 83a and 83b.

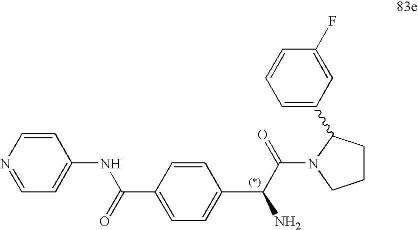

In a particular embodiment, for the compounds of Formula XV, when R⁵ and/or R⁶ do not contain any chiral centers, the carbon atom marked with the asterisk (*) preferably has the R configuration, i.e. compounds of Formula XVa.

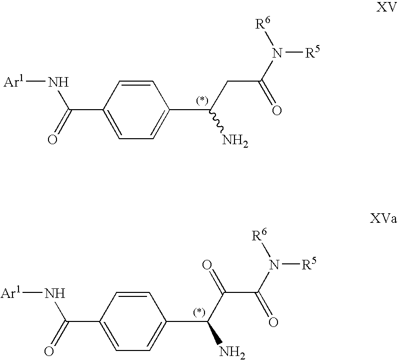

In compounds of Formula XV, when R⁵ and/or R⁶ contain a chiral center, the carbon atom marked with the asterisk (*) preferably has the R configuration.

For example compound 87, which can have 4 diastereoisomers: 87a 4-{(R)-1-Amino-3-[(R)-2-(3-fluoro-phenyl)-pyrrolidin-1-yl]-3-oxo-propyl}-N-pyridin-4-yl-benzamide; 87b 4-{(R)-1-Amino-3-[(S)-2-(3-fluoro-phenyl)-pyrrolidin-1-yl]-3-oxo-propyl}-N-pyridin-4-yl-benzamide; 87c 4-{(S)-1-Amino-3-[(S)-2-(3-fluoro-phenyl)-pyrrolidin-1-yl]-3-oxo-propyl}-N-pyridin-4-yl-benzamide; and 87d 4-{(S)-1-Amino-3-[(R)-2-(3-fluoro-phenyl)-pyrrolidin-1-yl]-3-oxo-propyl}-N-pyridin-4-yl-benzamide.

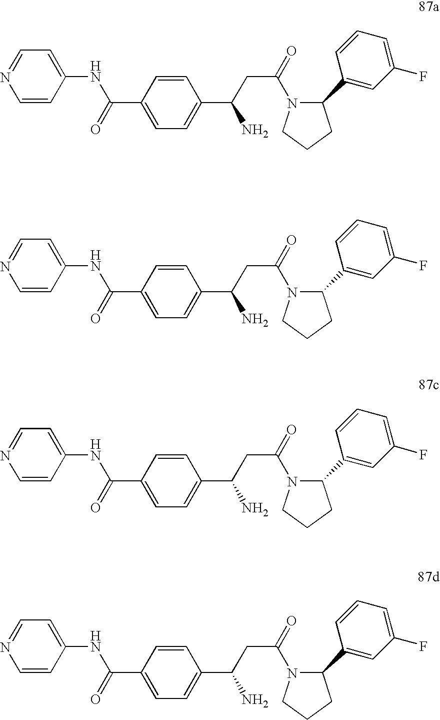

the carbon atom marked with the asterisk (*) (Compound 83e) preferably has the R configuration, i.e. preferred compounds are compounds 87a and 87b.

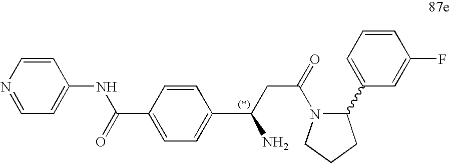

The compounds of the invention may be in the form of pharmaceutically and/or veterinary acceptable salts, as generally described below. Some preferred, but non-limiting examples of suitable pharmaceutically acceptable organic and/or inorganic acids are as hydrochloric acid, hydrobromic acid, sulfuric acid, nitric acid, acetic acid and citric acid, as well as other pharmaceutically acceptable acids known per se (for which reference is made to the prior art referred to below).

When the compounds of the invention contain an acidic group as well as a basic group the compounds of the invention may also form internal salts, and such compounds are within the scope of the invention. When the compounds of the invention contain a hydrogen-donating heteroatom (e.g. NH), the invention also covers salts and/or isomers formed by transfer of said hydrogen atom to a basic group or atom within the molecule.

In addition, although generally, with respect to the salts of the compounds of the invention, pharmaceutically acceptable salts are preferred, it should be noted that the invention in its broadest sense also included non-pharmaceutically acceptable salts, which may for example be used in the isolation and/or purification of the compounds of the invention. For example, salts formed with optically active acids or bases may be used to form diastereoisomeric salts that can facilitate the separation of optically active isomers of the compounds of Formula I or II above.

The invention also generally covers all pharmaceutically acceptable predrugs and prodrugs of the compounds of Formula I or II, for which general reference is made to the prior art cited hereinbelow.

The term “pro-drug” as used herein means the pharmacologically acceptable derivatives such as esters, amides and phosphates, such that the resulting in vivo biotransformation product of the derivative is the active drug. The reference by Goodman and Gilman (The Pharmacological Basis of Therapeutics, 8th Ed, McGraw-Hill, Int. Ed. 1992, “Biotransformation of Drugs”, p 13-15) describing pro-drugs generally is hereby incorporated. Pro-drugs of the compounds of the invention can be prepared by modifying functional groups present in said component in such a way that the modifications are cleaved, either in routine manipulation or in vivo, to the parent component. Typical examples of pro-drugs are described for instance in WO 99/33795, WO 99/33815, WO 99/33793 and WO 99/33792 all incorporated herein by reference. Pro-drugs are characterized by increased bio-availability and are readily metabolized into the active inhibitors in vivo. The term “pre-drug”, as used herein, means any compound that will be modified to form a drug species, wherein the modification may take place either inside or outside of the body, and either before or after the pre-drug reaches the area of the body where administration of the drug is indicated.

As described, above, some of the compounds of the invention may contain one or more asymmetric carbon atoms that serve as a chiral center, which may lead to different optical forms (e.g. enantiomers or diastereoisomers). The invention comprises all such optical forms in all possible configurations, as well as mixtures thereof.

More generally, from the above, it will be clear to the skilled person that the compounds of the invention may exist in the form of different isomers and/or tautomers, including but not limited to geometrical isomers, conformational isomers, E/Z-isomers, stereochemical isomers (i.e. enantiomers and diastereoisomers) and isomers that correspond to the presence of the same substituents on different positions of the rings present in the compounds of the invention. All such possible isomers, tautomers and mixtures thereof are included within the scope of the invention.

The compounds of Formula I or II may be prepared as described in the experimental section below using methods and chemistries with which those skilled in the art shall be familiar.

It will also be clear that when the desired compounds of the invention, and/or the starting materials, precursors and/or intermediates used in the preparation thereof, contain functional groups that are sensitive to the reaction conditions used in the preparation of the compounds of the invention (i.e. that would undergo undesired reactions under those conditions if they were not suitably protected) can be protected during said reaction with one or more suitable protective group, which protective group can then be suitably removed after either completion of said reaction and/or as a later or final step in the preparation of the compounds of the invention. Protected forms of the inventive compounds are included within the scope of the present invention. Suitable protective groups, as well as methods and conditions for inserting them and removing them, will be clear to the skilled person and are generally described in the standard handbooks of organic chemistry, such as Greene and Wuts, “Protective groups in organic synthesis”, 3^{rd} Edition, Wiley and Sons, 1999, which is incorporated herein by reference in its entirety. It will also be clear to the skilled person that compounds of the invention in which one or more functional groups have been protected with suitable functional groups can find use as intermediates in the production and/or synthesis of the compounds of the invention, and as such form a further aspect of the invention.

Generally, the compounds of the invention can be prepared from amine- or carboxylic acid-containing intermediates 1, 2, 3, 4, 5, 6 or 7 described hereinafter which may be reacted with complementary reactive molecules so as to form the desired compound. The intermediates and complementary reactive molecules are either commercially available or may be easily prepared by the skilled person.

The compounds of the invention may be used for the inhibition of kinases in vitro or in vivo, preferably in vitro, for modulating biological pathways and/or processes in which such kinases are involved; and/or to prevent and/or treat diseases or disorders in which such kinases, pathways and/or processes are involved.

According to one preferred, but non-limiting embodiment, the compounds of the invention may be used to inhibit (at least one isoform of) ROCK; and as such may be used for any purposes known per se for inhibitors of ROCK.

In the invention, particular preference is given to compounds of Formula I or II above that in the inhibition assay for ROCK described below inhibit ROCK with an IC₅₀ value of less than 100 μM, preferably less than 50 μM, more preferably less than 10 μM, preferably less than 5 μM, even more preferably less than 1 μM, preferably less than 0.1 μM, and in particular less than 10 nM, for example less than or 1 nM, as determined by a suitable assay, such as the assay used in the Examples below.

The present invention also relates to the use of the compounds of Formula I or II above in (the preparation of a composition for) inhibiting at least one kinase, in particular for inhibiting at least one isoform of ROCK, more in particular for inhibiting ROCK I and/or ROCK II isoforms. As used herein, the term “ROCKI” can also be referred as ROK-β, p160ROCK, or Rho-kinase β and the term “ROCKII” can also be referred as ROK-α or Rho-kinase α. Said inhibition may be effected in vitro and/or in vivo, and when effected in vivo, is preferably effected in a selective manner, as defined above.

According to an embodiment, the invention provides a method for treating or lessening the severity of a ROCK-mediated disease or condition in a patient comprising the step of administering to said patient a compound according to the present invention.

The term “ROCK-mediated condition” or “disease”, as used herein, means any disease or other deleterious condition in which is known to play a role. The term “ROCK-mediated condition” or “disease” also means those diseases or conditions that are alleviated by treatment with a ROCK inhibitor. Accordingly, another embodiment of the present invention relates to treating or lessening the severity of one or more diseases in which ROCK is known to play a role.

According to particularly preferred embodiments, the compounds of the invention are preferably used in the prevention and/or treatment of at least one disease or disorder, preferably in which at least one isoform of ROCK is involved. According to an even more particularly preferred embodiment, the compounds of the invention may be used in the prevention and/or treatment of at least one disease or disorder in which the ROCK I or ROCK II is involved, such as, such as inflammatory diseases, chronic obstructive bladder disease (COBD) and the related erectile dysfunction as well as in diabetes related ED.

Specifically, the present invention relates to the use of a compound according to the invention for the preparation of a medicament for treating or lessening the severity of a disease or condition selected from eye disease or disorder (such as but not limited to retinopathy, glaucoma and degenerative retinal diseases such as macular degeneration and retinitis pigmentosa), kidney disease (such as but not limited to renal dysfunction), and bladder dysfunction (such as but not limited to chronic obstructive bladder disease), erectile dysfunction (such as but not limited to bladder disease related erectile dysfunction and diabetes related erectile dysfunction) neurological and CNS (brain) disease or disorder (such as but not limited to Alzheimer, meningitis and convulsions), hypertension, lung disease (such as but not limited to asthma, fibrosis, pneumonia, cystic fibrosis and respiratory distress syndrome), premature birth, cancer (such as but not limited to cancer of the brain (gliomas), breast, colon, head and neck, prostate, kidney, lung, intestine, nerve, skin, pancreas, liver, uterus, ovary, brain, thyroid gland; leukemia; lymphoma and melanoma), cardiovascular and vascular (blood vessel, artery) disease or disorder (such as but not limited to cerebrovascular contraction, ischemia, reperfusion, pulmonary vasoconstriction, acute stroke, congestive heart failure, cardiovascular ischemia, heart disease, cardiac remodeling, hypoxia peripheral circulation disorder, atherosclerosis, thrombosis, aneurism and hemorrhage), blood disease (such as but not limited to sepsis, eosinophilia, endotoxemia), musculoskeletal disease (such as but not limited to spasm), inflammatory disease, infection, allergy and autoimmune diseases or disorders, AIDS, bone disease (such as but not limited to osteoporosis), inflammatory diseases, diabetes (such as but not limited to hyperglycemia), obesity and pancreas disease.

For example, the compounds of the invention may be used in the prevention and/or treatment of diseases and disorders such as:

Cardiovascular and vascular diseases: including but not limited to acute stroke, congestive heart failure, cardiovascular ischemia, heart disease, cardiac remodeling, angina, coronary vasospasm, cerebral vasospasm, restenosis, hypertension, (pulmonary) hypertension, pulmonary vasoconstriction, arteriosclerosis, thrombosis (including deep thrombosis) and platelet related diseases.
Neurological and CNS disorders: including but not limited to stroke, multiple sclerosis, brain or spinal cord injury, inflammatory and demyelinating diseases such as Alzheimer's disease, MS and neuropathic pain. The present compounds are therefore suitable for preventing neurodegeneration and stimulating neurogeneration in various neurological disorders.
Proliferative diseases: such as cancer including but not limited to cancer of the brain (gliomas), breast, colon, intestine, skin, head and neck, kidney, lung, liver, ovarian, pancreatic, prostate or thyroid ; leukemia; sarcoma; lymphoma; and melanoma.
Inflammatory diseases: including but not limited to contact dermatitis, psoriasis, rheumatoid arthritis, inflammatory bowel disease, Crohn's disease and ulcerative colitis.

In addition, the compounds of the invention may be used in the prevention and/or treatment of diseases and disorders such as erectile dysfunction, bronchial asthma, osteoporosis, eye diseases such as glaucoma, macular degeneration and retinopathy, renal diseases and AIDS.

The present invention therefore relates to a method of treating or lessening the severity of a disease or condition selected from cardiovascular and vascular diseases including but not limited to acute stroke, congestive heart failure, cardiovascular ischemia, heart disease, cardiac remodeling, angina, coronary vasospasm, cerebral vasospasm, pulmonary vasoconstriction, restenosis, hypertension, (pulmonary) hypertension, arteriosclerosis, thrombosis (including deep thrombosis) and platelet related diseases; neurological and CNS disorders: including but not limited to stroke, multiple sclerosis, spinal or brain cord injury, brain or spinal cord injury, inflammatory and demyelinating diseases such as Alzheimer's disease, MS and neuropathic pain; proliferative diseases such as cancer including but not limited to cancer of the brain (gliomas), breast, colon, intestine, skin, head and neck, kidney, lung, liver, ovarian, pancreatic, prostate or thyroid; leukemia; sarcoma; lymphoma; melanoma; erectile dysfunction; bronchial asthma; osteoporosis; eye diseases such as glaucoma, macular degeneration and retinopathy; renal diseases; AIDS; preterm labor; vascular smooth muscle cell proliferation; myocardial hypertrophy; malignoma; ischemia/reperfusion-induced injury; endothelial dysfunction, Crohn's Disease and colitis; neurite outgrowth; Raynaud's Disease; benign prostatic hyperplasia; and atherosclerosis, wherein said method comprises administering to a patient in need thereof a compound or a composition according to the present invention.

For pharmaceutical use, the compounds of the invention may be used as a free acid or base, and/or in the form of a pharmaceutically acceptable acid-addition and/or base-addition salt (e.g. obtained with non-toxic organic or inorganic acid or base), in the form of a hydrate, solvate and/or complex, and/or in the form or a pro-drug or pre-drug, such as an ester. As used herein and unless otherwise stated, the term “solvate” includes any combination which may be formed by a compound of this invention with a suitable inorganic solvent (e.g. hydrates) or organic solvent, such as but not limited to alcohols, ketones, esters and the like. Such salts, hydrates, solvates, etc. and the preparation thereof will be clear to the skilled person; reference is for instance made to the salts, hydrates, solvates, etc. described in U.S. Pat. No. 6,372,778, U.S. Pat. No. 6,369,086, U.S. Pat. No. 6,369,087 and U.S. Pat. No. 6,372,733.

The pharmaceutically acceptable salts of the compounds according to the invention, i.e. in the form of water-, oil-soluble, or dispersible products, include the conventional non-toxic salts or the quaternary ammonium salts which are formed, e.g., from inorganic or organic acids or bases. Examples of such acid addition salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalene-sulfonate, nicotinate, oxalate, pamoate, pectinate, persulfate, 3-phenylpropionate, picrate, pivalate, propionate, succinate, tartrate, thiocyanate, tosylate, and undecanoate. Base salts include ammonium salts, alkali metal salts such as sodium and potassium salts, alkaline earth metal salts such as calcium and magnesium salts, salts with organic bases such as dicyclohexylamine salts, N-methyl-D-glucamine, and salts with amino acids such as arginine, lysine, and so forth. In addition, the basic nitrogen-containing groups may be quaternized with such agents as lower alkyl halides, such as methyl, ethyl, propyl, and butyl chloride, bromides and iodides; dialkyl sulfates like dimethyl, diethyl, dibutyl; and diamyl sulfates, long chain halides such as decyl, lauryl, myristyl and stearyl chlorides, bromides and iodides, aralkyl halides like benzyl and phenethyl-bromides and others. Other pharmaceutically acceptable salts include the sulfate salt ethanolate and sulfate salts.

Generally, for pharmaceutical use, the compounds of the inventions may be formulated as a pharmaceutical preparation comprising at least one compound of the invention and at least one pharmaceutically acceptable carrier, diluent or excipient and/or adjuvant, and optionally one or more further pharmaceutically active compounds.

By means of non-limiting examples, such a formulation may be in a form suitable for oral administration, for parenteral administration (such as by intravenous, intramuscular or subcutaneous injection or intravenous infusion), for topical administration (including ocular), for administration by inhalation, by a skin patch, by an implant, by a suppository, etc. Such suitable administration forms—which may be solid, semi-solid or liquid, depending on the manner of administration—as well as methods and carriers, diluents and excipients for use in the preparation thereof, will be clear to the skilled person; reference is again made to for instance U.S. Pat. No. 6,372,778, U.S. Pat. No. 6,369,086, U.S. Pat. No. 6,369,087 and U.S. Pat. No. 6,372,733, as well as to the standard handbooks, such as the latest edition of Remington's Pharmaceutical Sciences.

Some preferred, but non-limiting examples of such preparations include tablets, pills, powders, lozenges, sachets, cachets, elixirs, suspensions, emulsions, solutions, syrups, aerosols, ointments, creams, lotions, soft and hard gelatin capsules, suppositories, eye drops, sterile injectable solutions and sterile packaged powders (which are usually reconstituted prior to use) for administration as a bolus and/or for continuous administration, which may be formulated with carriers, excipients, and diluents that are suitable per se for such formulations, such as lactose, dextrose, sucrose, sorbitol, mannitol, starches, gum acacia, calcium phosphate, alginates, tragacanth, gelatin, calcium silicate, microcrystalline cellulose, polyvinylpyrrolidone, polyethylene glycol, cellulose, (sterile) water, methylcellulose, methyl- and propylhydroxybenzoates, talc, magnesium stearate, edible oils, vegetable oils and mineral oils or suitable mixtures thereof. The formulations can optionally contain other pharmaceutically active substances (which may or may not lead to a synergistic effect with the compounds of the invention) and other substances that are commonly used in pharmaceutical formulations, such as lubricating agents, wetting agents, emulsifying and suspending agents, dispersing agents, desintegrants, bulking agents, fillers, preserving agents, sweetening agents, flavoring agents, flow regulators, release agents, etc. The compositions may also be formulated so as to provide rapid, sustained or delayed release of the active compound(s) contained therein, for example using liposomes or hydrophilic polymeric matrices based on natural gels or synthetic polymers. In order to enhance the solubility and/or the stability of the compounds of a pharmaceutical composition according to the invention, it can be advantageous to employ α-, β- or γ-cyclodextrins or their derivatives. In addition, co-solvents such as alcohols may improve the solubility and/or the stability of the compounds. In the preparation of aqueous compositions, addition of salts of the compounds of the invention can be more suitable due to their increased water solubility.

Appropriate cyclodextrins are α-, β- or γ-cyclodextrins (CDs) or ethers and mixed ethers thereof wherein one or more of the hydroxyl groups of the anhydroglucose units of the cyclodextrin are substituted with alkyl, particularly methyl, ethyl or isopropyl, e.g. randomly methylated β-CD; hydroxyalkyl, particularly hydroxyethyl, hydroxypropyl or hydroxybutyl; carboxyalkyl, particularly carboxymethyl or carboxyethyl; alkylcarbonyl, particularly acetyl; alkoxycarbonylalkyl or carboxyalkoxyalkyl, particularly carboxymethoxypropyl or carboxyethoxypropyl; alkylcarbonyloxyalkyl, particularly 2-acetyloxypropyl. Especially noteworthy as complexants and/or solubilizers are β-CD, randomly methylated β-CD, 2,6-dimethyl-β-CD, 2-hydroxyethyl-β-CD, 2-hydroxyethyl-γ-CD, 2-hydroxypropyl-γ-CD and (2-carboxymethoxy)propyl-β-CD, and in particular 2-hydroxypropyl-β-CD (2-HP-β-CD). The term mixed ether denotes cyclodextrin derivatives wherein at least two cyclodextrin hydroxyl groups are etherified with different groups such as, for example, hydroxypropyl and hydroxyethyl. An interesting way of formulating the compounds in combination with a cyclodextrin or a derivative thereof has been described in EP-A-721,331. Although the formulations described therein are with antifungal active ingredients, they are equally interesting for formulating the compounds. Said formulations may also be rendered more palatable by adding pharmaceutically acceptable sweeteners and/or flavors. In particular, the present invention encompasses a pharmaceutical composition comprising an effective amount of a compound according to the invention with a pharmaceutically acceptable cyclodextrin. The present invention also encompasses cyclodextrin complexes consisting of a compound according to the invention and a cyclodextrin.

Particular reference is made to the compositions, formulations (and carriers, excipients, diluents, etc. for use therein), routes of administration etc., which are known per se for analogous pyridinocarboxamides, such as those described in U.S. Pat. No. 4,997,834 and EP-A-0 370 498.

For the treatment of pain, the compounds of the invention may be used locally or systemically. For local administration, the compounds may advantageously be used in the form of a spray, ointment or transdermal patch or another suitable form for topical, transdermal and/or intradermal administration; and for systemic administration, the compounds of the invention may advantageously be administered orally.

For ophthalmic application, solutions, gels, tablets and the like are often prepared using a physiological saline solution, gel or excipient as a major vehicle. Ophthalmic formulations should preferably be prepared at a comfortable pH with an appropriate buffer system.

More in particular, the compositions may be formulated in a pharmaceutical formulation comprising a therapeutically effective amount of particles consisting of a solid dispersion of the compounds of the invention and one or more pharmaceutically acceptable water-soluble polymers.

The term “a solid dispersion” defines a system in a solid state (as opposed to a liquid or gaseous state) comprising at least two components, wherein one component is dispersed more or less evenly throughout the other component or components. When said dispersion of the components is such that the system is chemically and physically uniform or homogenous throughout or consists of one phase as defined in thermodynamics, such a solid dispersion is referred to as “a solid solution”. Solid solutions are preferred physical systems because the components therein are usually readily bioavailable to the organisms to which they are administered. The term “a solid dispersion” also comprises dispersions that are less homogenous throughout than solid solutions. Such dispersions are not chemically and physically uniform throughout or comprise more than one phase.

The water-soluble polymer is conveniently a polymer that has an apparent viscosity of 1 to 100 mPa·s when dissolved in a 2% aqueous solution at 20° C. solution. Preferred water-soluble polymers are hydroxypropyl methylcelluloses or HPMC. HPMC having a methoxy degree of substitution from about 0.8 to about 2.5 and a hydroxypropyl molar substitution from about 0.05 to about 3.0 are generally water soluble. Methoxy degree of substitution refers to the average number of methyl ether groups present per anhydroglucose unit of the cellulose molecule. Hydroxy-propyl molar substitution refers to the average number of moles of propylene oxide which have reacted with each anhydroglucose unit of the cellulose molecule.

It may further be convenient to formulate the compounds in the form of nanoparticles which have a surface modifier adsorbed on the surface thereof in an amount sufficient to maintain an effective average particle size of less than 1000 nm. Suitable surface modifiers can preferably be selected from known organic and inorganic pharmaceutical excipients. Such excipients include various polymers, low molecular weight oligomers, natural products and surfactants. Preferred surface modifiers include nonionic and anionic surfactants.

Yet another interesting way of formulating the compounds according to the invention involves a pharmaceutical composition whereby the compounds are incorporated in hydrophilic polymers and applying this mixture as a coat film over many small beads, thus yielding a composition with good bio-availability which can conveniently be manufactured and which is suitable for preparing pharmaceutical dosage forms for oral administration. Said beads comprise (a) a central, rounded or spherical core, (b) a coating film of a hydrophilic polymer and an antiretroviral agent and (c) a seal-coating polymer layer. Materials suitable for use as cores in the beads are manifold, provided that said materials are pharmaceutically acceptable and have appropriate dimensions and firmness. Examples of such materials are polymers, inorganic substances, organic substances, and saccharides and derivatives thereof.

The preparations may be prepared in a manner known per se, which usually involves mixing at least one compound according to the invention with the one or more pharmaceutically acceptable carriers, and, if desired, in combination with other pharmaceutical active compounds, when necessary under aseptic conditions. Reference is again made to U.S. Pat. No. 6,372,778, U.S. Pat. No. 6,369,086, U.S. Pat. No. 6,369,087 and U.S. Pat. No. 6,372,733 and the further prior art mentioned above, as well as to the standard handbooks, such as the latest edition of Remington's Pharmaceutical Sciences.

The pharmaceutical preparations of the invention are preferably in a unit dosage form, and may be suitably packaged, for example in a box, blister, vial, bottle, sachet, ampoule or in any other suitable single-dose or multi-dose holder or container (which may be properly labeled); optionally with one or more leaflets containing product information and/or instructions for use. Generally, such unit dosages will contain between 1 and 1000 mg, and usually between 5 and 500 mg, of the at least one compound of the invention, e.g. about 10, 25, 50, 100, 200, 300 or 400 mg per unit dosage.

The compounds can be administered by a variety of routes including the oral, rectal, ocular, transdermal, subcutaneous, intravenous, intramuscular or intranasal routes, depending mainly on the specific preparation used and the condition to be treated or prevented, and with oral and intravenous administration usually being preferred. The at least one compound of the invention will generally be administered in an “effective amount”, by which is meant any amount of a compound of the Formula I or II above that, upon suitable administration, is sufficient to achieve the desired therapeutic or prophylactic effect in the individual to which it is administered. Usually, depending on the condition to be prevented or treated and the route of administration, such an effective amount will usually be between 0.01 to 1000 mg per kilogram body weight day of the patient per day, more often between 0.1 and 500 mg, such as between 1 and 250 mg, for example about 5, 10, 20, 50, 100, 150, 200 or 250 mg, per kilogram body weight day of the patient per day, which may be administered as a single daily dose, divided over one or more daily doses, or essentially continuously, e.g. using a drip infusion. The amount(s) to be administered, the route of administration and the further treatment regimen may be determined by the treating clinician, depending on factors such as the age, gender and general condition of the patient and the nature and severity of the disease/symptoms to be treated. Reference is again made to U.S. Pat. No. 6,372,778,U.S. Pat. No. 6,369,086, U.S. Pat. No. 6,369,087 and U.S. Pat. No. 6,372,733 and the further prior art mentioned above, as well as to the standard handbooks, such as the latest edition of Remington's Pharmaceutical Sciences.

Thus, in a further aspect, the invention relates to a composition, and in particular a composition for pharmaceutical use, that contains at least one compound of the invention (i.e. a compound that has been identified, discovered and/or developed using a nematode or method as described herein) and at least one suitable carrier (i.e. a carrier suitable for pharmaceutical use). The invention also relates to the use of a compound of the invention in the preparation of such a composition.

In accordance with the method of the present invention, said pharmaceutical composition can be administered separately at different times during the course of therapy or concurrently in divided or single combination forms. The present invention is therefore to be understood as embracing all such regimes of simultaneous or alternating treatment and the term “administering” is to be interpreted accordingly.

For an oral administration form, the compositions of the present invention can be mixed with suitable additives, such as excipients, stabilizers or inert diluents, and brought by means of the customary methods into the suitable administration forms, such as tablets, coated tablets, hard capsules, aqueous, alcoholic, or oily solutions. Examples of suitable inert carriers are gum arabic, magnesia, magnesium carbonate, potassium phosphate, lactose, glucose, or starch, in particular, corn starch. In this case, the preparation can be carried out both as dry and as moist granules. Suitable oily excipients or solvents are vegetable or animal oils, such as sunflower oil or cod liver oil. Suitable solvents for aqueous or alcoholic solutions are water, ethanol, sugar solutions, or mixtures thereof. Polyethylene glycols and polypropylene glycols are also useful as further auxiliaries for other administration forms. As immediate release tablets, these compositions may contain microcrystalline cellulose, dicalcium phosphate, starch, magnesium stearate and lactose and/or other excipients, binders, extenders, disintegrants, diluents and lubricants known in the art.

When administered by nasal aerosol or inhalation, these compositions may be prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. Suitable pharmaceutical formulations for administration in the form of aerosols or sprays are, for example, solutions, suspensions or emulsions of the compounds of the invention or their physiologically tolerable salts in a pharmaceutically acceptable solvent, such as ethanol or water, or a mixture of such solvents. If required, the formulation can also additionally contain other pharmaceutical auxiliaries such as surfactants, emulsifiers and stabilizers as well as a propellant.

For subcutaneous or intravenous administration, the compound according to the invention, if desired with the substances customary therefore such as solubilizers, emulsifiers or further auxiliaries are brought into solution, suspension, or emulsion. The compounds of the invention can also be lyophilized and the lyophilizates obtained used, for example, for the production of injection or infusion preparations. Suitable solvents are, for example, water, physiological saline solution or alcohols, e.g. ethanol, propanol, glycerol, in addition also sugar solutions such as glucose or mannitol solutions, or alternatively mixtures of the various solvents mentioned. The injectable solutions or suspensions may be formulated according to known art, using suitable non-toxic, parenterally-acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid.

When rectally administered in the form of suppositories, these formulations may be prepared by mixing the compounds according to the invention with a suitable non-irritating excipient, such as cocoa butter, synthetic glyceride esters or polyethylene glycols, which are solid at ordinary temperatures, but liquefy and/or dissolve in the rectal cavity to release the drug.

The compositions are of value in the veterinary field, which for the purposes herein not only includes the prevention and/or treatment of diseases in animals, but also—for economically important animals such as cattle, pigs, sheep, chicken, fish, etc.—enhancing the growth and/or weight of the animal and/or the amount and/or the quality of the meat or other products obtained from the animal. Thus, in a further aspect, the invention relates to a composition for veterinary use that contains at least one compound of the invention (e.g. a compound that has been identified, discovered and/or developed using a nematode or method as described herein) and at least one suitable carrier (i.e. a carrier suitable for veterinary use). The invention also relates to the use of a compound of the invention in the preparation of such a composition.

The invention will now be illustrated by means of the following synthetic and biological examples, which do not limited the scope of the invention in any way.

### EXAMPLES

### Example 1

Unless indicated otherwise, the purity of the compounds was confirmed by liquid chromatography/mass spectrometry (LC/MS), as follows:
HPLC system: Waters 2690 with photodiode array detector Waters 996; Column: C18; Gradient: solvent A (H₂O/formic acid 26.5 nM) 0%, to solvent B (CH₃CN/formic acid 17 nM) 80% in 3 min. Flow: 2.75 ml/min.
Mass spectrometer: Micromass Platform LC. Ionization: electrospray (polarity: negative and positive).

NMR spectra were determined on a Varian Mercury 300 MHz NMR using the indicated solvent as an internal reference. Melting points were determined on a Büchi B-540 and are non-corrected. All reagents used either were obtained commercially or were prepared in a manner known per se.

The following intermediates and general procedures were used to prepare the compounds described herein.

### Intermediates

### Intermediate 1: tert-butoxycarbonylamino-[4-(pyridin-4-ylcarbamoyl)-phenyl]-acetic acid

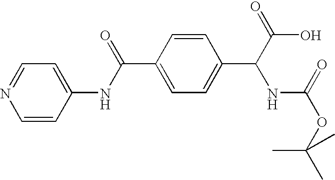

Amino-(4-bromo-phenyl)-acetic acid (3 g) was suspended in a mixture of concentrated HCl (10 ml) and MeOH (10 ml). The reaction mixture was stirred at room temperature (RT) for 2 days. The white precipitate was filtered off and dried. Amino-(4-bromo-phenyl)-acetic acid methyl ester was obtained as a white powder (49% yield).

Amino-(4-bromo-phenyl)-acetic acid methyl ester (1.8 g) was suspended in acetone (15 ml). 1M Na₂CO₃ (15 ml) was added followed by BOC₂O (1.1 eq). The mixture was stirred at RT for 4 days.

The precipitate was filtered off. The (4-bromo-phenyl)-tert-butoxycarbonylamino-acetic acid methyl ester was recrystallized from ethyl acetate (colorless solid, 38% yield).

The (4-bromo-phenyl)-tert-butoxycarbonylamino-acetic acid methyl ester (3.45 g) was dissolved in THF (52 ml) and water (8 ml). Potassium acetate (1 eq), 1,3-bis-diphenylphosphinopropane (0.02 eq) and Pd(OAc)₂ (0.04 eq) were added. The mixture was stirred under 50 atm of carbon monoxyde at 150° C. for 2 hours. The reaction mixture was cooled down at RT and then filtered and dried over MgSO₄. The solvent was removed under reduced pressure. The residue was purified by flash chromatography (DCM/MeOH:95/5 with 0.5% HCOOH) to yield the 4-(tert-butoxycarbonylamino-methoxycarbonyl-methyl-benzoic acid as a pale yellow powder (57% yield).

The 4-(tert-butoxycarbonylamino-methoxycarbonyl-methyl)-benzoic acid (1.75 g) was dissolved in DMF (10 ml). Diisopropylethylamine (5 eq), HOBt (1 eq) and TBTU (1.3 eq) were added. The mixture was stirred at RT for 30 minutes and 4-aminopyridine (1 eq) was added. The mixture was stirred at RT for 2 days. The solvent was removed under reduced pressure. The residue was taken in DCM (100 ml) and extracted with 1N sodium bicarbonate (3×100 ml). The organic layer was dried over MgSO₄, filtered and the solvent was removed under reduced pressure. The residue was purified by flash chromatography (DCM/MeOH:99/1 to 95/5) to yield the tert-butoxycarbonylamino-[4-(pyridin-4-ylcarbamoyl)-phenyl]-acetic acid methyl ester as a yellow powder (56% yield).

Lithium hydroxide (1.2 eq) was added to a solution of tert-butoxycarbonylamino-[4-(pyridin-4-ylcarbamoyl)-phenyl]-acetic acid methyl ester (712 mg) in water/acetone (10 ml): 9/1. The mixture was stirred at RT. After 18 hours 1.2 more equivalent of lithium hydroxide was added. After 24 hours (total reaction time) the reaction was complete. The mixture was neutralized to pH=7 with 1N HCl. The water layer was extracted with ethyl acetate (3×30 ml). The combined organic layers were dried over MgSO₄, filtered and the solvent was removed under reduced pressure to yield the tert-butoxycarbonylamino-[4-(pyridin-4-ylcarbamoyl)-phenyl]-acetic acid as a yellow powder (56% yield). 1H NMR (300 MHz, DMSO-d6): 1.39 ppm (s, 9H); 5.20 ppm (d, 1H, J=8.0 Hz); 7.56 ppm (d, 2H, J=8.3 Hz); 7.77 ppm (d, 2H, J=6.3 Hz); 7.92 ppm (d, 2H, J=8.3 Hz); 8.47 ppm (d, 2H, J=6.3 Hz); 10.60 ppm (s, 1H).

### Intermediate 2: (2-amino-{[4-(pyridin-4-ylcarbamoyl)-phenyl]-ethyl}-carbamic acid tert-butyl ester

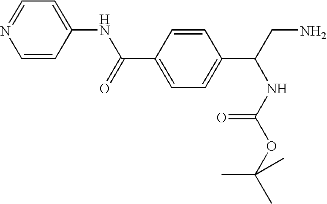

To a solution of 2-amino-1-(4-bromo-phenyl)ethan-1-one hydrochloride (10 g) in dry THF (200 ml), were added DIEA (1 eq) and benzylchloroformate (1.1 eq). The reaction mixture was stirred overnight at RT. The solution was concentrated under reduced pressure. The resulting white solid was separated between DCM (400 ml) and water (175 ml). The organic phase was dried over MgSO₄, filtered and evaporated. The residue was dried to give the [2-(4-Bromo-phenyl)-2-oxo-ethyl]-carbamic acid benzyl ester as a white powder (84% yield).

The [2-(4-Bromo-phenyl)-2-oxo-ethyl]-carbamic acid benzyl ester (6.8 g) was dissolved in THF (52 ml) and water (8 ml). Potassium acetate (1 eq), 1,3-bis-diphenylphosphinopropane (0.02 eq) and Pd(OAc)₂ (0.04 eq) were added. The mixture was stirred under 50 atm of carbon monoxyde at 150° C. for 3 hours. The reaction mixture was cooled down at RT and then filtered. The solvent was evaporated under reduced pressure. The residue was dissolved in EtOAc and extracted with 0.1N HCl. The organic layer was dried over MgSO₄, filtered and the solvent was removed under reduced pressure. The residue was dried to give the 4-(2-benzyloxycarbonylamino-acetyl)-benzoic acid as an orange powder (94% yield).

To a solution of 4-(2-benzyloxycarbonylamino-acetyl)-benzoic acid (2.6 g) in DCM (0.25 M) were added oxalyl chloride (2.5 eq) and a few drops of DMF. The solution was stirred at RT for 2 hours and then evaporated to give the 4-(2-benzyloxycarbonylamino-acetyl)-benzoyl chloride. The 4-aminopyridine (0.78 g, 1 eq) was dissolved in acetonitrile (0.25 M) and DIEA (3 eq) was added. The solution was cooled at 0° C. (in an ice bath). The 4-(2-benzyloxycarbonylamino-acetyl)-benzoyl chloride in the minimum of acetonitrile was then added dropwise (under nitrogen). After addition, the ice bath was removed and the reaction mixture was stirred at RT for 3 hours. The solvent was evaporated and the residue was dissolved in DCM and extracted with 1N NaOH. The organic layer was dried over MgSO₄, filtered and the solvent was removed under reduced pressure. The residue was purified by flash chromatography (DCM/MeOH 97/3 to 95/5) to give the {2-oxo-2-[4-(pyridin-4-ylcarbamoyl)-phenyl]-ethyl}-carbamic acid benzyl ester as a white powder (37% yield).

The {2-oxo-2-[4-(pyridin-4-ylcarbamoyl)-phenyl]-ethyl}-carbamic acid benzyl ester (1.3 g) was dissolved in EtOH (0.25 M). DIEA (5 eq) and hydroxylamine hydrochloride (5 eq) were added. The reaction mixture was stirred at 60° C. for 12 hours and then cooled down at RT. The solvent was concentrated under reduced pressure, and then water was added to the residue. The {2-hydroxyimino-2-[4-(pyridin-4-ylcarbamoyl)-phenyl]-ethyl}-carbamic acid benzyl ester was collected by filtration and dried (yellowish powder, 59% yield).

The oxime was dissolved in acetic acid (0.25 M), and then zinc powder was added (10 eq). The reaction was stirred at RT for 3 hours. Zinc was filtered off and washed with water. The filtrate was evaporated, and the resulting white solid was dissolved in water. The pH was brought to 14 (with NaOH) and the aqueous phase was extracted with EtOAc. The organic layer was dried over MgSO₄, filtered and the solvent was removed under reduced pressure. The residue was dried to give the {2-amino-2-[4-(pyridin-4-ylcarbamoyl)-phenyl]-ethyl}-carbamic acid benzyl ester as a white powder. The amine (1.2 g) was dissolved in acetonitrile (0.25 M) and then, DIEA (3 eq) and (BOC)₂O (1.1 eq) were added. The reaction mixture was stirred at RT for 2 hours and then was evaporated. The residue was dissolved in EtOAc and extracted with 1N NaHCO₃. The organic layer was dried over MgSO₄, filtered and the solvent was removed under reduced pressure. The residue was purified by flash chromatography (cyclohexane/EtOAc, 20/80 10/90 and 0/100) to give the {2-benzyloxycarbonylamino-1-[4-(pyridin-4-ylcarbamoyl)-phenyl]-ethyl}-carbamic acid tert-butyl ester (60% yield).

To a solution of the {2-benzyloxycarbonylamino-1-[4-(pyridin-4-ylcarbamoyl)-phenyl]-ethyl}-carbamic acid tert-butyl ester (0.5 g) in EtOH/water (1/1) were added acetic acid (2 eq) and Pd (10%, 500 mg). The reaction mixture was stirred at RT under hydrogen (2 atm) for 1 hour. The palladium was filtered off. The filtrate was neutralize with 1N NaOH, and then was evaporated. The residue was dissolved in water. The pH was brought to 14 (with NaOH) and the aqueous phase was extracted with EtOAc. The organic layer was dried over MgSO₄, filtered and the solvent was removed under reduced pressure. The residue was dried to give the title compound as a white powder (47% yield).

### Intermediate 3: 3-tert-butoxycarbonylamino-3-[4-(pyridin-4-ylcarbamoyl)-phenyl]-propionic acid

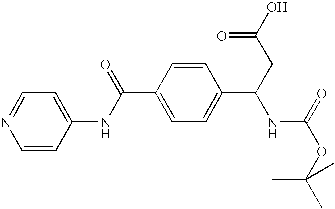

4-(1-Amino-2-carboxy-ethyl)-benzoic acid methyl ester (2.465 g) was suspended in 100 ml of a mixture acetone/1M Na₂CO₃ (9/1). BOC₂O (1.1 eq) was added and the reaction mixture was stirred at RT for 3 hours. Further 2 equivalents of BOC₂O were added and the reaction mixture was stirred for 2 hours. Acetone was removed under reduced pressure. The residue was acidified (pH=2) with 1M HCl. The precipitate was filtered off and washed with water to give the 4-(1-tert-butoxycarbonylamino-2-carboxy-ethyl)-benzoic acid methyl ester as a white powder (88% yield).

4-(1-tert-Butoxycarbonylamino-2-carboxy-ethyl)-benzoic acid methyl ester (2.845 g) was suspended in DMF (60 ml). K₂CO₃ (26 eq), Benzyl-triethyl ammonium chloride (BTEAC; 1 eq) and tert-butyl bromide (48 eq) were added. The reaction mixture was stirred at 55° C. for 5 hours before addition of 10 equivalents tert-butyl bromide. The reaction mixture was stirred for 2 hours further more and then concentrated under reduced pressure. The solution was concentrated and water was added. The solution was extracted with ethyl acetate. The organic layer was washed with 0.05 M NaHCO₃, dried over MgSO₄ and evaporated under reduced pressure. The residue was purified by flash chromatography (cyclohexane/EtOAc: 100/0 to 80/20) to give the 4-(2-tert-butoxycarbonyl-1-tert-butoxycarbonylamino-ethyl)-benzoic acid methyl ester as a yellow powder (67% yield).

To a solution of 4-(2-tert-butoxycarbonyl-1-tert-butoxycarbonylamino-ethyl)-benzoic acid methyl ester (2.13 g) in methanol (0.25 M) was 1M LiOH (5.6 ml). The reaction mixture was stirred at 35° C. for 6 hours. Further 0.5 eq of LiOH was added and the reaction mixture was stirred at RT overnight. The reaction mixture was evaporated. The residue was taken in water. The solution was acidified (pH=2) with 1 M HCl and extracted with ethyl acetate. The organic layers were dried over MgSO₄, and evaporated. The residue was purified by flash chromatography (cyclohexane/ethyl acetate 80/20 to 60/40) to give the 4-(2-tert-butoxycarbonyl-1-tert-butoxycarbonylamino-ethyl)-benzoic acid as a white powder (44% yield).

To a solution of 4-(2-tert-butoxycarbonyl-1-tert-butoxycarbonylamino-ethyl)-benzoic acid in DMF (0.25 M) were added DIEA (5 eq) and a solution of TBTU/HOBt (1 eq/0.2 eq) 0.4M in DMF. After 4 min of stirring, the 4-aminopyridine (1 eq) was added and the reaction mixture was stirred for 1 h before addition of 1 extra equivalent of DIEA and TBTU. After a total of 3 h of stirring, the reaction was completed. The reaction mixture was concentrated under reduced pressure. The residue was taken in ethyl acetate, and washed with 1 M NaHCO₃, and then with brine. The organic layer was dried over MgSO₄, filtered and evaporated. The residue was purified by flash chromatography (AcOEt/cyclohexane 1/4 to 4/1) to give 3-tert-butoxycarbonylamino-3-[4-(pyridin-4-ylcarbamoyl)-phenyl]-propionic acid tert-butyl ester as a pale orange powder (95% yield).

To a solution of 3-tert-butoxycarbonylamino-3-[4-(pyridin-4-ylcarbamoyl)-phenyl]-propionic acid tert-butyl ester (2.25 g) in THF (0.25 M) was added 1M LiOH (3 eq). The reaction mixture was stirred at 30° C. for 20 The solution was concentrated under reduced pressure and then acidified with 1 M HCl (pH=5). The solution was extracted with ethyl acetate (3×100 ml). The combined organic layers were dried over MgSO₄, filtered and evaporated. The residue was purified by flash chromatography (AcOEt/MeOH: 1/0 to 0/1) to give 3-tert-butoxycarbonylamino-3-[4-(pyridin-4-ylcarbamoyl)-phenyl]-propionic acid as a white powder (58% yield). 1H NMR (300 MHz, DMSO-d6): 1.33 ppm (s, 9H); 2.57-2.75 ppm (m, 2H); 4.93 ppm (m, 1H); 7.46 ppm (d, 2H, J=8.3 Hz); 7.52 ppm (d, 1H, J=8.5 Hz); 7.80 ppm (d, 2H, J=6.5 Hz); 7.89 ppm (d, 2H, J=8.2 hz); 8.47 ppm (d, 2H, J=6.5 Hz); 10.62 ppm (s, 1H).

### Intermediate 4: (R)-3-tert-butoxycarbonylamino-3-[4-(pyridin-4-ylcarbamoyl)-phenyl]-propionic acid

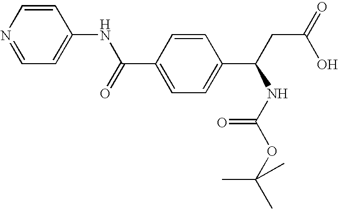

To a solution of (R)-3-(4-bromo-phenyl)-3-tert-butoxycarbonylamino-propionic acid (5.3 g) in DCM (100 ml), were added TBTU (1 eq.) and HOBt (1 eq.). The mixture was cooled at 0° C. and then, DIEA (1.2 eq.) was added dropwise. The reaction mixture was stirred at 0° C. for 15 min. Methanol (20 ml) was then added, and the solution was stirred at RT for 12 hours. Solvent was evaporated, DCM (100 ml) was added and the solution was washed with 1M NaHCO₃ (2×100 ml), 20% KHSO4 (2×100 ml) and brine (2×100 ml). The organic layer was dried over Na₂SO₄ and evaporated, yielding the (R)-3-(4-bromo-phenyl)-3-tert-butoxycarbonylamino-propionic acid methyl ester (99% yield). 1H NMR (300 MHz, DMSO-d6): 1.34 ppm (s, 9H); 2.70 ppm (m, 2H); 3.53 ppm (s, 3H); 4.85 ppm (m, 1H); 7.24 ppm (d, 2H, J=8.4 Hz); 7.49 ppm (m, 3H).

To a solution of (R)-3-(4-bromo-phenyl)-3-tert-butoxycarbonylamino-propionic acid methyl ester (5.2 g) in a mixture of THF (65 ml) and water (10 ml) were successively added potassium acetate (1 eq.), palladium acetate (0.04 eq.) and DPPP (0.02 eq.). The reaction mixture was stirred at 150° C. under 50 atm of carbon monoxide, for 3 hours. The reaction mixture was cooled down at RT, and then was filtered off. The filtrate was evaporated. The residue was purified by flash chromatography on silica gel (DCM/MeOH, 100/0 to 95/5). The 4-((R)-1-tert-butoxycarbonylamino-2-methoxycarbonyl-ethyl)-benzoic acid was obtained as a white powder (53% yield).

To a solution of 4-((R)-1-tert-butoxycarbonylamino-2-methoxycarbonyl-ethyl)-benzoic acid (2.4 g) in DMF (25 ml) were added TBTU (1.3 eq.), HOBt (0.2 eq.) and DIEA (3 eq.). The reaction mixture was stirred at RT for 5 min, and the 4-aminopyridine (1 eq.) was added. The solution was stirred at RT for 3 hours. DMF was evaporated, and water was added to the residue. The product was extracted with EtOAc (150 ml). The organic layer was washed with brine (100 ml), dried over MgSO₄ and evaporated. The residue was purified by flash chromatography on silica gel (EtOAc), yielding the (R)-3-tert-butoxycarbonylamino-3-[4-(pyridin-4-ylcarbamoyl)-phenyl]-propionic acid methyl ester as a white powder (67% yield). 1H NMR (300 MHz, DMSO-d6): 1.34 ppm (s, 9H); 2.75 ppm (m, 2H); 3.55 ppm (s, 3H); 4.97 ppm (m, 1H); 7.46 ppm (d, 2H, J=8.3 Hz); 7.58 ppm (d, 1H, J=8.7 Hz); 7.76 ppm (d, 2H, J=5.1 Hz); 7.88 ppm (d, 2H, J=8.2 Hz); 8.46 ppm (d, 2H, J=5.1 Hz); 10.55 ppm (s, 1H).

To a suspension of (R)-3-tert-butoxycarbonylamino-3-[4-(pyridin-4-ylcarbamoyl)-phenyl]-propionic acid methyl ester (1.9 g) in 1,4-dioxane (35 ml) was added 1N LiOH (1.1 eq. of LiOH). The reaction mixture was stirred at RT for 2 hours and at 4° C. overnight. The pH was adjusted to 7 by addition of 1N HCl (4.5 ml). The solution was lyophilized without further workup (the title product was in mixture with salts which could be removed in the next step). The title compound was obtained as a white powder (yield not determined). 1H NMR (300 MHz, DMSO-d6): 1.34 ppm (s, 9H); 2.62 ppm (m, 2H); 4.91 ppm (m, 1H); 7.45 ppm (d, 2H, J=8.3 Hz); 7.58 ppm (d, 1H, J=9.7 Hz); 7.76 ppm (d, 2H, J=6.4 Hz); 7.88 ppm (d, 2H, J=8.2 Hz); 8.45 ppm (d, 2H, J=6.4 Hz); 10.55 ppm (s, 1H).

### Intermediate 5: (S)-3-tert-butoxycarbonylamino-3-[4-(pyridin-4-ylcarbamoyl)-phenyl]-propionic acid

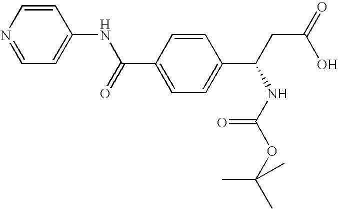

The title compound was prepared according to the protocol described for Intermediate 4 starting from the (S)-3-(4-bromo-phenyl)-3-tert-butoxycarbonylamino-propionic acid. 1H NMR (300 MHz, DMSO-d6): 1.34 ppm (s, 9H); 2.62 ppm (m, 2H); 4.91 ppm (m, 1H); 7.45 ppm (d, 2H, J=8.3 Hz); 7.58 ppm (d, 1H, J=9.7 Hz); 7.76 ppm (d, 2H, J=6.4 Hz); 7.88 ppm (d, 2H, J=8.2 Hz); 8.45 ppm (d, 2H, J=6.4 Hz); 10.55 ppm (s, 1H).

### Intermediate 6: {[4-(Pyridin-4-ylcarbamoyl)-phenyl]-pyrrolidin-2-yl-methyl}-carbamic acid tert-butyl ester

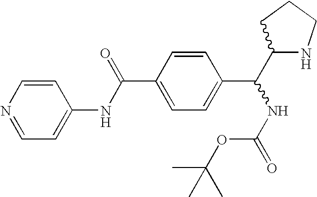

To a solution of Z-L-proline (10 g, 1.2 eq.) in DCM/DMF (15/1 ml), was added oxalyl chloride (1.8 eq.). The reaction mixture was stirred at RT for 3 hours, then the solvent was evaporated to give the Z-L-proline acyl chloride, which was used without further purification.

A solution of methyl 4-iodobenzoate (1 eq.) in dry THF (40 ml) was cooled down to −78° C. Isopropyl magnesium chloride was added dropwise followed by addition of THF (15 ml) the whole precipitate was dissolved, and the reaction mixture was stirred at −78° C. for 30 min. A solution of CuCN.2LiCl in dry THF (1 molar, 33.6 ml) was added and the reaction mixture was stirred for 15 min more. A solution of freshly prepared Z-L-proline acyl chloride in THF (40 ml) was added dropwise, the reaction mixture stirred at −78° C. for 5 min and then was stirred for 55 min, while allowing to reach RT Saturated solution of NH₄Cl was added. The reaction mixture was then extracted with ethyl acetate (×3). The combined organic layers were washed with ammonia solution and then with water, dried over MgSO₄, filtered, and evaporated. The residue was purified by flash chromatography on silica gel (DCM/pentane 1/1 to 2/3) to give the 2-(4-methoxycarbonyl-benzoyl)-pyrrolidine-1-carboxylic acid benzyl ester as an oil (57% yield).

To a solution of the ester in MeOH (0.25 M) was added 1M LiOH (1.1 eq.). The reaction mixture was stirred at RT for 3.5 hours. The solvent was evaporated and the reaction mixture was acidified with 1M HCl and then extracted with ethyl acetate (×3). The combined organic layers were washed with water, dried over MgSO₄, filtered, and evaporated to afford the 2-(4-carboxy-benzoyl)-pyrrolidine-1-carboxylic acid benzyl ester as a white powder (80% yield).

To a solution of the acid in DMF (0.25), were successively added TBTU (1.3 eq.), HOBt (0.3 eq.) and DIEA (3 eq.). The reaction mixture was stirred for 5 min and then, 4-aminopyridine (1 eq.) was added and the reaction mixture stirred at RT for 1 h. The solvent was evaporated and the residue was poured into water and extracted with ethyl acetate (×3). The combined organic layers were washed with NaHCO₃ solution and then with water, dried over MgSO₄, filtered and evaporated to give the 2-[4-(Pyridin-4-ylcarbamoyl)-benzoyl]-pyrrolidine-1-carboxylic acid benzyl ester as a yellow oil (88% yield).

To a solution of the ketone in EtOH (0.25 M) was added hydroxylamine hydrochloride (2 eq.) followed by addition of DIEA (3 eq.). The reaction mixture was heated at 80° C. for 24 hours. The reaction mixture was cooled to RT and the solvent was evaporated. The residue was purified by flash chromatography on silica gel (DCM/MeOH 99/1 to 95/5) to afford the 2-{hydroxyimino-[4-(pyridin-4-ylcarbamoyl)-phenyl]-methyl}-pyrrolidine-1-carboxylic acid benzyl ester as a pale yellow foam (82% yield).

To a solution of the oxime (1 eq.) in acetic acid (0.25 M), was added zinc powder (8.5 eq.) and the reaction mixture was stirred at RT for 3 hours. The reaction mixture was filtered. Zinc was washed with AcOH, and the filtrate was evaporated. The residue was acidified with 1M HCl and washed with ethyl acetate. The aqueous layer was basified with 2M NaOH and extracted with ethyl acetate (×3). The combined organic layers were washed with water, dried over MgSO₄, filtered, and evaporated to give the 2-{amino-[4-(pyridin-4-ylcarbamoyl)-phenyl]-methyl}-pyrrolidine-1-carboxylic acid benzyl ester as a white foam (29% yield), which was used without further purification.

To a solution of the amine in DCM was added di-tert-butyl dicarbonate in DCM dropwise at RT with vigorous stirring. After the starting material disappeared (15 min), water was added and the reaction mixture was stirred for another 10 min. The organic layer was separated, then washed with water, dried over MgSO₄ and finally evaporated. The residue was purified by flash chromatography on silica gel (DCM/MeOH 99/1 to 95/5) to afford the 2-{tert-butoxycarbonylamino-[4-(pyridin-4-ylcarbamoyl)-phenyl]-methyl}-pyrrolidine-1-carboxylic acid benzyl ester as a white powder (64% yield).

To a solution of 2-{tert-butoxycarbonylamino-[4-(pyridin-4-ylcarbamoyl)-phenyl]-methyl}-pyrrolidine-1-carboxylic acid benzyl ester in MeOH was added Pd/C and the reaction mixture was stirred at RT under 1 atm of hydrogen for 5 hours. The reaction mixture was filtered through a layer of Celite and thoroughly washed with MeOH. The solvent was evaporated to give the title compound as a white powder (80% yield).

### Intermediate 7: {2-Methylamino-1-[4-(pyridin-4-ylcarbamoyl)-phenyl]-ethyl}-carbamic acid tert-butyl ester

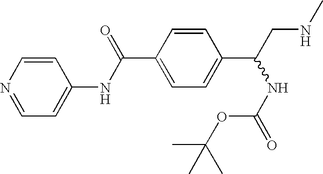

To a solution of 4-acetyl-benzoic acid methyl ester (345.7 g, 1.94 mmol, 1 eq.) in chloroform (1700 ml) was added dropwise bromine (100 ml, 310 g, 1.94 mmol, 1 eq.) in chloroform (3100 ml) with stirring at RT. During addition of bromine the reaction displayed an exotherm of 10° C. After 2 h at RT, the mixture was diluted with ice water (1000 ml) and aqueous Na₂S₂O₃ (700 ml) and extracted with DCM (3×1200 ml). The organic layer was washed with water (4500 ml), dried over MgSO₄ and concentrated in vacuo to give the 4-(2-Bromo-acetyl)-benzoic acid methyl ester (527.2 g). The crude residue was recrystallized from methanol (2500 ml) to give 334 g (67% yield).

To a stirred solution of bromoketone (590.5 g) in MeOH (5900 ml) at 0° C. was added NaBH₄ (91.2 g) portionwise. The reaction was allowed to warm to RT and stirred for 1 h after which time TLC analysis indicated the formation of the bromo alcohol. K₂CO₃ (318 g) was added to the same flask and the reaction mixture stirred over the weekend. TLC analysis indicated the reaction was complete. Water (3000 ml) was added and the mixture extracted with Et₂O (3×5000 ml). The organic extracts were washed with brine (2×5000 ml), dried over MgSO₄ and concentrated in vacuo to give the 4-oxiranyl-benzoic acid methyl ester as an orange solid, 405.8 g (99% yield).

The 4-oxiranyl-benzoic acid methyl ester (405 g) was dissolved in methylamine 33 wt % in EtOH and stirred overnight. TLC analysis indicated the reaction was complete. Water was added and the mixture extracted with EtOAc (4×500 ml). The organic extracts were washed with water (3×500 ml), dried over MgSO₄ and concentrated in vacuo to give 495 g of 4-(1-hydroxy-2-methylamino-ethyl)-benzoic acid methyl ester.

The amino alcohol (412.3 g) was dissolved in THF (6000 ml) and NaHCO₃ (336 g, 2 eq.) was added with stirring. The solution was cooled to 0-5° C. and benzyl chloroformate (416 ml, 1.5 equiv.) in THF (6000 ml) was added dropwise. The mixture was stirred at 0-5° C. for 1 h and allowed to warm to RT overnight. TLC analysis indicated the reaction was complete. Water (9000 ml) was added and the aqueous layer extracted with EtOAc (2×5000 ml). The organic layer was back extracted with saturated aqueous NaHCO₃ solution (2×2500 ml). The combined organic layers were dried over MgSO₄ and concentrated in vacuo to give a crude product, 760.7 g. The crude product was purified by column chromatography to give 4-[2-(Benzyloxycarbonyl-methyl-amino)-1-hydroxy-ethyl]-benzoic acid methyl ester (137 g, 20% yield from the bromoketone).

To a solution of the previous alcohol (137 g, 0.4 mol) in DCM (1400 ml) was added triethylamine (123 ml, 0.88 mol, 2.2 eq.) and the reaction cooled to <5° C. Mesylate chloride (48 ml, 0.6 mol, 1.5 eq.) was added dropwise and after complete addition, the reaction mixture was allowed to warm to RT. After 1 h LC analysis indicated the reaction was complete. The DCM layer was washed with H₂O (1400 ml), 1M HCl (1400 ml) and H₂O (1400 ml). The DCM layer was dried over MgSO₄ and concentrated in vacuo to give the 4-[2-(benzyloxycarbonyl-methyl-amino)-1-methanesulfonyloxy-ethyl]-benzoic acid methyl ester (166.7 g, 99% yield).

To a 2000 ml flask was added the previous mesylated product (166.7 g, 0.4 mol) and DMF (1700 ml). NaN₃ (25.7 g, 0.4 mol, 1 eq.) was added portionwise. The reaction mixture was heated to 50° C. and stirred for 14 h. LC analysis indicated the reaction was complete. The reaction was cooled to RT and Ph₃P (105 g, 0.4 mol, 1 eq.) and H₂O (105 ml) were added. The reaction was stirred for 2 h and LC analysis indicated the reaction was complete. The reaction mixture was concentrated in vacuo to give the 4-[1-amino-2-(benzyloxycarbonyl-methyl-amino)-ethyl]-benzoic acid methyl ester as a sticky solid (351.8 g), which was used without further purification.

The amine (351.8 g, active charge 135 g, 0.39 mol) was dissolved in a mixture of 1:1 acetone/1M Na₂CO₃ solution (5000 ml). Boc anhydride (197 ml, 0.86 mol, 2.2 eq.) was added and the reaction mixture stirred overnight at RT. LC analysis indicated the reaction was complete. The acetone was removed in vacuo and the aqueous layer was extracted with EtOAc (3×2000 ml). The combined organic extracts were washed with brine (3000 ml), dried over MgSO₄ and concentrated in vacuo. The 4-[2-(benzyloxycarbonyl-methyl-amino)-1-tert-butoxycarbonylamino-ethyl]-benzoic acid methyl ester was purified by flash chromatography on silica gel (21% yield from the CBz protected amino alcohol).

To a solution of the previous ester (35.9 g) in MeOH (1500 ml) was added 1M NaOH solution (700 ml) slowly. The reaction mixture was stirred for 4 h after which time the reaction was complete. The methanol was removed in vacuo, the aqueous layer acidified to pH 5-6 using 0.5M HCl (1400 ml) and the product extracted with EtOAc (3×1500 ml). The organic layer was dried over MgSO₄, filtered and concentrated in vacuo to give the corresponding benzoic acid (100% yield).

To a solution of the acid (34.76 g, 0.081 mol) in DMF (1000 ml) were added DIEA (42 ml, 0.243 mol, 3 equiv.), HBTU (40 g, 0.1053 mol, 1.3 equiv.), HOBt (3.2 g, 0.0243 mol, 0.3 equiv.) and 4-aminopyridine (9.15 g, 0.0972 mol, 1.2 equiv.). The reaction mixture was stirred overnight at RT. TLC analysis indicated the reaction was complete. DMF was evaporated and the residue taken up in EtOAc (2500 ml) and 1M Na₂CO₃ (2500 ml). The layers were separated and the aqueous layer was extracted with EtOAc (2500 ml). The organic layer was washed with brine (5000 ml), dried over MgSO₄ and concentrated in vacuo. The {2-(benzyloxycarbonyl-methyl-amino)-1-[4-(pyridin-4-ylcarbamoyl)-phenyl]-ethyl}-carbamic acid tert-butyl ester was purified by flash chromatography on silica gel (85% yield).

The previous compound (34.7 g) was dissolved in MeOH (250 ml) and transferred to a 300 ml Parr hydrogenator vessel. The vessel was purged with N₂ and 10% Pd/C (wet catalyst) (20 g) were added. The reaction was purged with hydrogen and stirred for 5 h at 5 bar pressure of hydrogen. TLC analysis indicated the reaction was complete. The reaction mixture was filtered through Celite (100 g) and the filter cake washed with MeOH (750 ml). The reaction mixture was concentrated in vacuo. The crude residue was purified by flash chromatography on silica gel to give the title compound (67% yield).

### General Procedures:

### Protocol A:

To a solution of the corresponding carboxylic acid (5.25 μmol) in DMF (0.437 M) with DIEA (3 eq) was added 1 eq of a solution of TBTU/HOBt (1/0.2) in DMF (0.4 M). The reaction mixture was stirred at RT for 3 to 10 minutes and a solution of the corresponding amine (1 eq) in DMF (0.1 M) at neutrality (with DIEA). The reaction mixture was stirred at RT for 3 hours, and then 0.7 eq of a solution of TBTU/HOBt (1/0.2) in DMF (0.4 M) was added. After 4 hours the reaction mixture was evaporated.

Deprotection of tert-butoxycarbonylamino group: a mixture of DCM and trifluoroacetic acid (1/1; 100 μl) was added to the residue. The solution was stirred at RT for 2 hours, and then evaporated under reduced pressure. Compounds were used without further purification.

### Protocol B

To a solution of the corresponding amine (5.25 μmol) in DMF (0.1 M) at neutrality (with DIEA if needed) was added a solution of the corresponding sulfonyl chloride (1 eq) in dry THF (0.25 M). The reaction mixture was stirred at RT for 2 to 8 hours, and then evaporated.

Deprotection of tert-butoxycarbonylamino group: a mixture of DCM and trifluoroacetic acid (1/1; 100 μl) was added to the residue. The solution was stirred at RT for 2 hours, and then evaporated under reduced pressure. Compounds were used without further purification.

### Protocol C:

To a solution of the corresponding carboxylic acid (1 eq) in DMF (0.25 M) with DIEA (3 eq) was added TBTU (1.3 eq) and HOBt (0.3 eq). The reaction mixture was stirred at RT for 3 to 10 minutes and the corresponding amine (1 eq) was added. The reaction mixture was stirred at RT for 3 hours to 3 days. The solvent was evaporated. The residue was partitioned between EtOAc and 2N Na₂CO₃ (or 1N NaOH). The product was extracted with EtOAc. The organic layer was separated, washed with brine, dried over magnesium sulfate, and evaporated.

Alternative protocol: To a solution of the corresponding carboxylic acid (1 eq) in a mixture DMF/DCM (0.25 M) were successively added DCC (1 eq), HOBt (1 eq) and DIEA (3 eq). The solution was stirred at RT for 30 minutes before the addition of the corresponding amine (1 eq). The reaction mixture was stirred at RT for 2 hours to 3 days. The solvent was evaporated. The residue was partitioned between DCM and water. The product was extracted with DCM. The organic layer was separated, washed with 2N Na₂CO₃ (or 1N NaOH), brine, dried over magnesium sulfate, and evaporated.

Deprotection of tert-butoxycarbonylamino group: The crude product was dissolved in freshly distillated 1,4-dioxane. HCl gas was bubbled in the solution for 10 to 30 minutes. The solvent was evaporated and then, the residue was purified by preparative HPLC.

### Protocol D

Compound 157 to 168 can be made following the general scheme hereafter (synthesis of Compound 166 as an example):

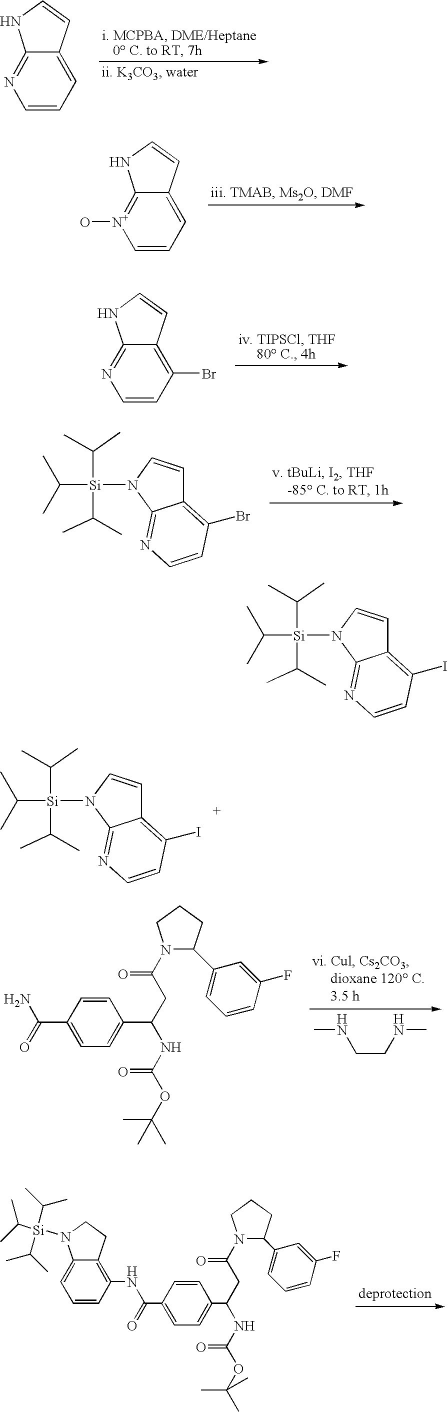

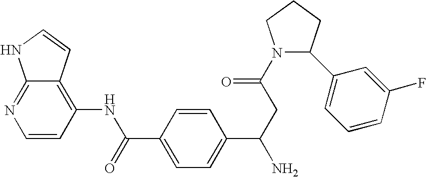

### Biological Activity

The compounds were tested for inhibition of human ROCKα/ROCKII mix.

The inhibition assays were performed with a fluorescence polarization (FP) assay using the commercially available ROCK IMAP Kit from Molecular Devices (Product ID. No. R8093), essentially in accordance with the protocol supplied by the manufacturer. The S6 ribosomal protein-derived substrate used was (FI)-AKRRRLSSLRA, also obtained from Molecular Devices (Product ID No. R7184). The enzyme mix ROCKα/ROCKII was obtained from Upstate Biotechnology (Product ID No 14-451).

In summary, all compounds were screened in the wells of a 384 well plate for enzymatic inhibition with concentrations varying from 100 μM to 0.3 nM using a stepwise 3 (or 2)-fold dilution. Y compound (Y-27632 commercially available from Tocris) was used as a reference (0.4 μM).

To perform the assay, 1 μl of a solution of the compound to be tested in DMSO (at each concentration) was added to 2 μl of a solution of the enzyme in 10 mM Tris-HCl, 10 mM MgCl₂, 0.1% BSA, 0.05% NaN₃, pH 7.2. The final concentration of the enzyme was 2.6 nM.

After incubating for 30 minutes at RT, 2 μl of a mixture of ATP and the protein substrate in 10 mM Tris-HCl, 10 mM MgCl₂, 0.1% BSA, 0.05% NaN₃, pH 7.2 was added. The final concentration of the ATP was 10 μM and final concentration of protein substrate was 0.2 μM.

After incubating for 60 minutes at RT, 12 μl of the IMAP Binding Solution (mix of the IMAP Binding Buffer A (1×) and the IMAP Binding Reagent (from the ROCK IMAP kit)) was added.

The mixture thus obtained (total volume: 17 μl) was incubated for 60 minutes at RT, upon which the fluorescence polarization was measured using an automated plate reader (Perkin Elmer, Model Envision 2100-0010 HTS) with FP filters: excitation filter FITC FP 480 and emission filters FITC FP P-pol 535 and FITC FP S-pol 535 (Perkin-Elmer). The results were fitted to a curve using the XL-Fit algorithm and IC50 values were calculated for each fitted curve, again using the XL-Fit algorithm.

The IC₅₀ value for the reference compound (Y compound Y-27632) was 0.4 μM.

### Compounds of the Invention

In the tables 1, 2, 3, 4, 5, 6 and 7 that are set forth below, exemplary compounds of the invention are set out in tabulated form. In these tables, the name of the compound, an arbitrarily assigned compound number and structural information are set out. In addition, the protocol by which the compounds were made is provided and the IC₅₀ value obtained (in accordance with the protocol set forth above) is represented as follows: “+++” means IC₅₀ below 0.5 μM; “++” means IC₅₀ between 0.5 and 5 μM; “+” means IC₅₀ between 5 and 50 μM, and “nd” means “not determined yet”.

### Attribution of the Configuration:

The Cahn-Ingold-Prelog system was used to attribute the absolute configuration of chiral center, in which the four groups on an asymmetric carbon are ranked to a set of sequences rules. Reference is made to Cahn; Ingold; Prelog Angew. Chem. Int. Ed. Engl. 1966, 5, 385-415.

In the configuration R,R for example the first letter refers to the configuration of the carbon bearing the amine group.

### Name of the Molecule

The software MDL ISIS™/Draw 2.5 was used to assign the name of the molecules.

### Extra Analytical (or Preparative) Techniques:

Unless indicated otherwise, purification by preparative HPLC was performed on a Shimadzu SCL-10A (UV detection at 215 and 254 nm, detector SPD-10A) using C-18 column (Nucleosil, 100 Å, 100 μm, 20×200 mm) and different gradients (water, acetonitrile, formic acid).

Chiral HPLC (analytical and preparative) was performed on a Shimadzu SCL-10A (UV detection at 215 and 254 nm, detector SPD-10A) using different column such as Chiralcel OD-H (tris-3,5-dimethylphenylcarbamate, 46×250 or 100×250 mm, 5 μm), Chiralcel OJ (tris-methylbenzoate, 46×250 or 100×250 mm, 5 μm), Chiralpak AD (tris-3,5-dimethylphenylcarbamate, 46×250 mm, 10 μm) and Chiralpak AS (tris-(S)-1-phenylethylcarbamate, 46×250 mm, 10 μm) from Chiral Technologies Europe (Illkirch, France):
Eluent: mixture of solvent such as ethanol, 1-propanol, 2-propanol, methanol, butanol, pentane, hexane, heptane, cyclohexane, diisopropylethyamine, triethylamine.
Flow: between 1 and 50 ml/min.

Table 1 shows the results for compounds of Formula IV20 wherein R⁸ is H. Table 2 shows the results for compounds of Formula V20 wherein R⁸ is H. Table 3 shows the results for compounds of Formula III20 wherein R⁸ is H. Table 4 shows the results for compounds 132 to 156. Table 5 shows the results for compounds of Formula IV2. Table 6 shows the results for compounds of Formula V2. Table 7 shows the results for compounds of Formula III21. As used herein the term “ND” means “not determined yet” and “Pr” is “Protocol”.

TABLE 1

| | |
|---|---|
| | IV20 |
| 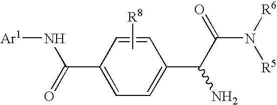 | |

| | | | | | | IC₅₀ |
|---|---|---|---|---|---|---|
| | | | | | | μM |
| Name | Compound | Ar¹— | —R⁵ | —R⁶ | Pr | ROCK |
| 4-(amino- benzylcarbamoyl)- methyl)-N-pyridin-4-yl- benzamide | Compound 1 | 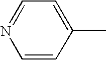 | —H | 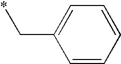 | A | +++ |
| 4-[amino-(1-phenyl- ethylcarbamoyl)- methyl]-N-pyridin-4-yl- benzamide | Compound 2 | 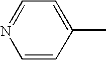 | —H | 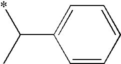 | A | +++ |
| 4-[amino-(benzyl- methyl-carbamoyl)- methyl]-N-pyridin-4-yl- benzamide | Compound 3 | 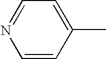 | —CH₃ | 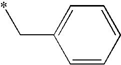 | A | +++ |
| 4-[amino-(1-phenyl- propylcarbamoyl)- methyl]-N-pyridin-4-yl- benzamide | Compound 4 | 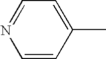 | —H | 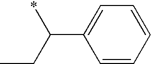 | A | +++ |
| 4-[amino-(1-(S)-phenyl- propylcarbamoyl)- methyl]-N-pyridin-4-yl- benzamide | Compound 5 | 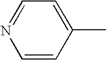 | —H | 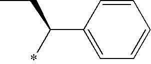 | A | ++ |
| 4-[amino-(2-chloro- benzylcarbamoyl)- methyl]-N-pyridin-4-yl- benzamide | Compound 6 | 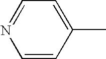 | —H | 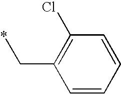 | A | +++ |
| 4-[amino-(3-chloro- benzylcarbamoyl)- methyl]-N-pyridin-4-yl- benzamide | Compound 7 | 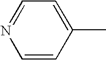 | —H | 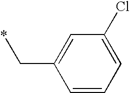 | A | +++ |
| 4-[amino-(4-methoxy- benzylcarbamoyl)- methyl]-N-pyridin-4-yl- benzamide | Compound 8 | 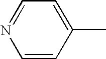 | —H | 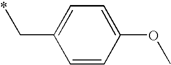 | A | +++ |
| 4-(amino-(2-methoxy- benzylcarbamoyl)- methyl]-N-pyridin-4-yl- benzamide | Compound 9 | 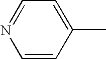 | —H | 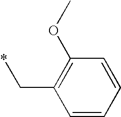 | A | +++ |
| 4-{amino-[(pyridin-2- ylmethyl)-carbamoyl]- methyl}-N-pyridin-4-yl- benzamide | Compound 10 | 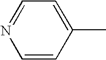 | —H | 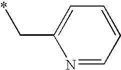 | A | ++ |
| 4-{amino-[(pyridin-4- ylmethyl)-carbamoyl]- methyl}-N-pyridin-4-yl- benzamide | Compound 11 | 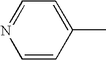 | —H | 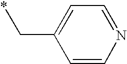 | A | ++ |
| 4-{amino-[(furan-2- ylmethyl)-carbamoyl]- methyl}-N-pyridin-4-yl- benzamide | Compound 12 | 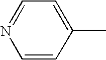 | —H | 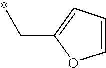 | A | +++ |
| 4-[1-amino-2-(3,4- dihydro-1H-isoquinolin- 2-yl)-2-oxo-ethyl]-N- pyridin-4-yl-benzamide | Compound 13 | 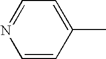 | 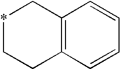 | | A | +++ |
| 4-(amino- phenethylcarbamoyl- methyl)-N-pyridin-4-yl- benzamide | Compound 14 | 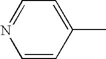 | —H | 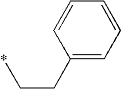 | A | +++ |
| 4-[amino-(indan-2- ylcarbamoyl)-methyl]-N- pyridin-4-yl-benzamide | Compound 15 | 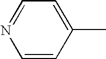 | —H | 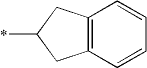 | A | +++ |
| 4-{amino-[2-(4-fluoro- phenyl)ethylcarbamoyl]- methyl}-N-pyridin-4-yl- benzamide | Compound 16 | 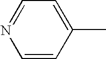 | —H | 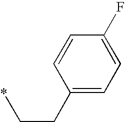 | A | +++ |
| 4-[amino-(2-thiophen-2- yl-ethylcarbamoyl)- methyl]-N-pyridin-4-yl- benzamide | Compound 17 | 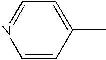 | —H | 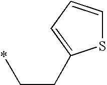 | A | +++ |
| 4-[amino-(2-pyrazol-1- yl-ethylcarbamoyl)- methyl]-N-pyridin-4-yl- benzamide | Compound 18 | 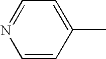 | —H | 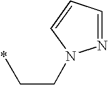 | A | ++ |
| 4-[amino-(2-imidazol-1- yl-ethylcarbamoyl)- methyl]-N-pyridin-4-yl- benzamide | Compound 19 | 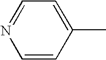 | —H | 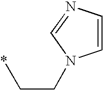 | A | ++ |
| 4-(amino- phenylcarbamoyl- methyl)-N-pyridin-4-yl- benzamide | Compound 20 | 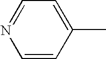 | —H | 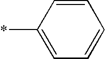 | A | ++ |
| 4-[amino-4-methoxy- phenylcarbamoyl)- methyl]-N-pyridin-4-yl- benzamide | Compound 21 | 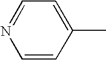 | —H | 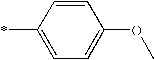 | A | +++ |
| 4-(amino- ethylcarbamoyl-methyl)- N-pyridin-4-yl- benzamide | Compound 22 | 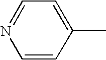 | —H | 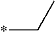 | A | ++ |
| 4-[amino-(1,2-dimethyl- propylcarbamoyl)- methyl]-N-pyridin-4-yl- benzamide | Compound 23 | 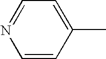 | —H | 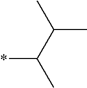 | A | ++ |
| 4-[amino-(1,1-dimethyl- propylcarbamoyl)- methyl]-N-pyridin-4-yl- benzamide | Compound 24 | 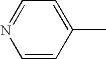 | —H | 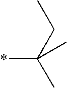 | A | ++ |
| 4-[amino- (cyclopropylmethyl- carbamoyl)-methyl]-N- pyridin-4-yl-benzamide | Compound 25 | 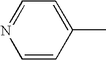 | —H | 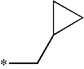 | A | ++ |
| 4-[1-amino-2-(1-methyl- piperidin-1-yl)-2-oxo- ethyl]-N-pyridin-4-yl- benzamide | Compound 26 | 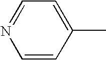 | 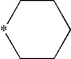 | | A | +++ |
| 4-[1-amino-2-(3,5- dimethyl-piperidin-1-yl)- 2-oxo-ethyl]-N-pyridin-4- yl-benzamide | Compound 27 | 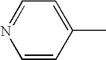 | 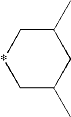 | | A | +++ |
| 4-[1-amino-2-(4-phenyl- piperidin-1-yl)-2-oxo- ethyl]-N-pyridin-4-yl- benzamide | Compound 28 | 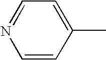 | 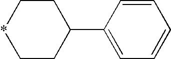 | | A | ++ |
| 4-{1-amino-2-[4-(4- fluoro-phenyl)-3,6- dihydro-2H-pyridin-1-yl]- 2-oxo-ethyl}-N-pyridin- 4-yl-benzamide | Compound 29 | 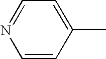 | 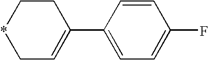 | | A | ++ |
| 4-[1-amino-2-(4- benzooxazol-2-yl- piperidin-1-yl)-2-oxo- ethyl]-N-pyridin-4-yl- benzamide | Compound 30 | 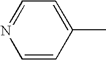 | 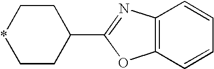 | | A | +++ |
| 4-[1-amino-2-(4-benzyl- piperidin-l-yl)-2-oxo- ethyl]-N-pyridin-4-yl- benzamide | Compound 31 | 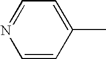 | 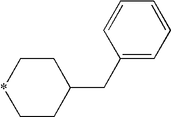 | | A | ++ |
| 4-(amino- cyclohexylcarbamoyl- methyl)-N-pyridin-4-yl- benzamide | Compound 32 | 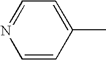 | —H | 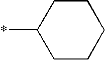 | A | ++ |
| 4-[amino-(4-methyl- cyclohexylcarbamoyl)- methyl]-N-pyridin-4-yl- benzamide | Compound 33 | 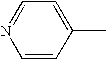 | —H | 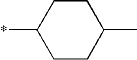 | A | ++ |
| 4-(1-amino-2-mopholin- 4-yl-2-oxo-ethyl)-N- pyridin-4-yl-benzamide | Compound 34 | 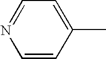 | 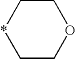 | | A | +++ |
| 4-[1-amino-2-(2,6- dimethyl-mopholin-4-yl)- 2-oxo-ethyl]-N-pyridin-4- yl-benzamide | Compound 35 | 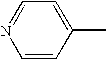 | 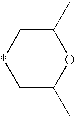 | | A | ++ |
| 4-[1-amino-2-(3- hydroxymethyl- piperidin-1-yl)-2-oxo- ethyl]-N-pyridin-4-yl- benzamide | Compound 36 | 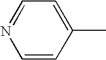 | 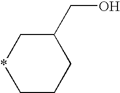 | | A | ++ |
| 4-{amino-[(tetrahydro- furan-2-ylmethyl)- carbamoyl]-methyl}-N- pyridin-4-yl-benzamide | Compound 37 | 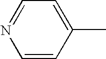 | 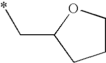 | | A | ++ |
| 4-[1-amino-2-(4- hydroxy-4-phenyl- piperidin-1-yl)-2-oxo- ethyl]-N-pyridin-4-yl- benzamide | Compound 38 | 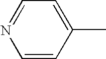 | 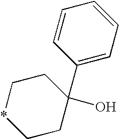 | | A | ++ |
| 4-[amino-(2-methoxy- ethylcarbamoyl)- methyl]-N-pyridin-4-yl- benzamide | Compound 39 | 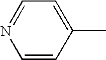 | —H | 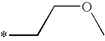 | A | ++ |
| 4-[amino-(2- methanesulfonylamino- ethylcarbamoyl)- methyl]-N-pyridin-4-yl- benzamide | Compound 40 | 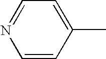 | —H | 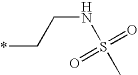 | A | ++ |
| 4-(amino- methoxycarbamoyl- methyl)-N-pyridin-4-yl- benzamide | Compound 41 | 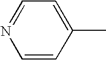 | —H | 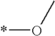 | A | ++ |
| 4-{amino-[(2-hydroxy- ethyl)-methyl- carbamoyl]-methyl}-N- pyridin-4-yl-benzamide | Compound 42 | 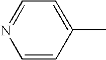 | —CH₃ | 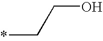 | A | ++ |
| 4-[amino-(2-methoxy-1- methyl-ethylcarbamoyl)- methyl]-N-pyridin-4-yl- benzamide | Compound 43 | 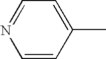 | —H | 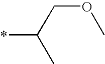 | A | ++ |
| 4-[amino-2- dimethylamino- ethylcarbamoyl)- methyl]-N-pyridin-4-yl- benzamide | Compound 44 | 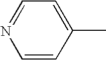 | —H | 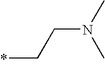 | A | ++ |
| 4-[amino-(2-morpholin- 4-yl-ethylcarbamoyl)- methyl]-N-pyridin-4-yl- benzamide | Compound 45 | 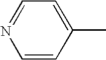 | —H | 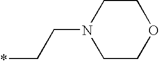 | A | ++ |
| 4-[1-amino-2-(4- isopropyl-piperazin-1- yl)-2-oxo-ethyl]-N- pyridin-4-yl-benzamide | Compound 46 | 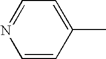 | 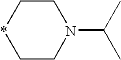 | | A | ++ |
| 4-[1-amino-2-oxo-2-(4- phenyl-piperzin-1-yl)- ethyl]-N-pyridin-4-yl- benzamide | Compound 47 | 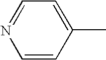 | 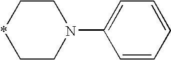 | | A | ++ |
| 4-{1-amino-2-[4-(2- fluoro-benzyl)-piperazin- 1-yl]-2-oxo-ethyl}-N- pyridin-4-yl-benzamide | Compound 48 | 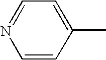 | 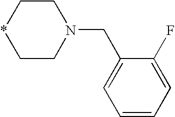 | | A | ++ |
| 4-{1-amino-2-[4-(4- fluoro-benzyl)-piperazin- 1-yl]-2-oxo-ethyl}-N- pyridin-4-yl-benzamide | Compound 49 | 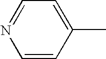 | 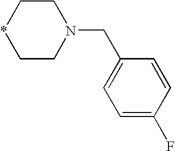 | | A | ++ |
| 4-{1-amino-2-[4-(4- methoxy-benzyl)- piperazin-1-yl]-2-oxo- ethyl}-N-pyridin-4-yl- benzamide | Compound 50 | 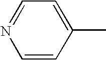 | 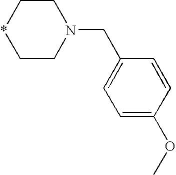 | | A | ++ |
| 4-{amino-[1-benzyl- piperidin-4- ylcarbamoyl]-methyl}-N- pyridin-4-yl-benzamide | Compound 51 | 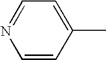 | —H | 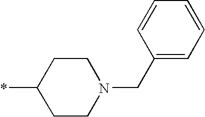 | A | ++ |
| 4-{1-amino-2-[4-(2- hydroxy-ethyl)- piperazin-1-yl]-2-oxo- ethyl}-N-pyridin-4-yl- benzamide | Compound 52 | 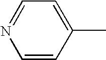 | 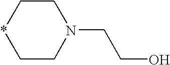 | | A | ++ |
| 4-{amino-[1-(5-methyl- thiophen-2-ylmethyl)- piperidin-4- ylcarbamoyl]-methyl}-N- pyridin-4-yl-benzamide | Compound 53 | 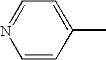 | —H | 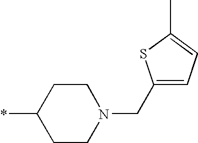 | A | ++ |
| 4-[amino-(N′-butyl- hydrazinocarbonyl)- methyl]-N-pyridin-4-yl- benzamide | Compound 54 | 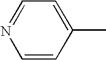 | —H | 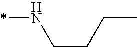 | A | +++ |
| 4-{1-amino-2-[2-(3- fluoro-phenyl)- pyrrolidin-1-yl]-2-oxo- ethyl}-N-pyridin-4-yl- benzamide | Compound 83 | 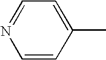 | 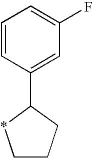 | | A | +++ |
| 4-(amino- benzylcarbamoyl- methyl)-N-(1H- pyrrolo[2,3-b]pyridin-4- yl)-benzamide | Compound 157 | 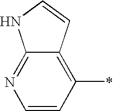 | —H | 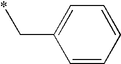 | D | nd |
| 4-(amino- benzylcarbamoyl- methyl)-N-(1H- pyrazolo[3,4-b]pyridin-4- yl)-benzamide | Compound 158 | 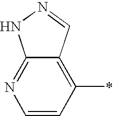 | —H | 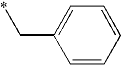 | D | nd |
| 4-(amino- benzylcarbamoyl- methyl)-N-(9H-purin-6- yl)-benzamide | Compound 159 | 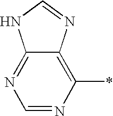 | —H | 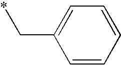 | D | nd |
| 4-[amino-(3-chloro- benzylcarbamoyl)- methyl]-N-(1H- pyrrolo[2,3-b]pyridin-4- yl)-benzamide | Compound 160 | 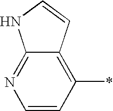 | —H | 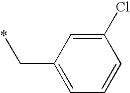 | D | nd |
| 4-[amino-(3-chloro- benzylcarbamoyl)- methyl]-N-(1H- pyrazolo[3,4-b]pyridin-4- yl)-benzamide | Compound 161 | 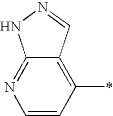 | —H | 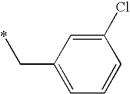 | D | nd |
| 4-[amino-(3-chloro- benzylcarbamoyl)- methyl]-N-(9H-purin-6- yl)-benzamide | Compound 162 | 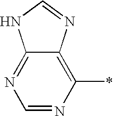 | —H | 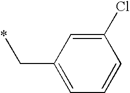 | D | nd |

TABLE 2

| | |
|---|---|
| | V20 |
| 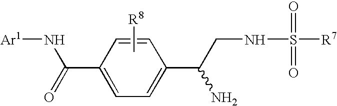 | |

| | | | | | IC₅₀ |
|---|---|---|---|---|---|
| | | | | | μM |
| Name | Compound | Ar¹— | —R⁷ | Pr | ROCK |
| 4-(1-amino-2- methanesulfonylamino- ethyl)-N-pyridin-4-yl- benzamide | Compound 55 | 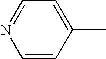 | —CH₃ | B | +++ |
| 4-(1-amino-2- ethanesulfonylamino)- ethyl)-N-pyridin-4-yl- benzamide | Compound 56 | 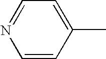 | 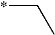 | B | +++ |
| 4-[1-amino-2-(propane-1- sulfonylamino)-ethyl]-N- pyridin-4-yl-benzamide | Compound 57 | 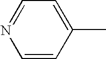 | 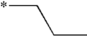 | B | +++ |
| 4-[1-amino-2-(butane-1- sulfonylamino)-ethyl]-N- pyridin-4-yl-benzamide | Compound 58 | 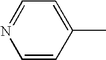 | 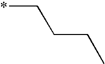 | B | +++ |
| 4-[1-amino-2-(octane-1- sulfonylamino)-ethyl]-N- pyridin-4-yl-benzamide | Compound 59 | 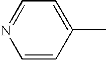 | 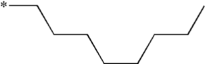 | B | ++ |
| 4-(1-amino-2- phenylmethanesulfonylamino- ethyl)-N-pyridin-4-yl- benzamide | Compound 60 | 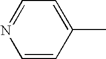 | 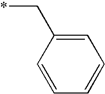 | B | +++ |
| 4-(1-amino-2- benzenesulfonylamino- ethyl)-N-pyridin-4-yl- benzamide | Compound 61 | 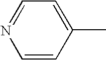 | 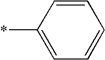 | B | +++ |
| 4-[1-amino-2-(4-methyl- benzenesulfonylamino)- ethyl]-N-pyridin-4-yl- benzamide | Compound 62 | 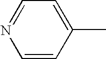 | 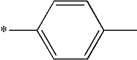 | B | +++ |
| 4-[1-amino-2-(3-methyl- benzenesulfonylamino)- ethy(]-N-pyridin-4-yl- benzamide | Compound 63 | 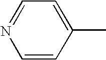 | 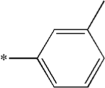 | B | +++ |
| 4-[1-amino-2-(2-methyl- benzenesulfonylamino)- ethyl]-N-pyridin-4-yl- benzamide | Compound 64 | 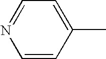 | 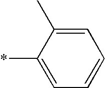 | B | +++ |
| 4-[1-amino-2-(4-fluoro- benzenesulfonylamino)- ethyl]-N-pyridin-4-yl- benzamide | Compound 65 | 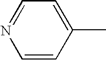 | 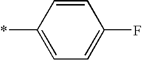 | B | +++ |
| 4-[1-amino-2-(2-fluoro- benzenesulfonylamino)- ethyl]-N-pyridin-4-yl- benzamide | Compound 66 | 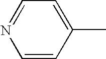 | 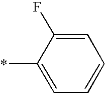 | B | +++ |
| 4-[1-amino-2-(4-chloro- benzenesulfonylamino)- ethyl]-N-pyridin-4-yl- benzamide | Compound 67 | 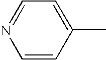 | 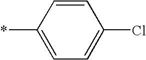 | B | +++ |
| 4-[1-amino-2-(3-chloro- benzenesulfonylamino)- ethyl]-N-pyridin-4-yl- benzamide | Compound 68 | 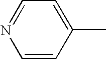 | 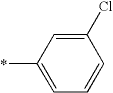 | B | +++ |
| 4-[1-amino-2-(2-chloro- benzenesulfonylamino)- ethyl]-N-pyridin-4-yl- benzamide | Compound 69 | 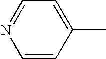 | 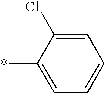 | B | +++ |
| 4-[1-amino-2-(4-methoxy- benzenesulfonylamino)- ethyl]-N-pyridin-4-yl- benzamide | Compound 70 | 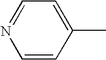 | 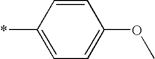 | B | +++ |
| 4-[1-amino-2-(3,4- dimethoxy- benzenesulfonylamino)- ethyl]-N-pyridin-4-yl- benzamide | Compound 71 | 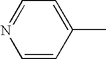 | 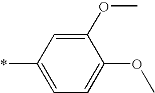 | B | +++ |
| 4-[1-amino-2-(2,5- dimethoxy- benzenesulfonylamino)- ethyl]-N-pyridin-4-yl- benzamide | Compound 72 | 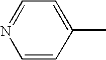 | 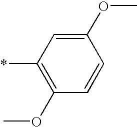 | B | +++ |
| 4-[1-amino-2-(3-phenoxy- benzenesulfonylamino)- ethyl]-N-pyridin-4-yl- benzamide | Compound 73 | 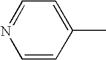 | 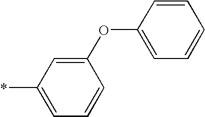 | B | ++ |
| 4-[1-amino-2-(4-phenoxy- benzenesulfonylamino)- ethyl]-N-pyridin-4-yl- benzamide | Compound 74 | 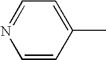 | 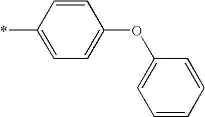 | B | ++ |
| 4-[1-amino-2-(biphenyl-3- sulfonylamino-ethyl)-N- pyridin-4-yl-benzamide | Compound 75 | 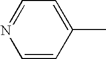 | 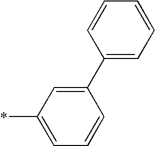 | B | ++ |
| 4-[1-amino-2-(biphenyl-4- sulfonylamino-ethyl)-N- pyridin-4-yl-benzamide | Compound 76 | 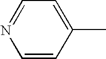 | 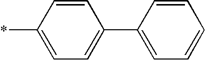 | B | ++ |
| 4-[1-amino-2- (naphthalene-1- sulfonylamino)-ethyl]-N- pyridin-4-yl-benzamide | Compound 77 | 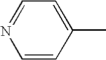 | 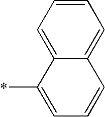 | B | +++ |
| 4-[1-amino-2-(thiophene- 2-sulfonylamino)-ethyl]-N- pyridin-4-yl-benzamide | Compound 78 | 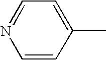 | 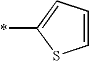 | B | +++ |
| 4-[1-amino-2-(3,5- dimethyl-isoxazole-4- sulfonylamino)-ethyl]-N- pyridin-4-yl-benzamide | Compound 79 | 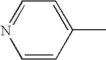 | 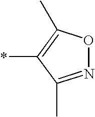 | B | +++ |
| 4-[1-amino-2-(5-chloro- 1,3-dimethyl-1H-pyrazole- 4-sulfonylamino)-ethyl]-N- pyridin-4-yl-benzamide | Compound 80 | 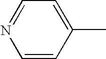 | 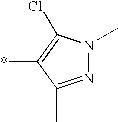 | B | ++ |
| 4-[1-amino-2- (benzo[1,2,5]-oxadiazole- 4-sulfonylamino)-ethyl]-N- pyridin-4-yl-benzamide | Compound 81 | 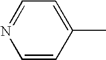 | 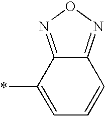 | B | +++ |
| 4-[1-amino-2- (benzo[1,2,5]-thiadiazole- 4-sulfonylamino)-ethyl]-N- pyridin-4-yl-benzamide | Compound 82 | 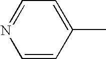 | 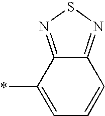 | B | +++ |
| 4-[1-amino-2-(4-methoxy- benzenesulfonylamino)- ethyl]-N-(1H-pyrrolo[2,3- b]pyridin-4-yl)-benzamide | Compound 163 | 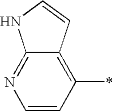 | 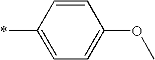 | D | nd |
| 4-[1-amino-2-(4-methoxy- benzenesulfonylamino)- ethyl]-N-(1H-pyrazolo[3,4- b]pyridin-4-yl)-benzamide | Compound 164 | 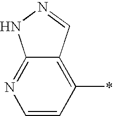 | 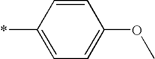 | D | nd |
| 4-[1-amino-2-(4-methoxy- benzenesulfonylamino)- ethyl]-N-(9H-purin-6-yl)- benzamide | Compound 165 | 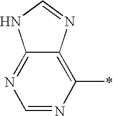 | 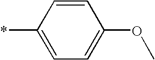 | D | nd |

TABLE 3

| | |
|---|---|
| | III20 |
| 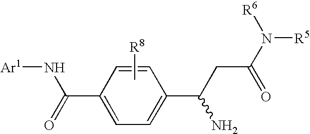 | |

| | | | | | | |
|---|---|---|---|---|---|---|
| | | | | | | IC₅₀ |
| | | | | | | μM |
| Name | Compound | Ar¹— | —R⁵ | —R⁶ | Pr | ROCK |

| | | | | | |
|---|---|---|---|---|---|
| 4-[1-amino-3-oxo-3- (2-phenyl-pyrrolidin- 1-yl)-propyl]-N- pyridin-4-yl- benzamide | Compound 84 | 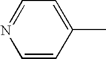 | 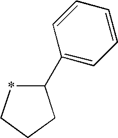 | C | +++ |
| 4-{1-amino-3-[2-(3- chloro-phenyl)- pyrrolidin-1-yl]-3- oxo-propyl}-N- pyridin-4-yl- benzamide | Compound 85 | 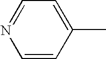 | 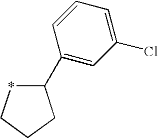 | C | +++ |
| 4-{1-amino-3-[2-(4- fluoro-phenyl)- pyrrolidin-1-yl]-3- oxo-propyl}-N- pyridin-4-yl- benzamide | Compound 86 | 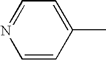 | 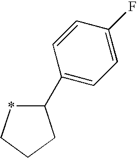 | C | +++ |
| 4-{1-amino-3-[2-(3- fluoro-phenyl)- pyrrolidin-1-yl]-3- oxo-propyl}-N- pyridin-4-yl- benzamide | Compound 87 | 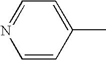 | 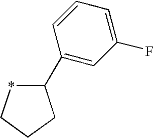 | C | +++ |
| 4-{1-amino-3-[2- (2,5-difluoro- phenyl)-pyrrolidin-1- yl]-3-oxo-propyl}-N- pyridin-4-yl- benzamide | Compound 88 | 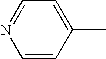 | 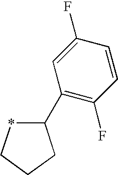 | C | +++ |
| 4-{1-amino-3-[2- (2,4-difluoro- phenyl)-pyrrolidin-1- yl]-3-oxo-propyl}-N- pyridin-4-yl- benzamide | Compound 89 | 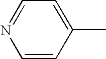 | 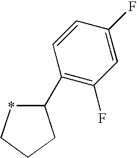 | C | +++ |
| 4-{1-amino-3-[2-(4- chloro-phenyl)- pyrrolidin-1-yl]-3- oxo-propyl}-N- pyridin-4-yl- benzamide | Compound 90 | 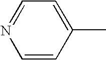 | 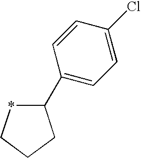 | C | +++ |
| 4-{1-amino-3-[2- (3,4-dichloro- phenyl)-pyrrolidin-1- yl]-3-oxo-propyl}-N- pyridin-4-yl- benzamide | Compound 91 | 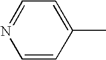 | 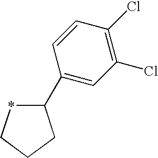 | C | +++ |
| 4-[1-amino-3-oxo-3- (2-phenyl-piperidin- 1-yl)-propyl]-N- pyridin-4-yl- benzamide | Compound 92 | 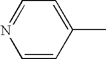 | 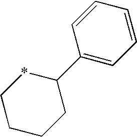 | C | +++ |
| 4-[1-amino-3-(2- naphthalen-1-yl- piperidin-1-yl)-3- oxo-propyl]-N- pyridin-4-yl- benzamide | Compound 93 | 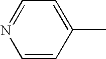 | 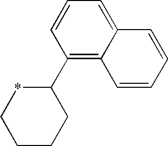 | C | +++ |
| 4-{1-amino-3-[2-(4- chloro-phenyl)- piperidin-1-yl]-3- oxo-propyl}-N- pyridin-4-yl- benzamide | Compound 94 | 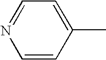 | 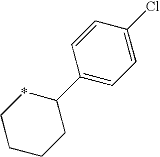 | C | +++ |
| 4-{1-amino-3-[2- (2,5-dichloro- phenyl)-piperidin-1- yl]-3-oxo-propyl}-N- pyridin-4-yl- benzamide | Compound 95 | 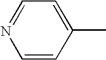 | 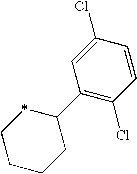 | C | +++ |
| 4-{1-amino-3-[2-(5- chloro-2-methoxy- phenyl)-piperidin-1- yl]-3-oxo-propyl}-N- pyridin-4-yl- benzamide | Compound 96 | 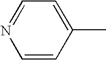 | 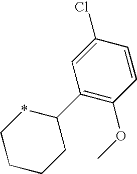 | C | +++ |
| 4-{1-amino-3-[2-(4- fluoro-phenyl)- piperidin-1-yl]-3- oxo-propyl}-N- pyridin-4-yl- benzamide | Compound 97 | 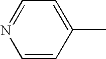 | 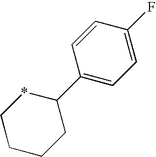 | C | +++ |
| 4-{1-amino-3-[2-(3- methoxy-phenyl)- piperidin-1-yl]-3- oxo-propyl}-N- pyridin-4-yl- benzamide | Compound 98 | 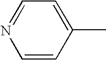 | 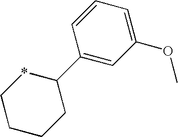 | C | +++ |
| 4-[1-Amino-3-oxo-3- (3-phenyl- morpholin-4-yl)- propyl]-N-pyridin-4- yl-benzamide | Compound 99 | 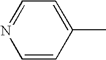 | 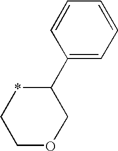 | C | +++ |
| 4-{1-amino-3-[3-(4- chloro-phenyl)- morpholin-4-yl]-3- oxo-propyl}-N- pyridin-4-yl- benzamide | Compound 100 | 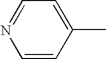 | 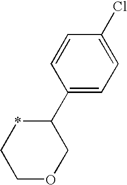 | C | +++ |
| 4-{1-amino-3-[3-(4- fluoro-phenyl)- morpholin-4-yl]-3- oxo-propyl}-N- pyridin-4-yl- benzamide | Compound 101 | 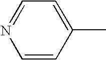 | 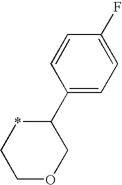 | C | +++ |
| 4-[1-amino-3-(5- chloro-3,4-dihydro- 1H-isoquinolin-2-yl)- 3-oxo-propyl]-N- pyridin-4-yl- benzamide | Compound 102 | 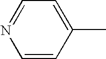 | 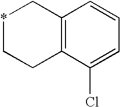 | C | +++ |
| 4[1-amino-3-(7- chloro-3,4-dihydro- 1H-isoquinolin-2-yl)- 3-oxo-propyl]-N- pyridin-4-yl- benzamide | Compound 103 | 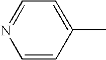 | 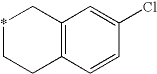 | C | +++ |
| 4-[1-amino-3-(7- fluoro-3,4-dihydro- 1H-isoquinolin-2-yl)- 3-oxo-propyl]-N- pyridin-4-yl- benzamide | Compound 104 | 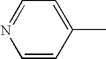 | 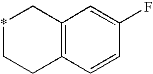 | C | +++ |
| 4-[1-amino-3-(1,3- dihydro-isoindol-2- yl)-3-oxo-propyl]-N- pyridin-4-yl- benzamide | Compound 105 | 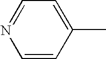 | 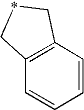 | C | +++ |
| 4-(1-amino-3-oxo-3- thiazolidin-3-yl- propyl)-N-pyridin-4- yl-benzamide | Compound 106 | 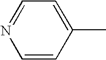 | 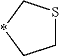 | A | +++ |
| 4-(1-amino-3-oxo-3- piperidin-1-yl- propyl)-N-pyridin-4- yl-benzamide | Compound 107 | 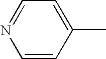 | 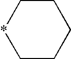 | A | +++ |
| 4-[1-amino-3-(3,5- dimethyl-piperidin- 1-yl)-3-oxo-propyl]- N-pyridin-4-yl- benzamide | Compound 108 | 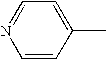 | 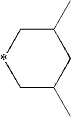 | A | +++ |
| 4-[1-amino-3-(3- hydroxymethyl- piperidin-1-yl)-3- oxo-propyl]-N- pyridin-4-yl- benzamide | Compound 109 | 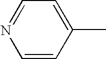 | 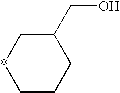 | A | +++ |
| 4-[1-amino-3- (octahydro-quinolin- 1-yl)-3-oxo-propyl]- N-pyridin-4-yl- benzamide | Compound 110 | 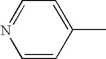 | 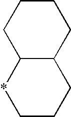 | A | +++ |
| 4-[1-amino-3-(4- hydroxy-4-phenyl- piperidin-1-yl)-3- oxo-propyl]-N- pyridin-4-yl- benzamide | Compound 111 | 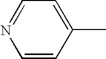 | 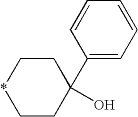 | A | +++ |
| 4-(1-amino-3- morpholin-4-yl-3- oxo-propyl)-N- pyridin-4-yl- benzamide | Compound 112 | 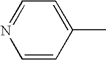 | 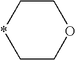 | A | +++ |
| 4-[1-amino-3-(2,6- dimethyl-morpholin- 4-yl)-3-oxo-propyl]- N-pyridin-4-yl- benzamide | Compound 113 | 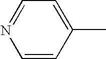 | 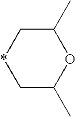 | A | +++ |
| 4-[1-amino-3-(4- isopropyl-piperazin- 1-yl)-3-oxo-propyl]- N-pyridin-4-yl- benzamide | Compound 114 | 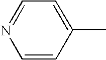 | 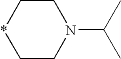 | A | +++ |
| 4-{1-amino-3-[4-(2- hydroxy-ethyl)- piperazin-1-yl]-3- oxo-propyl}-N- pyridin-4-yl- benzamide | Compound 115 | 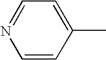 | 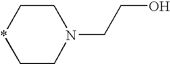 | A | +++ |
| 4-[1-amino-3-oxo-3- (4-phenyl-piperazin- 1-yl)-propyl]-N- pyridin-4-yl- benzamide | Compound 116 | 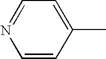 | 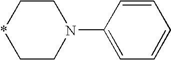 | A | +++ |
| 4-[1-amino-3-(4- phenyl-piperidin-1- yl)-3-oxo-propyl]-N- pyridin-4-yl- benzamide | Compound 117 | 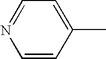 | 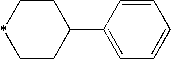 | A | +++ |
| 4-{1-amino-3[4-(4- fluoro-phenyl)-3,6- dihydro-2H-pyridin- 1-yl]-3-oxo-propyl}- N-pyridin-4-yl- benzamide | Compound 118 | 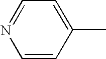 | 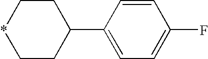 | A | +++ |
| 4-[1-amino-3-(4- benzyl-piperidin-1- yl)-3-oxo-propyl]-N- pyridin-4-yl- benzamide | Compound 119 | 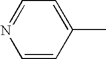 | 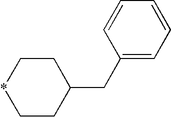 | A | +++ |
| 4-[1-amino-3-(4- benzooxazol-2-yl- piperidin-1-yl)-3- oxo-propyl]-N- pyridin-4-yl- benzamide | Compound 120 | 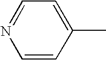 | 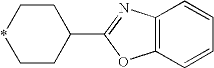 | A | +++ |
| 4-{1-Amino-3-[4-(4- methoxy-benzyl)- piperazin-1-yl]-3- oxo-propyl}-N- pyridin-4-yl- benzamide | Compound 121 | 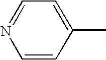 | 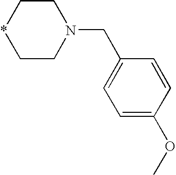 | A | ++ |
| 4-{1-amino-3-[4-(2- fluoro-benzyl)- piperazin-1-yl]-3- oxo-propyl}-N- pyridin-4-yl- benzamide | Compound 122 | 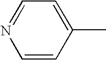 | 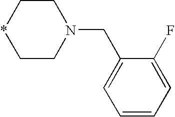 | A | +++ |
| 4-{1-amino-3-[4-(4- fluoro-benzyl)- piperazin-1-yl]-3- oxo-propyl}-N- pyridin-4-yl- benzamide | Compound 123 | 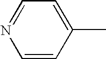 | 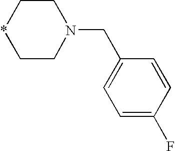 | A | +++ |
| 4-{1-amino-3-[4- (furan-2-carbonyl)- piperazin-1-yl]-3- oxo-propyl}-N- pyridin-4-yl- benzamide | Compound 124 | 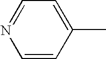 | 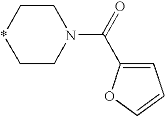 | A | +++ |
| 4-{1-amino-3-[4- (1H-indol-3-yl)- piperidin-1-yl]-3- oxo-propyl}-N- pyridin-4-yl- benzamide | Compound 125 | 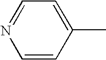 | 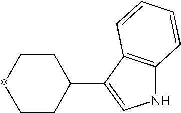 | A | +++ |
| 2-{3-amino-3-[4- (pyridin-4- ylcarbamoyl)- phenyl]-propionyl}- 1,2,3,4-tetrahydro- isoquinoline-6- carboxylic acid methyl ester | Compound 126 | 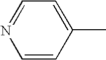 | 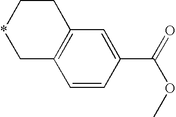 | A | ++ |
| 4(1-{3-amino-3-[4- (pyridin-4- ylcarbamoyl)- phenyl]-propionyl}- piperidin-2-yl)- benzoic acid methyl ester | Compound 127 | 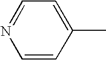 | 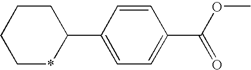 | A | +++ |
| 4-[1-amino-3-oxo- (1,3,4,9-tetrahydro- β-carbolin-2-yl)- propyl]-N-pyridin-4- yl-benzamide | Compound 128 | 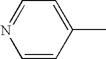 | 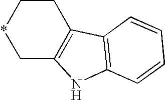 | A | +++ |
| 4-{1-amino-3-[1-(3- chloro-phenyl)- 1,3,4,9-tetrahydro- β-carbolin-2-yl]-3- oxo-propyl}-N- pyridin-4-yl- benzamide | Compound 129 | 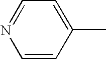 | 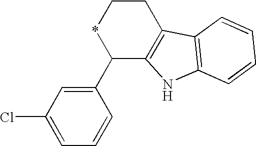 | A | ++ |
| 4-{1-amino-3-[1-(3- bromo-4-methoxy- phenyl)-1,3,4,9- tetrahydro-β- carbolin-2-yl]-3-oxo- propyl}-N-pyridin-4- yl-benzamide | Compound 130 | 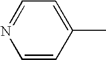 | 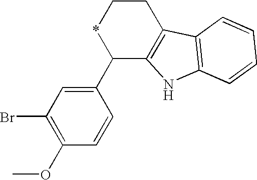 | A | ++ |
| 4-[1-amino-3-(3,4- dihydro-1H- isoquinolin-2-yl)-3- oxo-propyl]-N- pyridin-4-yl- benzamide | Compound 131 | 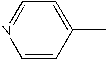 | 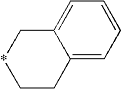 | A | +++ |
| 4-{1-amino-3-[2-(3- fluoro-phenyl)- pyrrolidin-1-yl]-3- oxo-propyl}-N-(1H- pyrrolo[2,3- b]pyridin-4-yl)- benzamide | Compound 166 | 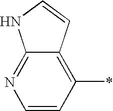 | 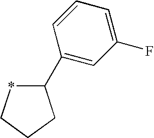 | D | nd |
| 4-{1-amino-3-[2-(3- fluoro-phenyl)- pyrrolidin-1-yl]-3- oxo-propyl}-N-(1H- pyrazolo[3,4- b]pyridin-4-yl)- benzamide | Compound 167 | 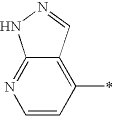 | 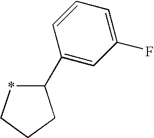 | D | nd |
| 4-{1-amino-3-[2-(3- fluoro-phenyl)- pyrrolidin-1-yl]-3- oxo-propyl}-N-(9H- purin-6-yl)- benzamide | Compound 168 | 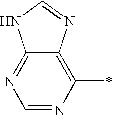 | 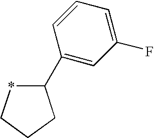 | D | nd |

### Compound 132: 4-{Amino-[1-(4-methoxy-benzenesulfonyl)-pyrrolidin-2-yl]-methyl}-N-pyridin-4-yl-benzamide dihydrochloric acid salt

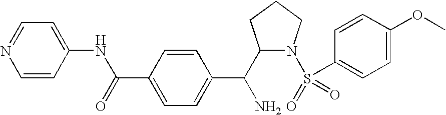

To a solution of Intermediate 5 (25 mg) in dry THF (1 ml), were added the 4-methoxy-benzenesulfonyl chloride (1.1 eq.) and DIEA (1.2 eq.). The reaction mixture was stirred at RT for 3 hours and then was diluted with EtOAc and water. The product was extracted with EtOAc. The organic layer was dried over MgSO4, filtered and evaporated. The crude product was purified by filtration through a silica gel pad (DCM/MeOH 95/5). The product was dissolved in dry 1,4-dioxane, and HCl was bubbled in the solution for 10 minutes. The solvent was evaporated to afford the title compound as a white powder (71% overall yield).

### Compound 133: 4-{Amino-[1-(2-chloro-benzenesulfonyl)-pyrrolidin-2-yl]-methyl}-N-pyridin-4-yl-benzamide dihydrochloric acid salt

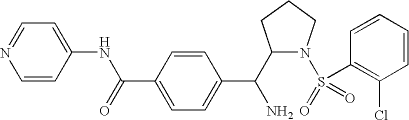

Compound 133 was prepared according to the procedure described for Compound 132, starting from Intermediate 5 and 2-chloro-benzenesulfonyl chloride (27% yield, pale yellow powder).

### Compound 134: 4-{Amino-[1-(3-chloro-benzenesulfonyl)-pyrrolidin-2-yl]-methyl}-N-pyridin-4-yl-benzamide dihydrochloric acid salt

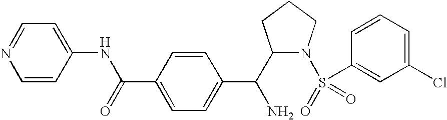

Compound 134 was prepared according to the procedure described for Compound 132, starting from Intermediate 5 and 3-chloro-benzenesulfonyl chloride (51% yield, pale yellow powder).

### Compound 135: 4-{Amino-[1-(4-chloro-benzenesulfonyl)-pyrrolidin-2-yl]-methyl}-N-pyridin-4-yl-benzamide dihydrochloric acid salt

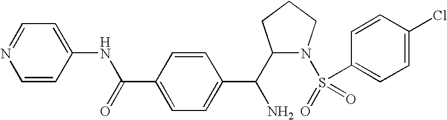

Compound 135 was prepared according to the procedure described for Compound 132, starting from Intermediate 5 and 4-chloro-benzenesulfonyl chloride (66% yield, pale yellow powder).

### Compound 136: 4-{Amino-[1-(2-fluoro-benzenesulfonyl)-pyrrolidin-2-yl]-methyl}-N-pyridin-4-yl-benzamide dihydrochloric acid salt

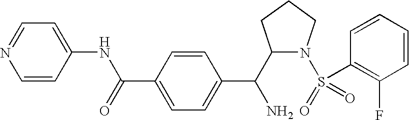

Compound 136 was prepared according to the procedure described for Compound 132, starting from Intermediate 5 and 2-fluoro-benzenesulfonyl chloride (68% yield, white powder).

### Compound 137: 4-{Amino-[1-(3-fluoro-benzenesulfonyl)-pyrrolidin-2-yl]-methyl}-N-pyridin-4-yl-benzamide dihydrochloric acid salt

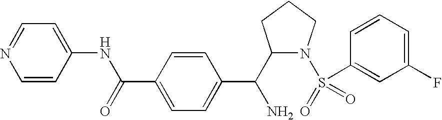

Compound 137 was prepared according to the procedure described for Compound 132, starting from Intermediate 5 and 3-fluoro-benzenesulfonyl chloride (68% yield, white powder).

### Compound 138: 4-{Amino-[1-(4-fluoro-benzenesulfonyl)-pyrrolidin-2-yl]-methyl}-N-pyridin-4-yl-benzamide dihydrochloric acid salt

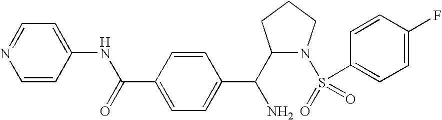

Compound 138 was prepared according to the procedure described for Compound 132, starting from Intermediate 5 and 4-fluoro-benzenesulfonyl chloride (68% yield, white powder).

### Compound 139: 4-[1-Amino-2-(benzenesulfonyl-methyl-amino)-ethyl]-N-pyridin-4-yl-benzamide dihydrochloric acid salt

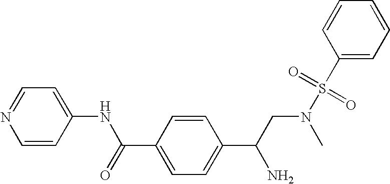

To a solution of Intermediate 6 (75 mg) in dry THF (1 ml), were added the benzenesulfonyl chloride (1.1 eq.) and DIEA (1.2 eq.). The reaction mixture was stirred at RT for 1 hour and then was diluted with EtOAc and water. The product was extracted with EtOAc. The organic layer was dried over MgSO4, filtered and evaporated. The product was dissolved in dry 1,4-dioxane, and HCl was bubbled in the solution for 10 minutes. The solvent was evaporated to afford the title compound as a white powder (74% yield).

### Compound 140: 4-{1-Amino-2-[(4-fluoro-benzenesulfonyl)-methyl-amino]-ethyl}-N-pyridin-4-yl-benzamide dihydrochloric acid salt

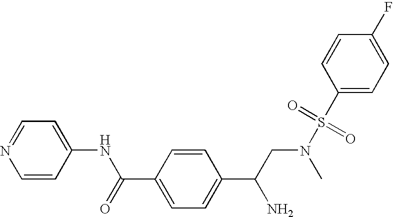

Compound 140 was prepared according to the procedure described for Compound 139, starting from Intermediate 6 and 4-fluoro-benzenesulfonyl chloride (70% yield, white powder).

### Compound 141: 4-{1-Amino-2-[(3-fluoro-benzenesulfonyl)-methyl-amino]-ethyl}-N-pyridin-4-yl-benzamide dihydrochloric acid salt

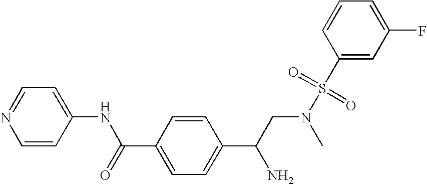

Compound 141 was prepared according to the procedure described for Compound 139, starting from Intermediate 6 and 3-fluoro-benzenesulfonyl chloride (90% yield, white powder).

### Compound 142: 4-{1-Amino-2-[(2-fluoro-benzenesulfonyl)-methyl-amino]-ethyl}-N-pyridin-4-yl-benzamide dihydrochloric acid salt

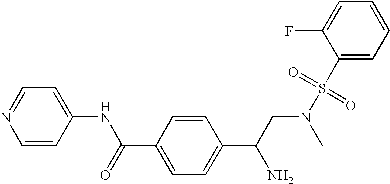

Compound 142 was prepared according to the procedure described for Compound 139, starting from Intermediate 6 and 2-fluoro-benzenesulfonyl chloride (90% yield, white powder).

### Compound 143: 4-{1-Amino-2-[(2-chloro-benzenesulfonyl)-methyl-amino]-ethyl}-N-pyridin-4-yl-benzamide dihydrochloric acid salt

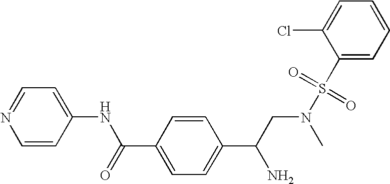

Compound 143 was prepared according to the procedure described for Compound 139, starting from Intermediate 6 and 2-chloro-benzenesulfonyl chloride (92% yield, white powder).

### Compound 144: 4-{1-Amino-2-[(3-chloro-benzenesulfonyl)-methyl-amino]-ethyl}-N-pyridin-4-yl-benzamide dihydrochloric acid salt

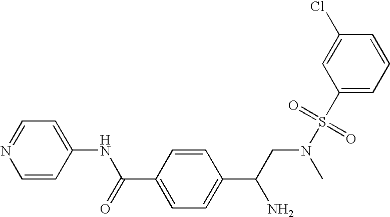

Compound 144 was prepared according to the procedure described for Compound 139, starting from Intermediate 6 and 3-chloro-benzenesulfonyl chloride (90% yield, white powder).

### Compound 145: 4-{1-Amino-2-[(4-chloro-benzenesulfonyl)-methyl-amino]-ethyl}-N-pyridin-4-yl-benzamide dihydrochloric acid salt

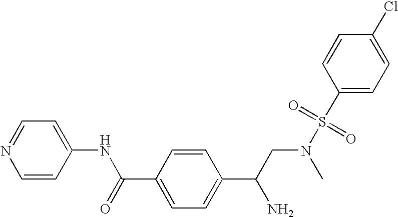

Compound 145 was prepared according to the procedure described for Compound 139, starting from Intermediate 6 and 4-chloro-benzenesulfonyl chloride (90% yield, white powder).

### Compound 146: 4-{1-Amino-2-[(4-methoxy-benzenesulfonyl)-methyl-amino]-ethyl}-N-pyridin-4-yl-benzamide dihydrochloric acid salt

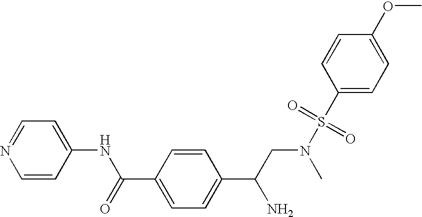

Compound 146 was prepared according to the procedure described for Compound 139, starting from Intermediate 6 and 4-chloro-benzenesulfonyl chloride (92% yield, white powder).

Unless indicated otherwise, percentage of enantiomeric (or diastereisomeric) excess was determined by chiral HPLC. As used herein the term “Tret” means “retention time”.

### Compound 147: 4-[(R)-1-Amino-3-(7-fluoro-3,4-dihydro-1H-isoquinolin-2-yl)-3-oxo-propyl]-N-pyridin-4-yl-benzamide dihydrochloric acid salt

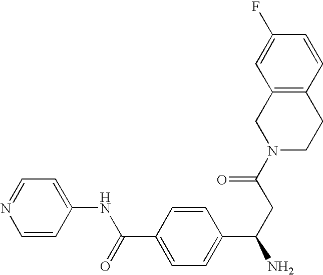

Compound 147 was prepared according to the protocol C starting from Intermediate 4 and 7-fluoro-1,2,3,4-tetrahydro-isoquinoline (44% yield, white powder).

% ee=100% (chiral HPLC: column OJ-H, 0.46×250 mm, hexane/2-propanol 85/15 with 0.1% DIPEA, Tret: 62 min).

### Compound 148: 4-[(S)-1-Amino-3-(7-fluoro-3,4-dihydro-1H-isoquinolin-2-yl)-3-oxo-propyl]-N-pyridin-4-yl-benzamide dihydrochloric acid salt

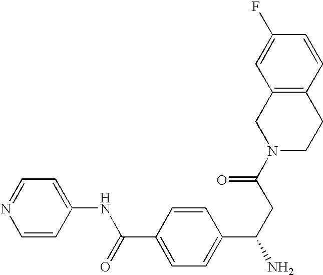

Compound 148 was prepared according to the protocol C starting from Intermediate 5 and 7-fluoro-1,2,3,4-tetrahydro-isoquinoline (45% yield, white powder).

% ee=95% (chiral HPLC: column OJ-H, 0.46×250 mm, hexane/2-propanol 85/15 with 0.1% DIPEA, Tret: 55 min).

### Compound 149: 4-{(R)-1-Amino-3-[(R)-2-(3-fluoro-phenyl)-pyrrolidin-1-yl]-3-oxo-propyl}-N-pyridin-4-yl-benzamide dihydrochloric acid salt

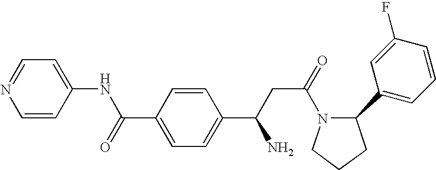

Compound 149 was prepared according to the protocol C starting from Intermediate 4 and (R)-2-(3-fluoro-phenyl)-pyrrolidine (47% yield, pale brown powder).

% ee=100%; % de=94% (chiral HPLC: column AD-H, 0.46×250 mm, hexane/ethanol 85/15 with 0.1% DIPEA, Tret: 43 min).

### Compound 150: 4-{(R)-1-Amino-3-[(S)-2-(3-fluoro-phenyl)-pyrrolidin-1-yl]-3-oxo-propyl}-N-pyridin-4-yl-benzamide dihydrochloric acid salt

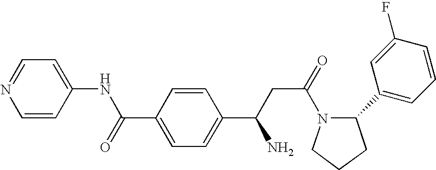

Compound 150 was prepared according to the protocol C starting from Intermediate 4 and (S)-2-(3-fluoro-phenyl)-pyrrolidine (47% yield, pale brown powder).

% ee=96%; % de=94% (chiral HPLC: column AD-H, 0.46×250 mm, hexane ethanol 85/15 with 0.1% DIPEA, Tret: 123 min).

### Compound 151: 4-{(S)-1-Amino-3-[(R)-2-(3-fluoro-phenyl)-pyrrolidin-1-yl]-3-oxo-propyl}-N-pyridin-4-yl-benzamide dihydrochloric acid salt

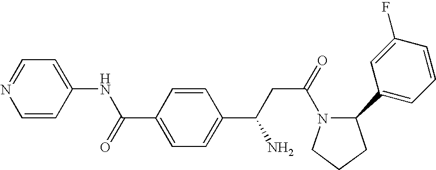

Compound 151 was prepared according to the protocol C starting from Intermediate 5 and (R)-2-(3-fluoro-phenyl)-pyrrolidine (59% yield, pale brown powder).

% ee=100%; % de=95° A) (chiral HPLC: column AD-H, 0.46×250 mm, hexane/ethanol 85/15 with 0.1% DIPEA, Tret: 36 min).

### Compound 152: 4-{(S)-1-Amino-3-[(S)-2-(3-fluoro-phenyl)-pyrrolidin-1-yl]-3-oxo-propyl}-N-pyridin-4-yl-benzamide dihydrochloric acid salt

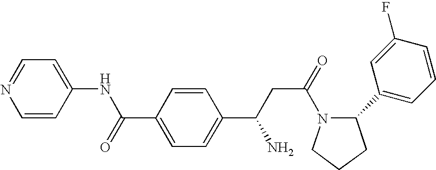

Compound 152 was prepared according to the protocol C starting from Intermediate 5 and (S)-2-(3-fluoro-phenyl)-pyrrolidine (45% yield, pale brown powder).

% ee=97%; ° A) de=96% (chiral HPLC: column AD-H, 0.46×250 mm, hexane/ethanol 85/15 with 0.1% DIPEA, Tret: 78 min).

### Compound 153: 4-{(R)1-Amino-2-[(4-methoxy-benzenesulfonyl)-methyl-amino]-ethyl}-N-pyridin-4-yl-benzamide dihydrochloric acid salt

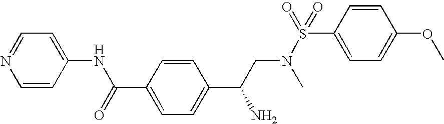

Compound 153 was obtained by preparative chiral HPLC separation of Compound 146 (Column: AD-H, 10×250 mm, 5 μm; Solvent: hexane/ethanol 80/20 with 0.1% DIPEA).

% ee=100% (chiral HPLC: column AD-H, 0.46×250 mm, hexane/ethanol 80/20 with 0.1% DIPEA, Tᵣₑₜ: 70 min).

### Compound 154: 4-{(S)1-Amino-2-[(4-methoxy-benzenesulfonyl)-methyl-amino]-ethyl}-N-pyridin-4-yl-benzamide dihydrochloric acid salt

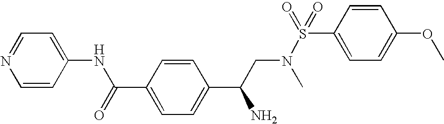

Compound 154 was obtained by preparative chiral HPLC separation of Compound 146 (Column: AD-H, 10×250 mm, 5 μm; Solvent: hexane/ethanol 80/20 with 0.1% DIPEA).

% ee=100% (chiral HPLC: column AD-H, 0.46×250 mm, hexane/ethanol 80/20 with 0.1% DIPEA, Tᵣₑₜ: 62 min).

### Compound 155: 4-((S)-Amino-benzylcarbamoyl-methyl)-N-pyridin-4-yl-benzamide dihydrochloric acid salt

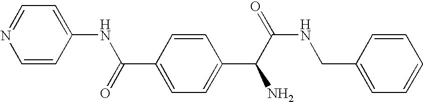

Compound 155 was obtained by preparative chiral HPLC separation of Compound 1 (Column: AD-H, 10×250 mm, 5 μm; Solvent: hexane/ethanol 80/20 with 0.1% DIPEA).

% ee=100% (chiral HPLC: column AD-H, 0.46×250 mm, hexane/ethanol 80/20 with 0.1% DIPEA, Tᵣₑₜ: 29.6 min).

### Compound 156: 4-((R)-Amino-benzylcarbamoyl-methyl)-N-pyridin-4-yl-benzamide dihydrochloric acid salt

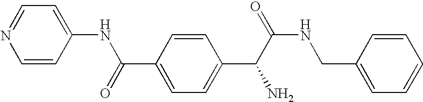

Compound 156 was obtained by preparative chiral HPLC separation of Compound 1 (Column: AD-H, 10×250 mm, 5 μm; Solvent: hexane/ethanol 80/20 with 0.1% DIPEA).

% ee=99.6% (chiral HPLC: column AD-H, 0.46×250 mm, hexane/ethanol 80/20 with 0.1% DIPEA, Tᵣₑₜ: 53.7 min).

Table 4 shows the results for compounds 132 to 156. As used herein the term “ND” means “not determined yet”.

TABLE 4

| | | IC_{50 μM} |
|---|---|---|
| Name | Compound | ROCK |
| 4-{Amino-[1-(4-methoxy-benzenesulfonyl)-pyrrolidin-2-yl]- | Compound 132 | ++ |
| methyl}-N-pyridin-4-yl-benzamide | | |
| 4-{Amino-[1-(2-chloro-benzenesulfonyl)-pyrrolidin-2-yl]- | Compound 133 | ++ |
| methyl}-N-pyridin-4-yl-benzamide | | |
| 4-{Amino-[1-(3-chloro-benzenesulfonyl)-pyrrolidin-2-yl]- | Compound 134 | ++ |
| methyl}-N-pyridin-4-yl-benzamide | | |
| 4-{Amino-[1-(4-chloro-benzenesulfonyl)-pyrrolidin-2-yl]- | Compound 135 | ++ |
| methyl}-N-pyridin-4-yl-benzamide | | |
| 4-{Amino-[1-(2-fluoro-benzenesulfonyl)-pyrrolidin-2-yl]- | Compound 136 | ++ |
| methyl}-N-pyridin-4-yl-benzamide | | |
| 4-{Amino-[1-(3-fluoro-benzenesulfonyl)-pyrrolidin-2-yl]- | Compound 137 | ++ |
| methyl}-N-pyridin-4-yl-benzamide | | |
| 4-{Amino-[1-(4-fluoro-benzenesulfonyl)-pyrrolidin-2-yl]- | Compound 138 | ++ |
| methyl}-N-pyridin-4-yl-benzamide | | |
| 4-[1-Amino-2-(benzenesulfonyl-methyl-amino)-ethyl]-N- | Compound 139 | ++ |
| pyridin-4-yl-benzamide | | |
| 4-{1-Amino-2-[(4-fluoro-benzenesulfonyl)-methyl-amino]- | Compound 140 | ++ |
| ethyl}-N-pyridin-4-yl-benzamide | | |
| 4-{1-Amino-2-[(3-fluoro-benzenesulfonyl)-methyl-amino]- | Compound 141 | ++ |
| ethyl}-N-pyridin-4-yl-benzamide | | |
| 4-{1-Amino-2-[(2-fluoro-benzenesulfonyl)-methyl-amino]- | Compound 142 | ++ |
| ethyl}-N-pyridin-4-yl-benzamide | | |
| 4-{1-Amino-2-[(2-chloro-benzenesulfonyl)-methyl-amino]- | Compound 143 | ++ |
| ethyl}-N-pyridin-4-yl-benzamide | | |
| 4-{1-Amino-2-[(3-chloro-benzenesulfonyl)-methyl-amino]- | Compound 144 | ++ |
| ethyl}-N-pyridin-4-yl-benzamide | | |
| 4-{1-Amino-2-[(4-chloro-benzenesulfonyl)-methyl-amino]- | Compound 145 | ++ |
| ethyl}-N-pyridin-4-yl-benzamide | | |
| 4-{1-Amino-2-[(4-methoxy-benzenesulfonyl)-methyl-amino]- | Compound 146 | +++ |
| ethyl}-N-pyridin-4-yl-benzamide | | |
| 4-[(R)-1-Amino-3-(7-fluoro-3,4-dihydro-1H-isoquinolin-2-yl)-3- | Compound 147 | nd |
| oxo-propyl]-N-pyridin-4-yl-benzamide | | |
| 4-[(S)-1-Amino-3-(7-fluoro-3,4-dihydro-1H-isoquinolin-2-yl)-3- | Compound 148 | nd |
| oxo-propyl]-N-pyridin-4-yl-benzamide | | |
| 4-{(R)-1-Amino-3-[(R)-2-(3-fluoro-phenyl)-pyrrolidin-1-yl]-3- | Compound 149 | +++ |
| oxo-propyl}-N-pyridin-4-yl-benzamide | | |
| 4-{(R)-1-Amino-3-[(S)-2-(3-fluoro-phenyl)-pyrrolidin-1-yl]-3- | Compound 150 | +++ |
| oxo-propyl}-N-pyridin-4-yl-benzamide | | |
| 4-{(S)-1-Amino-3-[(R)-2-(3-fluoro-phenyl)-pyrrolidin-1-yl]-3- | Compound 151 | +++ |
| oxo-propyl}-N-pyridin-4-yl-benzamide | | |
| 4-{(S)-1-Amino-3-[(S)-2-(3-fluoro-phenyl)-pyrrolidin-1-yl]-3- | Compound 152 | +++ |
| oxo-propyl}-N-pyridin-4-yl-benzamide | | |
| 4-{(R)1-Amino-2-[(4-methoxy-benzenesulfonyl)-methyl-amino]- | Compound 153 | + |
| ethyl}-N-pyridin-4-yl-benzamide | | |
| 4-{(S)1-Amino-2-[(4-methoxy-benzenesulfonyl)-methyl-amino]- | Compound 154 | +++ |
| ethyl}-N-pyridin-4-yl-benzamide | | |
| 4-((S)-Amino-benzylcarbamoyl-methyl)-N-pyridin-4-yl- | Compound 155 | +++ |
| benzamide | | |
| 4-((R)-Amino-benzylcarbamoyl-methyl)-N-pyridin-4-yl- | Compound 156 | ++ |
| benzamide | | |

TABLE 5

| | |
|---|---|
| | IV2 |
| 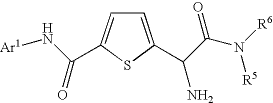 | |

| | | | | | | IC₅₀ |
|---|---|---|---|---|---|---|
| | | | | | | μM |
| Name | Compound | Ar¹— | —R⁵ | —R⁶ | Pr | ROCK |
| 5-(Amino- benzylcarbamoyl-methyl)- thiophene-2-carboxylic acid pyridin-4-ylamide | Compound 169 | 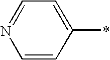 | —H | 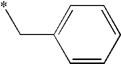 | C | nd |
| 5-[Amino-(3-chloro- benzylcarbamoyl)-methyl]- thiophene-2-carboxylic acid pyridin-4-ylamide | Compound 170 | 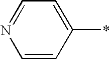 | —H | 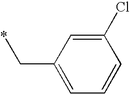 | C | nd |

TABLE 6

| | |
|---|---|
| | V2 |
| 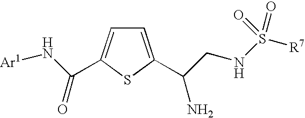 | |

| | | | | | IC₅₀ |
|---|---|---|---|---|---|
| | | | | | μM |
| Name | Compound | Ar¹— | —R⁷ | Pr | ROCK |
| 5-[1-Amino-2-(4-methoxy- benzenesulfonylamino)- ethyl]-thiophene-2- carboxylic acid pyridin-4- ylamide | Compound 171 | 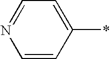 | 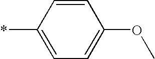 | B | nd |

TABLE 7

| | |
|---|---|
| | III21 |
| 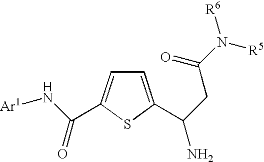 | |

| | | | | | | |
|---|---|---|---|---|---|---|
| | | | | | | IC₅₀ |
| | | | | | | μM |
| Name | Compound | Ar¹— | —R⁵ | —R⁶ | Pr | ROCK |

| | | | | | |
|---|---|---|---|---|---|
| 5-{1-Amino-3-[2-(3- fluoro-phenyl)- pyrrolidin-1-yl]-3- oxo-propyl}- thiophene-2- carboxylic acid pyridin-4-ylamide | Compound 172 | 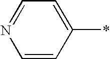 | 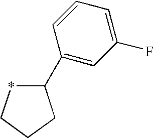 | D | nd |

### Example 2

Compounds according to the invention showed improved (compared to known ROCK inhibitors) cellular potency, excellent selectivity, good physicochemical and pharmacokinetic properties.

Compounds of the invention are ATP-competitive kinase inhibitors. As ROCK inhibitors, they can be used in the modulation of smooth muscle cell contractility, cell motility, apoptosis, axon growth and regeneration and metastasis formation. For example, they can be used in the treatment of pulmonary vasoconstriction, vascular disease, cerebral vasospasm, erectile dysfunction (ED), glaucoma, cell transformation, cancer metastasis, fibrosis, and cerebral vasospasm.

In comparison to known ROCK inhibitors, like fasudil and Y-27632, compounds of the invention showed similar or improved potency (20-100 fold), improved selectivity against other kinases (more than 10 fold) and enhanced physicochemical and pharmacokinetic properties (allowing systemic exposure via oral administration for the intended medical use). Additional routes of administration are also enabled.

In the treatment of glaucoma, ROCK inhibition with compounds of the invention impairs the actin-myosin interactions that regulate smooth muscle contraction of the ciliary muscle and especially the trabecular meshwork in the eye. This improves the aqueous humor outflow and reduces intraocular pressure.

The present ROCK inhibitors can be used to decrease apoptosis and necrosis in tissues subjected to neuronal damage. These effects are beneficial to late stage glaucoma patients, but also for the treatment of degenerative retinal diseases such as age-related macular degeneration (AMD) and retinitis pigmentosa (RP).

### Biochemical Profile

### Potency and Selectivity in Initial Kinase Panel

Potency and selectivity of compounds of the invention to closely related kinases was evaluated (data not shown).

Compared with known ROCK inhibitors such as Y-27632, compounds of the invention are at least as potent or 10× more potent and have improved selectivity versus closely related protein kinases of the AGC-family such as, PKA, PKB and PKC.

### Specificity

Compounds of the invention were also tested for specificity on a panel of 21 receptors, ion channels and transporters at 10 μM (data not shown). For the specificity panel, representative sets of neurotransmitter- and hormone receptors, ion channels and neurotransmitter transporters were selected.

The data obtained with this panel supported a good specificity with the potential for minimal pharmacological side-effects of the profiled compounds.

It was shown that compounds of the invention are potent, selective and specific ROCK inhibitor.

### Cell Based Profile

Kinase inhibitors generally show a loss in potency between biochemical assays and cellular assays due to cell penetration and differences in ATP concentrations.

To have a more accurate perspective on the selectivity of the present ROCK inhibitors, the in vitro biochemical data of compounds of the invention were complemented with in vitro cellular IC50-values.

A cellular assay was configured measuring ROCK activity in a physiological context.

The assay is based on the observation that lipopolysaccharide (LPS) induced TNFα release from human monocytes/macrophages is in part dependent on ROCK.

The data (not shown) corroborate the fact that the compound of the invention has an in vitro cellular IC₅₀ which is similar or at least 10-20 fold more potent than prior art compounds.

eADME-tox Profile

The most relevant parameters for predicting human pharmacokinetics and toxicity have been established.

The compound of the invention shows improved and attractive eADME-tox properties (data not shown). Among these properties are: high solubility, medium to high permeability, required metabolic stability, no in vitro toxicity. These properties allow both systemic and topical application.

Compounds of the invention were also tested for potential effects on cell viability and cytotoxicity.

Cell viability and cytotoxicity were assessed in human embryonic kidney cells (HEK293T). HEK293T cells were incubated for 48 hours in the presence of compound. At the end of the incubation period, cell viability was assessed by quantification of the ATP content (CellTiter Glo, Promega) and the supernatant was assayed for presence of LDH (CytoTox One, Promega), both of which serve as a markers for cytotoxicity.

Up to concentrations of 30 μM, no effects on cell viability and cytotoxicity were observed for the compound of the invention.

### PK Profile

The pharmacokinetic parameters of compounds of the invention were studied in male CD-1 mice using cassette administration (Data not shown).

No adverse reactions were observed during administration of a compound of the invention.

The overall systemic exposure translated into very good absolute bioavailability. The systemic levels of the compounds are consistent with achieving pharmacological enzyme inhibition for several hours post oral administration.

### In Vivo Biomarker Assay

ROCK plays a role in LPS induced cytokine production and especially TNFα.

The in vivo effects of compounds according to the invention were investigated.

Male mice were dosed with 30 mg/kg of compound of the invention or vehicle via PO or IP routes. Following 2 h and 4 h post dose mice were challenged with LPS and a blood sample was taken 1 h after the LPS challenge to investigate the blood TNFα level using off the shelf technology.

The results (not shown) corroborate the fact that the compound of the invention is capable of suppressing the TNFα release efficiently after IP and PO dosing.

### Carrageenin Model of Inflammation

The efficiency of compounds of the invention to reduce acute inflammation was evaluated using the Carrageenin model: 5-6 weeks old Swiss Webster mice (Harlan) were weighed and the right paw volume measured by water displacement at the start of the experiment. The animals (n=10) were administered with either vehicle or 10 mg/kg 30 mg/kg of a compound of the invention orally, 2 h before carrageenin injection into the paw. Two hours after oral administration animals were anesthetized and injected with 50 μl (10 mg/ml) carrageenin in the sub plantar region of the paw. Two, four and six hours following the injection the paw volumes were measured.

A reduction in paw volume was seen with both 10 mg/kg and 30 mg/kg of a compound of the invention. The two hour value has a significance of >99% according to the T test.

The results (not shown) corroborate the fact that the compound of the invention is capable of suppressing the inflammatory response in the Carrageenin model with 10 mg/kg being a dose with maximal effect.

The data described above corroborate the fact that the compound of the present invention is capable of selectively and specifically inhibit ROCK with very favorable eADME, Tox and PK-properties.

In conclusion, the compounds of the invention are particularly suitable as medicament and for the treatment of:

VSMC hypercontraction including but not limited to cerebral vasospasm, coronary vasospasm, hypertension, pulmonary hypertension, and sudden death,
Other SMC disorders including but not limited to bronchial asthma and glaucoma,
Arteriosclerotic diseases including but not limited to angina, myocardial infraction, restenosis, stroke, hypertensive vascular disease, heart failure, cardiac allograft vasculopathy and vein graft disease,
Other disorder including but not limited to osteoporosis, erectile dysfunction and cancers (such as metastasis, cell migration for example), spinal-cord injury, stroke, HIV, inflammatory and demyelinising diseases, Alzheimer's disease, neuropathic pain, asthma, pre-term labor, renal disease.

The present invention encompasses compounds 1 to 172 and stereoisomers, tautomers, racemics or a pharmaceutically acceptable salt and/or solvate thereof.

The present invention also encompasses methods for assigning a function in inflammation to a ROCK inhibitor, comprising the steps of: providing a ROCK inhibitor, testing the activity of said inhibitor on at least one parameter selected from:

(i) a LPS induced TNF release,
(ii) a carrageenin induced edema model,
and determining from a positive outcome of said at least one parameter the use of the ROCK inhibitor for preventing, alleviating, treating a condition or a disease related to inflammation.

The present invention also encompasses a method for assigning a function in inflammation to a compound, comprising the steps of: providing a compound, testing the activity of said compound on an in vitro or in vivo ROCK inhibition assay and determining from a positive outcome of said inhibition assay the use of the compound for preventing, alleviating, treating a condition or a disease related to inflammation. The present invention preferably encompasses a method for assigning a function in inflammation to a compound, comprising the steps of: providing a compound, testing activity of said compound on ROCK.

All patents, patent applications, and published references cited herein are hereby incorporated by reference in their entirety. While this invention has been particularly shown and described with references to preferred embodiments, it will be understood by those skilled in the art that various changes in form and details may be made without departing from the scope of the invention encompassed by the claims.

1. A compound of Formula I or II or a stereoisomer, tautomer, racemic, salt, hydrate, or solvate thereof, 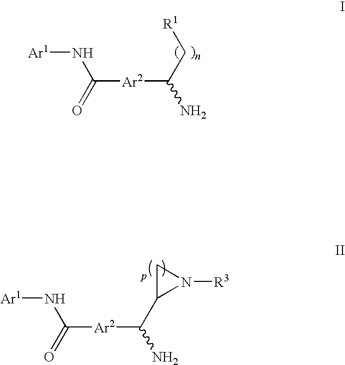
wherein:
Ar¹ is an aromatic 6-membered first ring containing carbon atoms and at least one nitrogen atom, said first ring being optionally fused to a saturated, unsaturated or aromatic 4-, 5-, 6-, or 7-membered second ring containing carbon atoms and optionally at least one nitrogen atom, said first or said second rings being independently substituted with one or more substituents independently selected from the group comprising hydrogen, halogen, alkyl, cycloalkyl, alkenyl, alkynyl, aralkyl, heteroarylalkyl, cycloalkylalkyl, acyl, aryl or heteroaryl wherein said substituents are optionally substituted by one or more further substituents selected from the group comprising halo, hydroxyl, oxo, nitro, amido, carboxy, amino, cyano, haloalkoxy, and haloalkyl;
Ar² is an aromatic 5- or 6-membered third ring containing carbon atoms and optionally one or two heteroatoms, said third ring optionally fused to an aromatic 6-membered fourth ring containing carbon atoms and optionally at least one heteroatom atom, wherein said third ring is optionally substituted with one or more substituents selected from the group comprising halogen, alkenyl, alkyl, alkynyl, acylamino, alkoxy, arylamino, nitro, haloalkoxy, aryl or heteroaryl, wherein said substituents are optionally substituted by one or more further substituents selected from the group comprising halo, hydroxyl, oxo, nitro, amido, carboxy, amino, cyano, haloalkoxy, and haloalkyl;
n is an integer selected from 0 or 1; and
p is an integer selected from 2, 3, 4 or 5; and
R¹ is selected from the Formula: 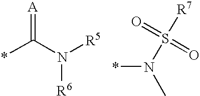
R³ is of Formula 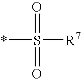
A is an oxygen or sulfur atom;
R⁵ is selected from hydrogen, alkyl, cycloalkyl, cycloalkylalkyl, aryl or aralkyl,
R⁶ and R⁷ are each independently selected from the group comprising:
hydrogen, a group selected from alkoxy, alkyl, alkylamino, alkylaminoalkyl, alkylcarbonyl, alkylcarbonylamino, amino, aralkyl, aryl, carbonylamino, cycloalkyl, formylamino, heteroaryl, heteroarylalkyl, heterocyclyl, or fused to the cycloalkyl, aryl, heterocyclyl or heteroaryl group may be one or more cycloalkyl, aryl, heterocyclyl or heteroaryl, or R⁵ and R⁶ together with the carbon atom to which they are attached form a heterocyclyl ring,
each group or said heterocyclyl ring being optionally substituted by one or more substituent selected from halo, alkenylaminooxy, alkoxy, alkyl, alkylamino, alkylaminosulfonyl, alkylcarbonyl, alkylcarbonylamino, alkyloxyaminoalkenyl, alkyloxycarbonyl, alkylsulfonyl, alkylsulfonylamino, alkylthio, amino, aralkyl, aryl, arylalkenylaminooxy, arylamino, arylaminosulfonyl, arylcarbonyl, arylcarbonylamino, aryloxy, cyano, cycloalkyl, haloalkoxy, haloalkyl, haloaryl, heteroaryl, heteroarylalkenylaminooxy, heteroarylalkyl, heteroarylcarbonylamino, heterocyclyl, hydroxyalkyl, nitro, oxo, sulfonyl, or fused to the cycloalkyl, aryl, heterocyclyl substituent or heteroaryl may be one or more cycloalkyl, aryl, heterocyclyl or heteroaryl,
each of said substituent being optionally substituted by one or more further substituent selected from halo, alkoxy, alkyl, alkylamino, alkylcarbonyl, alkylheteroaryl, alkylsuphonyl, aralkyl, aryl, arylamino, aryloxy, cyano, haloalkoxy, haloalkyl, heteroaryl, heteroarylalkyl, heteroarylcarbonyl, heterocyclyl, hydroxyl, nitro, oxo, or sulfonyl,
wherein when n is 1, R⁵ and R⁶ together with the carbon atom to which they are attached form a heterocyclyl or heteroaryl ring optionally substituted with one or more substituent selected from halo, alkenylaminooxy, alkoxy, alkyl, alkylamino, alkylaminosulfonyl, alkylcarbonyl, alkylcarbonylamino, alkyloxyaminoalkenyl, alkyloxycarbonyl, alkylsulfonyl, alkylsulfonylamino, alkylthio, amino, aralkyl, aryl, arylalkenylaminooxy, arylamino, arylaminosulfonyl, arylcarbonyl, arylcarbonylamino, aryloxy, cyano, cycloalkyl, haloalkoxy, haloalkyl, haloaryl, heteroaryl, heteroarylcarbonyl, heteroarylalkenylaminooxy, heteroarylalkyl, heteroarylcarbonylamino, heterocyclyl, hydroxyalkyl, nitro, oxo, sulfonyl, or fused to the cycloalkyl, aryl, heterocyclyl or heteroaryl substituent may be one or more cycloalkyl, aryl, heterocyclyl or heteroaryl,
or optionally fused with one or more aryl, heteroaryl, cycloalkyl, or heterocyclyl ring,
each of said substituent or fused ring being optionally substituted by one or more further substituent selected from halo, alkoxy, alkyl, alkylamino, alkylcarbonyl, alkyloxycarbonyl, alkylheteroaryl, alkylsuphonyl, aralkyl, aryl, arylamino, aryloxy, cyano, haloalkoxy, haloalkyl, heteroaryl, heteroarylalkyl, heteroarylcarbonyl, heterocyclyl, hydroxy, nitro, oxo, or sulfonyl.

2. The compound according to claim 1, having Formula III, IV, V or VI 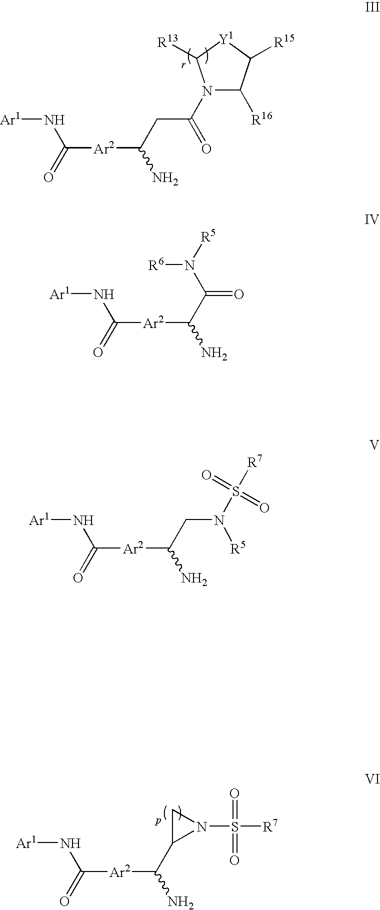
Y¹ is selected from —CH₂—, —CH(R¹⁴)—, —NH—, —O—, —S—, —C(═O)—,
p is selected from 2, 3 or 4,
r is an integer selected from 0, 1, 2 or 3,
wherein R¹³ and R¹⁴ are each independently selected from hydrogen or alkyl,
or R¹³ and R¹⁴ form together with the carbon atoms to which they are attached form an aryl, a heteroaryl, a cycloalkyl or a heterocyclyl
or r is 2 and two R¹³ form together with the carbon atoms to which they are attached form an aryl, an heteroaryl, a cycloalkyl or a heterocyclyl,
wherein R¹⁵ and R¹⁶ together with the carbon atom to which they are attached form an aryl, a cycloalkyl, a heteroaryl a heterocyclyl, each optionally substituted with one or more substituent selected from halo, alkoxy, alkyl, alkylamino, alkylcarbonyl, alkylheteroaryl, alkylsuphonyl, aralkyl, aryl, arylamino, aryloxy, cyano, haloalkoxy, haloalkyl, heteroaryl, heteroarylalkyl, heteroarylcarbonyl, heterocyclyl, hydroxy, nitro, oxo, or sulfonyl,
wherein Ar¹, Ar², R⁵, R⁶ and R⁷ have the same meaning as that defined in claim 1.

3. The compound according to claim 1, wherein Ar¹ is of the Formula: 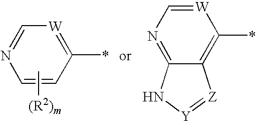
wherein
m is an integer selected from 0, 1, 2 or 3;
W is C(R²) or N;
Y and Z are independently selected from the group comprising N and CR²;
R² is selected from hydrogen, halogen, or a group selected from alkyl, cycloalkyl, alkenyl, alkynyl, aryl or heteroaryl wherein each of said group is optionally substituted by one or more substituents selected from the group comprising halo, hydroxyl, amido, carboxy, amino, cyano, haloalkoxy, and haloalkyl.

4. The compound according to claim 3, wherein W is N or C(R²), wherein R² has the same meaning as that defined in claim 3.

5. The compound according to claim 3, wherein W is CH or N.

6. The compound according to claim 3, wherein Y is CH and Z is CH or wherein Y is CH and Z is N, or wherein Y is N and Z is CH.

7. The compound according to claim 1, wherein Ar² is selected from 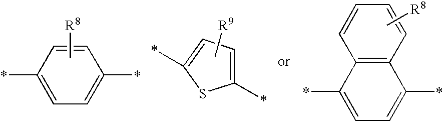
wherein R⁸ is selected from the group comprising hydrogen and halogen, alkenyl, alkyl, alkynyl, acylamino, alkoxy, arylamino, nitro, haloalkoxy, aryl or heteroaryl, optionally substituted by one or more substituents; and
R⁹ is selected from the group comprising hydrogen, halogen and alkyl.

8. The compound according to claim 1, having one of the structural Formula 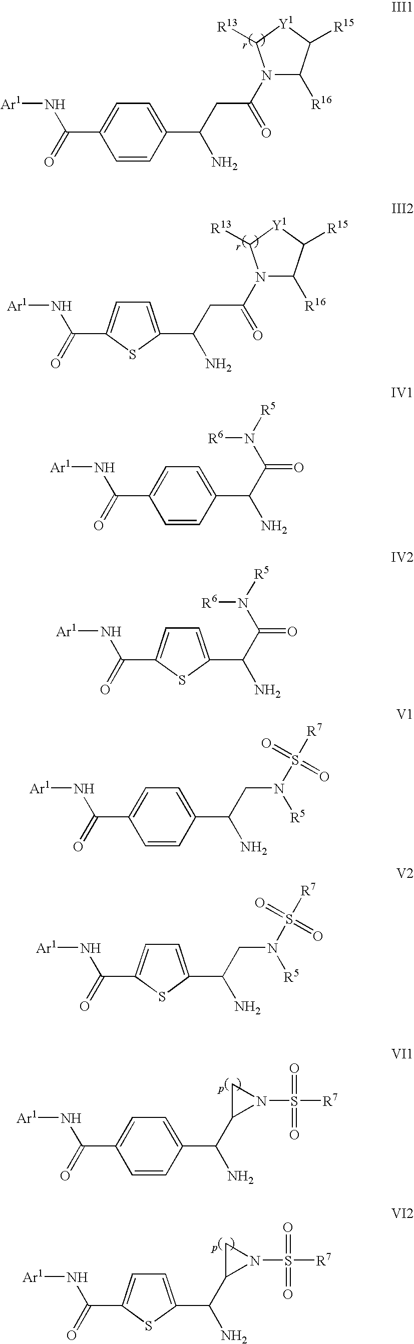
wherein Ar¹, R⁵, R⁶, R⁷, R¹³, R¹⁵, R¹⁶, r and p have the same meaning as that defined above.

9. The compound according to claim 1, wherein Ar¹ is heteroaryl and R⁵, R⁶ are each independently selected from optionally substituted aryl, alkyl, heteroaryl, aralkyl, heteroarylalkyl, fused arylcycloalkyl, cycloalkylalkyl, cycloalkyl, heterocyclyl, heterocyclylalkyl, alkoxyalkyl, alkylsulphonylaminoalkyl, alkoxy, hydroxyalkyl, alkylaminoalkyl, arylalkylheterocyloalkyl, alkylamino wherein the substituent is independently one or more optionally substituted alkyl, aryl, heteroaryl, cycloalkyl, heterocyclyl, amino, amido, oxo, nitro, carboxy, cyano, haloalkoxy, hydroxyl, halo, alkoxy, hydroxyalkyl, alkoxyalkoxy, alkoxyalkyl, aminoalkyl or alkylamino,
or wherein R⁵ and R⁶ together with the N to which they are attached form a optionally substituted heterocyclyl, or heteroaryl to which may be fused one or more optionally substituted aryl, cycloalkyl, heteroaryl, heterocyclyl, wherein the substituent is independently one or more alkyl, aryl, aralkyl, heteroaryl, cycloalkyl, heterocyclyl, amino, amido, oxo, nitro, carboxy, cyano, haloalkoxy, hydroxyl, halo, alkoxy, hydroxyalkyl, alkoxyalkoxy, alkoxyalkyl, aminoalkyl or alkylamino; each substituent being optionally substituted by one or more alkyl, aryl, halo, alkoxy, haloalkoxy, heteroaryl, heteroarylalkyl, aralkyl, hydroxyl, hydroxyalkyl.

10. The compound according to claim 1, wherein Ar¹ is heteroaryl and R⁷ is selected from optionally substituted alkyl, heteroaryl, cycloalkyl, heterocyclyl, or aryl to which may be fused one or more optionally substituted aryl, cycloalkyl, heteroaryl, heterocyclyl, wherein the substituent is independently one or more, alkyl, aryl, heteroaryl, cycloalkyl, heterocyclyl, amino, amido, oxo, nitro, carboxy, cyano, haloalkoxy, hydroxyl, halo, alkoxy, hydroxyalkyl, alkoxyalkoxy, alkoxyalkyl, aminoalkyl or alkylamino.

11. The compound according to claim 1, having one of the structural Formula, 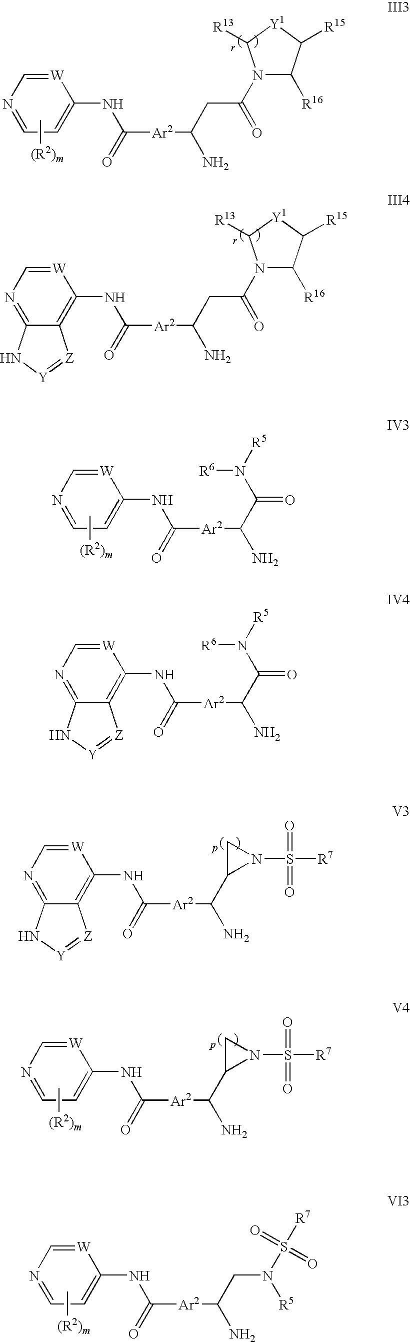 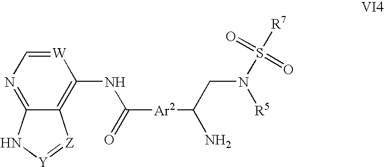
wherein Ar², W, Y, Z, Y¹, R², R⁵, R⁶, R⁷, R¹³, R¹⁵, R¹⁶, r, m and p have the same meaning as that defined above.

12. The compound according to claim 1, wherein Ar¹ is pyridin-4-yl.

13. The compound according to claim 1, having one of the structural Formula 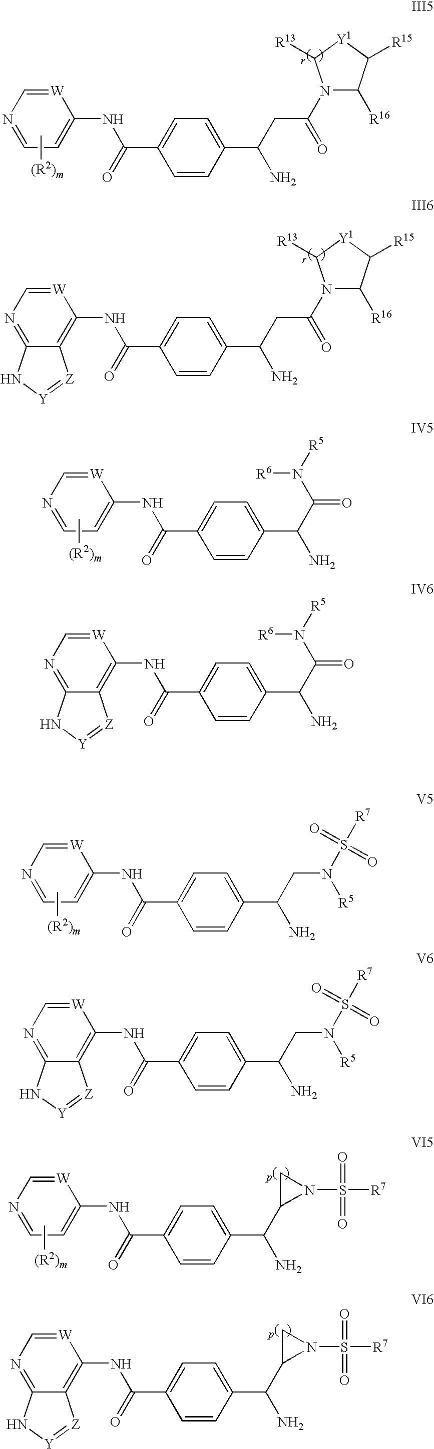
wherein W, Y, Z, Y¹, R², R⁵, R⁶, R⁷, R¹³, R¹⁵, R¹⁶, r, m and p have the same meaning as that defined above.

14. The compound according to claim 1,
wherein R⁵ is selected from hydrogen, alkyl or cycloalkyl, and
wherein W, Y, Z, Y¹, R², R⁶, R⁷, R¹³, R¹⁵, R¹⁶, r, m and p have the same meaning as that defined above.

15. The compound according to claim 1, wherein R⁶ is selected from alkyl, aryl, aralkyl, heteroaryl, heteroarylalkyl, cycloalkyl, cycloalkylalkyl, alkoxy, alkoxyalky, alkylsulphonylaminoalkyl, hydroxyalkyl, alkylaminoalkyl, heterocyclyl, heterocyclylalkyl, alkylamino, each being optionally substituted with one or more substituents selected from alkyl, alkoxy, halo, alkoxy, hydroxyl, hydroxyalkyl, aryl, heteroaryl or heterocyclyl, and
wherein W, Y, Z, Y¹, R², R⁵, R⁷, R¹³, R¹⁵, R¹⁶, r, m and p have the same meaning as that defined above.

16. The compound according to claim 1, wherein R⁵ and R⁶ together with the N to which they are attached form a optionally substituted heterocyclyl or heteroaryl to which may be fused one or more optionally substituted aryl, cycloalkyl, heteroaryl, heterocyclyl, wherein the substituent is independently selected from one or more alkyl, aryl, heteroaryl, aralkyl, cycloalkyl, heterocyclyl, amino, amido, oxo, nitro, carboxy, cyano, haloalkoxy, hydroxyl, halo, alkoxy, hydroxyalkyl, alkoxyalkoxy, alkoxyalkyl, aminoalkyl or alkylamino; each substituent being optionally substituted by one or more alkyl, aryl, halo, alkoxy, haloalkoxy, heteroaryl, heteroarylalkyl, aralkyl, hydroxyl, hydroxyalkyl, and
wherein W, Y, Z, Y¹, R², R⁷, R¹³, R¹⁵, R¹⁶, r, m and p have the same meaning as that defined above.

17. The compound according to claim 1, having one of the structural Formula 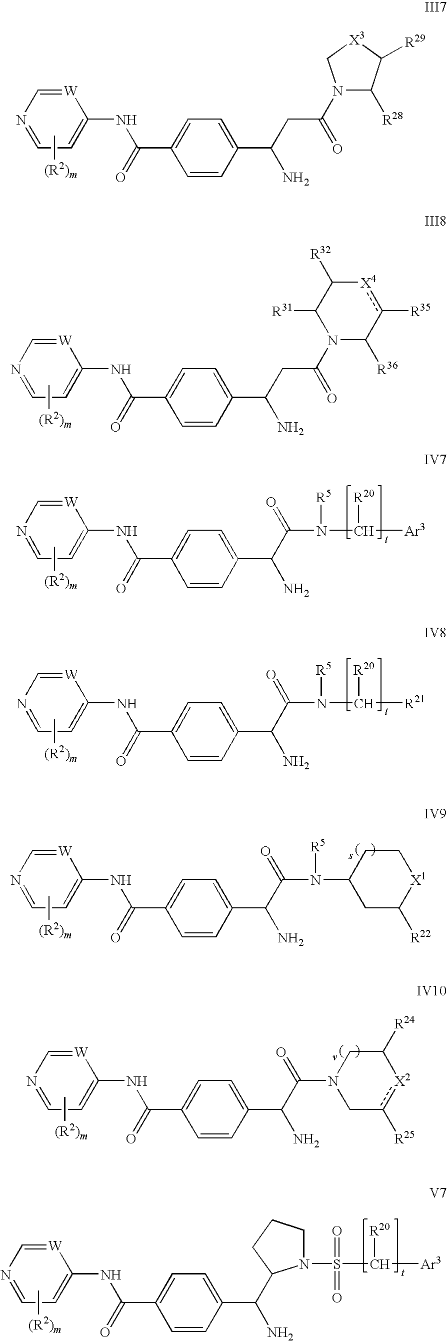 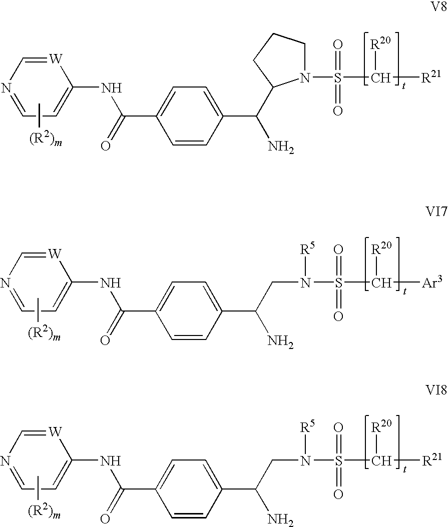
wherein
t is an integer selected from 0, 1, 2, 3, 4, 5, 6, 7 or 8,
s is an integer selected from 0 or 1,
v is an integer selected from 0 or 1,
R⁵ is selected from hydrogen, alkyl or cycloalkyl,
R²⁰ is selected from hydrogen or alkyl,
Ar³ is selected from aryl, heteroaryl, cycloalkyl, heterocyclyl, being each optionally substituted with one or more substituents selected from halo, alkoxy, alkyl, hydroxyalkyl, hydroxyl, aryl, aryloxy, aralkyl, heteroaryl, heteroarylalkyl,
R²¹ is selected from hydrogen, alkyl, cycloalkyl, alkoxy, alkylsulfonylamino, hydroxyl, alkylamino,
R²² is selected from hydrogen, alkyl, aryl,
X¹ is selected from CHR²³ or NR²³, wherein R²³ is selected from hydrogen, alkyl, aryl, aralkyl, heteroaryl, heteroarylalkyl, each being optionally substituted by one or more substituents selected from alkyl, aryl, aryloxy, aralkyl, halo, hydroxyl, alkoxy,
or wherein R²² and R²³ together with the carbon atoms to which they are attached form a optionally substituted aryl, heterocyclyl or heteroaryl, wherein the substituent is independently selected from one or more alkyl, aryl, heteroaryl, aralkyl, aryloxy, cycloalkyl, heterocyclyl, amino, amido, oxo, nitro, carboxy, cyano, haloalkoxy, hydroxyl, halo, alkoxy, hydroxyalkyl, alkoxyalkoxy, alkoxyalkyl, aminoalkyl or alkylamino;
X² is selected from O, NR²⁶ or C(R²⁶)R²⁷, wherein R²⁶ and R²⁷ are each independently hydrogen or are selected from the group consisting of alkyl, aryl, aralkyl, heteroaryl, hydroxyalkyl, alkoxy, hydroxyl, each group being optionally substituted by one or more alkyl, halo, aryl, aryloxy, aralkyl, alkoxy,
wherein R²⁴ and R²⁵ are each independently selected from hydrogen or are selected from the group consisting of alkyl, aryl, aralkyl, heteroaryl, hydroxyalkyl, alkoxy, hydroxyl, each group being optionally substituted by one or more alkyl, halo, aryl, aryloxy, aralkyl, alkoxy,
or wherein R²⁴ and R²⁶ together with the atoms to which they are attached form an optionally substituted aryl, heterocyclyl or heteroaryl, wherein the substituent is independently selected from one or more alkyl, aryl, heteroaryl, aralkyl, aryloxy, cycloalkyl, heterocyclyl, amino, amido, oxo, nitro, carboxy, cyano, haloalkoxy, hydroxyl, halo, alkoxy, hydroxyalkyl, alkoxyalkoxy, alkoxyalkyl, aminoalkyl or alkylamino;
X³ is selected from O, S, NR³⁰ or CHR³⁰ wherein R³⁰ is selected from hydrogen or a group selected from alkyl, hydroxyl, aryl, heteroaryl, hydroxyalkyl, heteroarylcarbonyl, aralkyl, each group being optionally substituted with one or more halo, alkyl, alkoxy, alkoxycarbonyl, aryl, heteroaryl,
wherein R²⁸ and R²⁹ are each independently selected from hydrogen or are selected from the group consisting of alkyl, hydroxyl, aryl, heteroaryl, hydroxyalkyl, heteroarylcarbonyl, aralkyl, each group being optionally substituted by one or more alkyl, halo, aryl, aryloxy, aralkyl, alkoxy,
or wherein R²⁹ and R³⁰ together with the atoms to which they are attached form an optionally substituted aryl, heterocyclyl or heteroaryl, wherein the substituent is independently selected from one or more alkyl, aryl, heteroaryl, aralkyl, aryloxy, cycloalkyl, heterocyclyl, amino, amido, oxo, nitro, carboxy, cyano, haloalkoxy, hydroxyl, halo, alkoxy, hydroxyalkyl, alkoxyalkoxy, alkoxyalkyl, aminoalkyl or alkylamino;
wherein X⁴ is selected from O, S, NR³³, C(R³³)R³⁴, wherein R³³ and R³⁴ are each independently hydrogen or selected from the group consisting of alkyl, hydroxyl, aryl, heteroaryl, hydroxyalkyl, heteroarylcarbonyl, aralkyl, each group being optionally substituted with one or more halo, alkyl, alkoxy, alkoxycarbonyl, aryl, heteroaryl,
wherein R³¹, R³², R³⁵ and R³⁶ are each independently selected from hydrogen or are selected from the group consisting of alkyl, hydroxyl, aryl, heteroaryl, hydroxyalkyl, heteroarylcarbonyl, aralkyl, each group being optionally substituted by one or more alkyl, halo, aryl, aryloxy, aralkyl, alkoxy,
or wherein R³¹ and R³² together with the atoms to which they are attached form an optionally substituted aryl, heterocyclyl or heteroaryl, wherein the substituent is independently selected from one or more alkyl, aryl, heteroaryl, aralkyl, aryloxy, cycloalkyl, heterocyclyl, amino, amido, oxo, nitro, carboxy, cyano, haloalkoxy, hydroxyl, halo, alkoxy, hydroxyalkyl, alkoxyalkoxy, alkoxyalkyl, aminoalkyl or alkylamino;
or wherein R³² and R³³ together with the atoms to which they are attached form an optionally substituted aryl, heterocyclyl or heteroaryl, wherein the substituent is independently selected from one or more alkyl, aryl, heteroaryl, aralkyl, aryloxy, cycloalkyl, heterocyclyl, amino, amido, oxo, nitro, carboxy, cyano, haloalkoxy, hydroxyl, halo, alkoxy, hydroxyalkyl, alkoxyalkoxy, alkoxyalkyl, aminoalkyl or alkylamino,
and wherein the dotted line represents an optional double bond.

18. The compound according to claim 1, selected from 4-(amino-benzylcarbamoyl)-methyl)-N-pyridin-4-yl-benzamide; 4-[amino-(1-phenyl-ethylcarbamoyl)-methyl]-N-pyridin-4-yl-benzamide; 4-[amino-(benzyl-methyl-carbamoyl)-methyl]-N-pyridin-4-yl-benzamide; 4-[amino-(1-phenyl-propylcarbamoyl)-methyl]-N-pyridin-4-yl-benzamide; 4-[amino-(1-(S)-phenyl-propylcarbamoyl)-methyl]-N-pyridin-4-yl-benzamide; 4-[amino-(2-chloro-benzylcarbamoyl)-methyl]-N-pyridin-4-yl-benzamide; 4-[amino-(3-chloro-benzylcarbamoyl)-methyl]-N-pyridin-4-yl-benzamide; 4-[amino-(4-methoxy-benzylcarbamoyl)-methyl]-N-pyridin-4-yl-benzamide; 4-[amino-(2-methoxy-benzylcarbamoyl)-methyl]-N-pyridin-4-yl-benzamide; 4-{amino-[(pyridin-2-ylmethyl)-carbamoyl]-methyl}-N-pyridin-4-yl-benzamide; 4-{amino-[(pyridin-4-ylmethyl)-carbamoyl]-methyl}-N-pyridin-4-yl-benzamide; 4-{amino-[(furan-2-ylmethyl)-carbamoyl]-methyl}-N-pyridin-4-yl-benzamide; 4-[1-amino-2-(3,4-dihydro-1H-isoquinolin-2-yl)-2-oxo-ethyl]-N-pyridin-4-yl-benzamide; 4-(amino-phenethylcarbamoyl-methyl)-N-pyridin-4-yl-benzamide; 4-[amino-(indan-2-ylcarbamoyl)-methyl]-N-pyridin-4-yl-benzamide; 4-{amino-[2-(4-fluoro-phenyl)ethylcarbamoyl]-methyl}-N-pyridin-4-yl-benzamide; 4-[amino-(2-thiophen-2-yl-ethylcarbamoyl)-methyl]-N-pyridin-4-yl-benzamide; 4-[amino-(2-pyrazol-1-yl-ethylcarbamoyl)-methyl]-N-pyridin-4-yl-benzamide; 4-[amino-(2-imidazol-1-yl-ethylcarbamoyl)-methyl]-N-pyridin-4-yl-benzamide; 4-(amino-phenylcarbamoyl-methyl)-N-pyridin-4-yl-benzamide; 4-[amino-(4-methoxy-phenylcarbamoyl)-methyl]-N-pyridin-4-yl-benzamide; 4-(amino-ethylcarbamoyl-methyl)-N-pyridin-4-yl-benzamide; 4-[amino-(1,2-dimethyl-propylcarbamoyl)-methyl]-N-pyridin-4-yl-benzamide; 4-[amino-(1,1-dimethyl-propylcarbamoyl)-methyl]-N-pyridin-4-yl-benzamide; 4-[amino-(cyclopropylmethyl-carbamoyl)-methyl]-N-pyridin-4-yl-benzamide; 4-[1-amino-2-(1-methyl-piperidin-1-yl)-2-oxo-ethyl]-N-pyridin-4-yl-benzamide; 4-[1-amino-2-(3,5-dimethyl-piperidin-1-yl)-2-oxo-ethyl]-N-pyridin-4-yl-benzamide; 4-[1-amino-2-(4-phenyl-piperidin-1-yl)-2-oxo-ethyl]-N-pyridin-4-yl-benzamide; 4-{1-amino-2-[4-(4-fluoro-phenyl)-3,6-dihydro-2H-pyridin-1-yl]-2-oxo-ethyl}-N-pyridin-4-yl-benzamide; 4-[1-amino-2-(4-benzooxazol-2-yl-piperidin-1-yl)-2-oxo-ethyl]-N-pyridin-4-yl-benzamide; 4-[1-amino-2-(4-benzyl-piperidin-1-yl)-2-oxo-ethyl]-N-pyridin-4-yl-benzamide; 4-(amino-cyclohexylcarbamoyl-methyl)-N-pyridin-4-yl-benzamide; 4-[amino-(4-methyl-cyclohexylcarbamoyl)-methyl]-N-pyridin-4-yl-benzamide; 4-(1-amino-2-mopholin-4-yl)-2-oxo-ethyl)-N-pyridin-4-yl-benzamide; 4-[1-amino-2-(2,6-dimethyl-mopholin-4-yl)-2-oxo-ethyl]-N-pyridin-4-yl-benzamide; 4-[1-amino-2-(3-hydroxymethyl-piperidin-1-yl)-2-oxo-ethyl]-N-pyridin-4-yl-benzamide; 4-{amino-[(tetrahydro-furan-2-ylmethyl)-carbamoyl]-methyl}-N-pyridin-4-yl-benzamide; 4-[1-amino-2-(4-hydroxy-4-phenyl-piperidin-1-yl)-2-oxo-ethyl]-N-pyridin-4-yl-benzamide; 4-[amino-(2-methoxy-ethylcarbamoyl)-methyl]-N-pyridin-4-yl-benzamide; 4-[amino-(2-methanesulfonylamino-ethylcarbamoyl)-methyl]-N-pyridin-4-yl-benzamide; 4-(amino-methoxycarbamoyl-methyl)-N-pyridin-4-yl-benzamide; 4-{amino-[(2-hydroxy-ethyl)-methyl-carbamoyl]-methyl}-N-pyridin-4-yl-benzamide; 4-[amino-(2-methoxy-1-methyl-ethylcarbamoyl)-methyl]-N-pyridin-4-yl-benzamide; 4-[amino-(2-dimethylamino-ethylcarbamoyl)-methyl]-N-pyridin-4-yl-benzamide; 4-[amino-(2-morpholin-4-yl-ethylcarbamoyl)-methyl]-N-pyridin-4-yl-benzamide; 4-[1-amino-2-(4-isopropyl-piperazin-1-yl)-2-oxo-ethyl]-N-pyridin-4-yl-benzamide; 4-[1-amino-2-oxo-2-(4-phenyl-piperzin-1-yl)-ethyl]-N-pyridin-4-yl-benzamide; 4-{1-amino-2-[4-(2-fluoro-benzyl)-piperazin-1-yl]-2-oxo-ethyl}-N-pyridin-4-yl-benzamide; 4-{1-amino-2-[4-(4-fluoro-benzyl)-piperazin-1-yl]-2-oxo-ethyl}-N-pyridin-4-yl-benzamide; 4-{1-amino-2-[4-(4-methoxy-benzyl)-piperazin-1-yl]-2-oxo-ethyl}-N-pyridin-4-yl-benzamide; 4-{amino-[1-benzyl-piperidin-4-ylcarbamoyl]-methyl}-N-pyridin-4-yl-benzamide; 4-{1-amino-2-[4-(2-hydroxy-ethyl)-piperazin-1-yl]-2-oxo-ethyl-N-pyridin-4-yl-benzamide; 4-{amino-[1-(5-methyl-thiophen-2-ylmethyl)-piperidin-4-ylcarbamoyl]-methyl}-N-pyridin-4-yl-benzamide; 4-[amino-(N′-butyl-hydrazinocarbonyl)-methyl]-N-pyridin-4-yl-benzamide; 4-{1-amino-2-[2-(3-fluoro-phenyl)-pyrrolidin-1-yl]-2-oxo-ethyl}-N-pyridin-4-yl-benzamide; 4-(amino-benzylcarbamoyl-methyl)-N-(1H-pyrrolo[2,3-b]pyridin-4-yl)-benzamide; 4-(amino-benzylcarbamoyl-methyl)-N-(1H-pyrazolo[3,4-b]pyridin-4-yl)-benzamide; 4-(amino-benzylcarbamoyl-methyl)-N-(9H-purin-6-yl)-benzamide; 4-[amino-(3-chloro-benzylcarbamoyl)-methyl]-N-(1H-pyrrolo[2,3-b]pyridin-4-yl)-benzamide; 4-[amino-(3-chloro-benzylcarbamoyl)-methyl]-N-(1H-pyrazolo[3,4-b]pyridin-4-yl)-benzamide; 4-[amino-(3-chloro-benzylcarbamoyl)-methyl]-N-(9H-purin-6-yl)-benzamide; 4-((S)-amino-benzylcarbamoyl-methyl)-N-pyridin-4-yl-benzamide; or 4-((R)-amino-benzylcarbamoyl-methyl)-N-pyridin-4-yl-benzamide.

19. The compound according to claim 1, selected from 4-(1-amino-2-methanesulfonylamino-ethyl)-N-pyridin-4-yl-benzamide; 4-(1-amino-2-ethanesulfonylamino)-ethyl)-N-pyridin-4-yl-benzamide; 4-[1-amino-2-(propane-1-sulfonylamino)-ethyl]-N-pyridin-4-yl-benzamide; 4-[1-amino-2-(butane-1-sulfonylamino)-ethyl]-N-pyridin-4-yl-benzamide; 4-[1-amino-2-(octane-1-sulfonylamino)-ethyl]-N-pyridin-4-yl-benzamide; 4-(1-amino-2-phenylmethanesulfonylamino-ethyl)-N-pyridin-4-yl-benzamide; 4-(1-amino-2-benzenesulfonylamino-ethyl)-N-pyridin-4-yl-benzamide; 4-[1-amino-2-(4-methyl-benzenesulfonylamino)-ethyl]-N-pyridin-4-yl-benzamide; 4-[1-amino-2-(3-methyl-benzenesulfonylamino)-ethyl]-N-pyridin-4-yl-benzamide; 4-[1-amino-2-(2-methyl-benzenesulfonylamino)-ethyl]-N-pyridin-4-yl-benzamide; 4-[1-amino-2-(4-fluoro-benzenesulfonylamino)-ethyl]-N-pyridin-4-yl-benzamide; 4-[1-amino-2-(2-fluoro-benzenesulfonylamino)-ethyl]-N-pyridin-4-yl-benzamide; 4-[1-amino-2-(4-chloro-benzenesulfonylamino)-ethyl]-N-pyridin-4-yl-benzamide; 4-[1-amino-2-(3-chloro-benzenesulfonylamino)-ethyl]-N-pyridin-4-yl-benzamide; 4-[1-amino-2-(2-chloro-benzenesulfonylamino)-ethyl]-N-pyridin-4-yl-benzamide; 4-[1-amino-2-(4-methoxy-benzenesulfonylamino)-ethyl]-N-pyridin-4-yl-benzamide; 4-[1-amino-2-(3,4-dimethoxy-benzenesulfonylamino)-ethyl]-N-pyridin-4-yl-benzamide; 4-[1-amino-2-(2,5-dimethoxy-benzenesulfonylamino)-ethyl]-N-pyridin-4-yl-benzamide; 4-[1-amino-2-(3-phenoxy-benzenesulfonylamino)-ethyl]-N-pyridin-4-yl-benzamide; 4-[1-amino-2-(4-phenoxy-benzenesulfonylamino)-ethyl]-N-pyridin-4-yl-benzamide; 4-[1-amino-2-(biphenyl-3-sulfonylamino-ethyl)-N-pyridin-4-yl-benzamide; 4-[1-amino-2-(biphenyl-4-sulfonylamino-ethyl)-N-pyridin-4-yl-benzamide; 4-[1-amino-2-(naphthalene-1-sulfonylamino)-ethyl]-N-pyridin-4-yl-benzamide; 4-[1-amino-2-(thiophene-2-sulfonylamino)-ethyl]-N-pyridin-4-yl-benzamide; 4-[1-amino-2-(3,5-dimethyl-isoxazole-4-sulfonylamino)-ethyl]-N-pyridin-4-yl-benzamide; 4-[1-amino-2-(5-chloro-1,3-dimethyl-1H-pyrazole-4-sulfonylamino)-ethyl]-N-pyridin-4-yl-benzamide; 4-[1-amino-2-(benzo[1,2,5]-oxadiazole-4-sulfonylamino)-ethyl]-N-pyridin-4-yl-benzamide; 4-[1-amino-2-(benzo[1,2,5]-thiadiazole-4-sulfonylamino)-ethyl]-N-pyridin-4-yl-benzamide; 4-[1-amino-2-(4-methoxy-benzenesulfonylamino)-ethyl]-N-(1H-pyrrolo[2,3-b]pyridin-4-yl)-benzamide; 4-[1-amino-2-(4-methoxy-benzenesulfonylamino)-ethyl]-N-(1H-pyrazolo[3,4-b]pyridin-4-yl)-benzamide; 4-[1-amino-2-(4-methoxy-benzenesulfonylamino)-ethyl]-N-(9H-purin-6-yl)-benzamide; 4-[1-amino-2-(benzenesulfonyl-methyl-amino)-ethyl]-N-pyridin-4-yl-benzamide; 4-{1-amino-2-[(4-fluoro-benzenesulfonyl)-methyl-amino]-ethyl-N-pyridin-4-yl-benzamide; 4-{1-amino-2-[(3-fluoro-benzenesulfonyl)-methyl-amino]-ethyl}-N-pyridin-4-yl-benzamide; 4-{1-amino-2-[(2-fluoro-benzenesulfonyl)-methyl-amino]-ethyl}-N-pyridin-4-yl-benzamide; 4-{1-amino-2-[(2-chloro-benzenesulfonyl)-methyl-amino]-ethyl}-N-pyridin-4-yl-benzamide dihydrochloric acid salt; 4-{1-amino-2-[(3-chloro-benzenesulfonyl)-methyl-amino]-ethyl}-N-pyridin-4-yl-benzamide; 4-{1-amino-2-[(4-chloro-benzenesulfonyl)-methyl-amino]-ethyl}-N-pyridin-4-yl-benzamide; 4-{1-amino-2-[(4-methoxy-benzenesulfonyl)-methyl-amino]-ethyl}-N-pyridin-4-yl-benzamide; 4-(R)1-amino-2-[(4-methoxy-benzenesulfonyl)-methyl-amino]-ethyl}-N-pyridin-4-yl-benzamide; or 4-{(S)1-amino-2-[(4-methoxy-benzenesulfonyl)-methyl-amino]-ethyl}-N-pyridin-4-yl-benzamide.

20. The compound according to claim 1, selected from 4-[1-amino-3-oxo-3-(2-phenyl-pyrrolidin-1-yl)-propyl]-pyridin-4-yl-benzamide; 4-{1-amino-3-[2-(3-chloro-phenyl)-pyrrolidin-1-yl]-3-oxo-propyl}-N-pyridin-4-yl-benzamide; 4-{1-amino-3-[2-(4-fluoro-phenyl)-pyrrolidin-1-yl]-3-oxo-propyl}-N-pyridin-4-yl-benzamide; 4-{1-amino-3-[2-(3-fluoro-phenyl)-pyrrolidin-1-yl]-3-oxo-propyl}-N-pyridin-4-yl-benzamide; 4-{1-amino-3-[2-(2,5-difluoro-phenyl)-pyrrolidin-1-yl]-3-oxo-propyl}-N-pyridin-4-yl-benzamide; 4-{1-amino-3-[2-(2,4-difluoro-phenyl)-pyrrolidin-1-yl]-3-oxo-propyl}-N-pyridin-4-yl-benzamide; 4-{1-amino-3-[2-(4-chloro-phenyl)-pyrrolidin-1-yl]-3-oxo-propyl}-N-pyridin-4-yl-benzamide; 4-{1-amino-3-[2-(3,4-dichloro-phenyl)-pyrrolidin-1-yl]-3-oxo-propyl}-N-pyridin-4-yl-benzamide; 4-[1-amino-3-oxo-3-(2-phenyl-piperidin-1-yl)-propyl]-N-pyridin-4-yl-benzamide; 4-[1-amino-3-(2-naphthalen-1-yl-piperidin-1-yl)-3-oxo-propyl]-N-pyridin-4-yl-benzamide; 4-{1-amino-3-[2-(4-chloro-phenyl)-piperidin-1-yl]-3-oxo-propyl}-N-pyridin-4-yl-benzamide; 4-{1-amino-3-[2-(2,5-dichloro-phenyl)-piperidin-1-yl]-3-oxo-propyl}-N-pyridin-4-yl-benzamide; 4-{1-amino-3-[2-(5-chloro-2-methoxy-phenyl)-piperidin-1-yl]-3-oxo-propyl}-N-pyridin-4-yl-benzamide; 4-{1-amino-3-[2-(4-fluoro-phenyl)-piperidin-1-yl]-3-oxo-propyl}-N-pyridin-4-yl-benzamide; 4-{1-amino-3-[2-(3-methoxy-phenyl)-piperidin-1-yl]-3-oxo-propyl}-N-pyridin-4-yl-benzamide; 4-[1-amino-3-oxo-3-(3-phenyl-morpholin-4-yl)-propyl]-N-pyridin-4-yl-benzamide; 4-{1-amino-3-[3-(4-chloro-phenyl)-morpholin-4-yl]-3-oxo-propyl}-N-pyridin-4-yl-benzamide; 4-{1-amino-3-[3-(4-fluoro-phenyl)-morpholin-4-yl]-3-oxo-propyl}-N-pyridin-4-yl-benzamide; 4-[1-amino-3-(5-chloro-3,4-dihydro-1H-isoquinolin-2-yl)-3-oxo-propyl]-N-pyridin-4-yl-benzamide; 4-[1-amino-3-(7-chloro-3,4-dihydro-1H-isoquinolin-2-yl)-3-oxo-propyl]-N-pyridin-4-yl-benzamide; 4-[1-amino-3-(7-fluoro-3,4-dihydro-1H-isoquinolin-2-yl)-3-oxo-propyl]-N-pyridin-4-yl-benzamide; 4-[1-amino-3-(1,3-dihydro-isoindol-2-yl)-3-oxo-propyl]-N-pyridin-4-yl-benzamide; 4-(1-amino-3-oxo-3-thiazolidin-3-yl-propyl)-N-pyridin-4-yl-benzamide; 4-(1-amino-3-oxo-3-piperidin-1-yl-propyl)-N-pyridin-4-yl-benzamide; 4-[1-amino-3-(3,5-dimethyl-piperidin-1-yl)-3-oxo-propyl]-N-pyridin-4-yl-benzamide; 4-[1-amino-3-(3-hydroxymethyl-piperidin-1-yl)-3-oxo-propyl]-N-pyridin-4-yl-benzamide; 4-[1-amino-3-(octahydro-quinolin-1-yl)-3-oxo-propyl]-N-pyridin-4-yl-benzamide; 4-[1-amino-3-(4-hydroxy-4-phenyl-piperidin-1-yl)-3-oxo-propyl]-N-pyridin-4-yl-benzamide; 4-(1-amino-3-morpholin-4-yl-3-oxo-propyl)-N-pyridin-4-yl-benzamide; 4-[1-amino-3-(2,6-dimethyl-morpholin-4-yl)-3-oxo-propyl]-N-pyridin-4-yl-benzamide; 4-[1-amino-3-(4-isopropyl-piperazin-1-yl)-3-oxo-propyl]-N-pyridin-4-yl-benzamide; 4-{1-amino-3-[4-(2-hydroxy-ethyl)-piperazin-1-yl]-3-oxo-propyl}-N-pyridin-4-yl-benzamide; 4-[1-amino-3-oxo-3-(4-phenyl-piperazin-1-yl)-propyl]-N-pyridin-4-yl-benzamide; 4-[1-amino-3-(4-phenyl-piperidin-1-yl)-3-oxo-propyl]-N-pyridin-4-yl-benzamide; 4-{1-amino-3[4-(4-fluoro-phenyl)-3,6-dihydro-2H-pyridin-1-yl]-3-oxo-propyl}-N-pyridin-4-yl-benzamide; 4-[1-amino-3-(4-benzyl-piperidin-1-yl)-3-oxo-propyl]-N-pyridin-4-yl-benzamide; 4-{1-amino-3-(4-benzooxazol-2-yl-piperidin-1-yl)-3-oxo-propyl}-N-pyridin-4-yl-benzamide; 4-{1-Amino-3-[4-(4-methoxy-benzyl)-piperazin-1-yl]-3-oxo-propyl}-N-pyridin-4-yl-benzamide; 4-{1-amino-3-[4-(2-fluoro-benzyl)-piperazin-1-yl]-3-oxo-propyl}-N-pyridin-4-yl-benzamide; 4-{1-amino-3-[4-(4-fluoro-benzyl)-piperazin-1-yl]-3-oxo-propyl}-N-pyridin-4-yl-benzamide; 4-{1-amino-3-[4-(furan-2-carbonyl)-piperazin-1-yl]-3-oxo-propyl}-N-pyridin-4-yl-benzamide; 4-{1-amino-3-[4-(1H-indol-3-yl)-piperidin-1-yl]-3-oxo-propyl}-N-pyridin-4-yl-benzamide; 2-{3-Amino-3-[4-(pyridin-4-ylcarbamoyl)-phenyl]-propionyl}-1,2,3,4-tetrahydro-isoquinoline-6-carboxylic acid methyl ester; 4(1-{3-Amino-3-[4-(pyridin-4-ylcarbamoyl)-phenyl]-propionyl}-piperidin-2-yl)-benzoic acid methyl ester; 4-[1-amino-3-oxo-(1,3,4,9-tetrahydro-β-carbolin-2-yl)-propyl]-N-pyridin-4-yl-benzamide; 4-{1-amino-3-[1-(3-chloro-phenyl)-1,3,4,9-tetrahydro-β-carbolin-2-yl]-3-oxo-propyl}-N-pyridin-4-yl-benzamide; 4-{1-amino-3-[1-(3-bromo-4-methoxy-phenyl)-1,3,4,9-tetrahydro-β-carbolin-2-yl]-3-oxo-propyl}-N-pyridin-4-yl-benzamide; 4-[1-amino-3-(3,4-dihydro-1H-isoquinolin-2-yl)-3-oxo-propyl]-N-pyridin-4-yl-benzamide; 4-{1-amino-3-[2-(3-fluoro-phenyl)-pyrrolidin-1-yl]-3-oxo-propyl}-N-(1H-pyrrolo[2,3-b]pyridin-4-yl)-benzamide; 4-{1-amino-3-[2-(3-fluoro-phenyl)-pyrrolidin-1-yl]-3-oxo-propyl}-N-(1H-pyrazolo[3,4-b]pyridin-4-yl)-benzamide; 4-{1-amino-3-[2-(3-fluoro-phenyl)-pyrrolidin-1-yl]-3-oxo-propyl}-N-(9H-purin-6-yl)-benzamide; 4-[(R)-1-amino-3-(7-fluoro-3,4-dihydro-1H-isoquinolin-2-yl)-3-oxo-propyl]-N-pyridin-4-yl-benzamide; 4-[(S)-1-amino-3-(7-fluoro-3,4-dihydro-1H-isoquinolin-2-yl)-3-oxo-propyl]-N-pyridin-4-yl-benzamide; 4-{(R)-1-amino-3-[(R)-2-(3-fluoro-phenyl)-pyrrolidin-1-yl]-3-oxo-propyl}-N-pyridin-4-yl-benzamide; 4-{(R)-1-amino-3-[(S)-2-(3-fluoro-phenyl)-pyrrolidin-1-yl]-3-oxo-propyl}-N-pyridin-4-yl-benzamide; 4-{(S)-1-amino-3-[(R)-2-(3-fluoro-phenyl)-pyrrolidin-1-yl]-3-oxo-propyl}-N-pyridin-4-yl-benzamide; or 4-{(S)-1-amino-3-[(S)-2-(3-fluoro-phenyl)-pyrrolidin-1-yl]-3-oxo-propyl}-N-pyridin-4-yl-benzamide.

21. The compound according to claim 1, selected from 4-{amino-[1-(4-methoxy-benzenesulfonyl)-pyrrolidin-2-yl]-methyl}-N-pyridin-4-yl-benzamide; 4-{amino-[1-(2-chloro-benzenesulfonyl)-pyrrolidin-2-yl]-methyl}-N-pyridin-4-yl-benzamide; 4-{amino-[1-(3-chloro-benzenesulfonyl)-pyrrolidin-2-yl]-methyl}-N-pyridin-4-yl-benzamide; 4-{amino-[1-(4-chloro-benzenesulfonyl)-pyrrolidin-2-yl]-methyl}-N-pyridin-4-yl-benzamide; 4-{amino-[1-(2-fluoro-benzenesulfonyl)-pyrrolidin-2-yl]-methyl}-N-pyridin-4-yl-benzamide; 4-{amino-[1-(3-fluoro-benzenesulfonyl)-pyrrolidin-2-yl]-methyl}-N-pyridin-4-yl-benzamide; or 4-{amino-[1-(4-fluoro-benzenesulfonyl)-pyrrolidin-2-yl]-methyl}-N-pyridin-4-yl-benzamide.

22. The compound according to claim 1, selected from 4-(amino-benzylcarbamoyl)-methyl)-N-pyridin-4-yl-benzamide; 4-[amino-(2-chloro-benzylcarbamoyl)-methyl]-N-pyridin-4-yl-benzamide; 4-[amino-(3-chloro-benzylcarbamoyl)-methyl]-N-pyridin-4-yl-benzamide; 4-{1-amino-2-[2-(3-fluoro-phenyl)-pyrrolidin-1-yl]-2-oxo-ethyl}-N-pyridin-4-yl-benzamide; 4-((S)-amino-benzylcarbamoyl-methyl)-N-pyridin-4-yl-benzamide.

23. The compound according to claim 1, selected from 4-[1-amino-2-(4-methyl-benzenesulfonylamino)-ethyl]-N-pyridin-4-yl-benzamide; 4-[1-amino-2-(4-chloro-benzenesulfonylamino)-ethyl]-N-pyridin-4-yl-benzamide; 4-[1-amino-2-(4-methoxy-benzenesulfonylamino)-ethyl]-N-pyridin-4-yl-benzamide; 4-{1-amino-2-[(4-methoxy-benzenesulfonyl)-methyl-amino]-ethyl}-N-pyridin-4-yl-benzamide.

24. The compound according to claim 1, selected from 4-[1-amino-3-oxo-3-(2-phenyl-pyrrolidin-1-yl)-propyl]-N-pyridin-4-yl-benzamide; 4-{1-amino-3-[2-(3-chloro-phenyl)-pyrrolidin-1-yl]-3-oxo-propyl}-N-pyridin-4-yl-benzamide; 4-{1-amino-3-[2-(4-fluoro-phenyl)-pyrrolidin-1-yl]-3-oxo-propyl}-N-pyridin-4-yl-benzamide; 4-{1-amino-3-[2-(3-fluoro-phenyl)-pyrrolidin-1-yl]-3-oxo-propyl}-N-pyridin-4-yl-benzamide; 4-{1-amino-3-[2-(2,4-difluoro-phenyl)-pyrrolidin-1-yl]-3-oxo-propyl}-N-pyridin-4-yl-benzamide; 4-{1-amino-3-[2-(4-chloro-phenyl)-pyrrolidin-1-yl]-3-oxo-propyl}-N-pyridin-4-yl-benzamide; 4-{1-amino-3-[2-[3,4-dichloro-phenyl)-pyrrolidin-1-yl]-3-oxo-propyl}-N-pyridin-4-yl-benzamide; 4-[1-Amino-3-oxo-3-(2-phenyl-piperidin-1-yl)-propyl]-N-pyridin-4-yl-benzamide; 4-[1-amino-3-(2-naphthalen-1-yl-piperidin-1-yl)-3-oxo-propyl]-N-pyridin-4-yl-benzamide; 4-{1-amino-3-[2-(4-chloro-phenyl)-piperidin-1-yl]-3-oxo-propyl}-N-pyridin-4-yl-benzamide; 4-{1-amino-3-[2-(3-methoxy-phenyl)-piperidin-1-yl]-3-oxo-propyl}-N-pyridin-4-yl-benzamide; 4-{1-amino-3-[3-(4-chloro-phenyl)-morpholin-4-yl]-3-oxo-propyl}-N-pyridin-4-yl-benzamide; 4-{1-amino-3-[3-(4-fluoro-phenyl)-morpholin-4-yl]-3-oxo-propyl]-N-pyridin-4-yl-benzamide; 4-[1-amino-3-(7-chloro-3,4-dihydro-1H-isoquinolin-2-yl)-3-oxo-propyl]-N-pyridin-4-yl-benzamide; 4-[(S)-1-amino-3-(7-fluoro-3,4-dihydro-1H-isoquinolin-2-yl)-3-oxo-propyl]-N-pyridin-4-yl-benzamide; 4-{(R)-1-amino-3-[(R)-2-(3-fluoro-phenyl)-pyrrolidin-1-yl]-3-oxo-propyl}-N-pyridin-4-yl-benzamide; 4-{(R)-1-amino-3-[(S)-2-(3-fluoro-phenyl)-pyrrolidin-1-yl]-3-oxo-propyl}-N-pyridin-4-yl-benzamide; 4-{(S)-1-amino-3-[(R)-2-(3-fluoro-phenyl)-pyrrolidin-1-yl]-3-oxo-propyl}-N-pyridin-4-yl-benzamide; 4-{(S)-1-amino-3-[(S)-2-(3-fluoro-phenyl)-pyrrolidin-1-yl]-3-oxo-propyl}-N-pyridin-4-yl-benzamide.

25. A pharmaceutical and/or veterinary composition comprising a compound as defined in claim 1.

26. A pharmaceutical and/or veterinary composition comprising at least one compound according to claim 1 and at least one carrier, excipient or diluent acceptable for pharmaceutical and/or veterinary purposes.

27. (canceled)

28. A method for the prevention and/or treatment of at least one disease and/or disorder selected from the group consisting of eye diseases; erectile dysfunction; cardiovascular diseases; vascular diseases; inflammatory diseases; proliferative diseases; neurological diseases and disease of the central nervous system; bronchial asthma; osteoporosis; renal diseases; and AIDS comprising administering to an individual in need of such treatment an effective amount of a compound according to claim 1.

29. A method for the prevention and/or treatment of eyes diseases including retinopathy, macular degeneration and glaucoma, and/or for preventing, treating and/or alleviating complications and/or symptoms associated therewith comprising administering to an individual in need of such treatment an effective amount of a compound according to claim 1.

30. A method for the prevention and/or treatment of cardiovascular and vascular diseases selected from the group consisting of acute stroke, congestive heart failure, cardiovascular ischemia, heart disease, cardiac remodeling, angina, coronary vasospasm, cerebral vasospasm, pulmonary vasoconstriction, restenosis, hypertension, pulmonary hypertension, arteriosclerosis, thrombosis including deep thrombosis and platelet related diseases, and/or for preventing, treating and/or alleviating complications and/or symptoms associated therewith comprising administering to an individual in need of such treatment an effective amount of a compound according to claim 1.

31. A method for the prevention, treatment and/or management of neurological and CNS disorders selected from the group consisting of stroke, multiple sclerosis, brain or spinal cord injury, inflammatory and demyelinating diseases comprising Alzheimer's disease, MS and neuropathic pain, and/or for preventing, treating and/or alleviating complications and/or symptoms associated therewith comprising administering to an individual in need of such treatment an effective amount of a compound according to claim 1.

32. A method for the prevention and/or treatment of cancer selected from the group consisting of cancer of the brain (gliomas), breast, colon, intestine, skin, head and neck, kidney, lung, liver, ovarian, pancreatic, prostate, or thyroid; leukemia; lymphoma; sarcoma; melanoma; and/or for preventing, treating and/or alleviating complications and/or symptoms and/or inflammatory responses associated therewith comprising administering to an individual in need of such treatment an effective amount of a compound according to claim 1.

33. A method for the prevention and/or treatment of erectile dysfunction, bronchial asthma, osteoporosis, inflammatory diseases, renal diseases and AIDS, and/or for preventing, treating and/or alleviating complications and/or symptoms associated therewith comprising administering to an individual in need of such treatment an effective amount of a compound according to claim 1.

34. A method for the prevention and/or treatment of inflammatory diseases selected from the group consisting of contact dermatitis, psoriasis, rheumatoid arthritis, inflammatory bowel disease, Crohn's disease and ulcerative colitis, and/or for preventing, treating and/or alleviating complications and/or symptoms and/or inflammatory responses associated therewith comprising administering to an individual in need of such treatment an effective amount of a compound according to claim 1.

35. A method for inhibiting the activity of at least one kinase, in vitro or in vivo using a compound according to claim 1, or a composition comprising such a compound.

36. The method of claim 35 wherein said use is in vitro.

37. The method of claim 35 wherein the at least one kinase is ROCK.

38. The method according to claim 37 in which the at least one kinase is chosen from the alpha and/or beta isoforms of ROCK.

39. The method according to claim 37 in which the at least one kinase is chosen from the alpha isoform of ROCK.


SEQUENCE LISTING (1 sequence)

SEQ ID NO: 1 - protein, 11 aa, Artificial
  S6 Ribosomal Protein-derived peptide
AKRRRLSSLRA